SEQUENCE LISTING (46 sequences)

SEQ ID NO: 1 - protein, 299 aa, Gallus gallus
MDLLGPMEMTEGSLCSFTAADDFYDDPCFNTSDMHFFEDLDPRLVHVGGLLKPEEHPHHHGHHHGNPHEEEHVRAPSGHHQAGRCLLWACKACKRKTTNADRRKAATMRERRRLSKVNEAFETLKRCTSTNPNQRLPKVEILRNAIRYIESLQALLREQEDAYYPVLEHYSGESDASSPRSNCSDGMMEYSGPPCSSRRRNSYDSSYYTESPNDPKHGKSSVVSSLDCLSSIVERISTDNSTCPILPPAEAVAEGSPCSPQEGANLSDSGAQIPSPTNCTPLPQESSSSSSSNPIYQVL

SEQ ID NO: 2 - protein, 297 aa, Anas platyrhynchos
MDLLGAMEMPEGSLCSFTGADDFYDDPCFNTSDMHFFEDLDPRLVHVGGLLKPEEHPHHHGHHHHGHPHEEEHVRAPSGHHQAGRCLLWACKACKRKTTNADRRKAATMRERRRLSKVNEAFETLKRCTSTNPNQRLPKVEILRNAIRYIESLQALLREQEDVYYPVLEHYSGESDASSPRSNCSDGMMEYSGPPCSSRRRNSYDSSYYTESPNDPKHGKSSVVSSLDCLSGIVERISTDNSTCPILPPAETVAEGSPCSPQEGASLNDSGAQIPSPTNCTPLPQESSSSNPIYQVL

SEQ ID NO: 3 - protein, 297 aa, Meleagris gallopavo
MDLLGPMEMTEGSLCSFAAADDFYDDPCFNTSDMHFFEDLDPRLVHVGGLLKPEEHPHHHGHHHGHPHEEEHVRAPSGHHQAGRCLLWACKACKRKTTNADRRKAATMRERRRLSKVNEAFETLKRCTSTNPNQRLPKVEILRNAIRYIGSLQALLREQEDAYYPVLEHYSGESDASSPRSNCSDGMMEYSGPPCSSRRRNSYDSSYYTESPNDPKHGKSSVVSSLDCLSSIVERISTDNSTCPILPPAEAVAEGSPCSPQEGASLNDSGAQIPSPTNCTPLPQDSSSSNNPIYQVL

SEQ ID NO: 4 - protein, 319 aa, Sus scrofa
MELLSPPLRDVDLTGPDGSLCNFATADDFYDDPCFDSPDLRFFEDLDPRLVHVGALLKPEEHSHFPAAAHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYDGTYYSEAPSEPRPGKNAAVSSLDCLSSIVESISTESPAAPALLLADTPRESSPGPQEAAAGSEVERGTPTPSPDAAPQCPASANPNPIYQVL

SEQ ID NO: 5 - protein, 318 aa, Bos taurus
MELLSPPLRDVDLTGPDGSLCNFATADDFYDDPCFDSPDLRFFEDLDPRLVHVGALLKPEEHSHFPAAAHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRSGEHYSGDSDASSPRSNCSDGMMDYSGPPSGARRRNCYDRTYYSEAPNEPRPGKSAAVSSLDCLSSIVERISTESPAAPALLLADAPPESSPGPQEAAGSEVERGTPAPSPDTAPQGLAGANPNPIYQVL

SEQ ID NO: 6 - protein, 300 aa, Oreochromis niloticus
MELSDISFPIPTADDFYDDPCFNTSDMHFFEDLDPRLVHVGLLKPDDSSSSSSSSPSSSSSSPSSLLHLHHHAEVEDDEHVRAPSGHHQAGRCLLWACKACKRKTTNADRRKAATLRERRRLSKVNDAFETLKRCTTANPNQRLPKVEILRNAISYIESLQALLRGGQEDGFYPVLEHYSGDSDASSPRSNCSDGMTDFNGPTCQTTRRGSYDSSSYFSETPNGGLKSERSSVVSSLDCLSSIVERISTDNSSLLPPADGPGSPTTTTTVPVGEAGTAPATAQVSSPTASQDPNLIYQVL

SEQ ID NO: 7 - protein, 227 aa, Gallus gallus
MELFETNPYFFPEQRFYDGENFLGSRLQGYEAAAFPERPEVTLCPESRGALEEKDSTLPEHCPGQCLPWACKICKRKTVSIDRRRAATLREKRRLKKVNEAFEALKRSTLLNPNQRLPKVEILRSAIQYIERLQSLLSSLNQQEREQRELRYRPAAPQPAAPSECGSGSSSCSPEWSTQLEFGTNPADHLLSDDQAEDRNLHSLSSIVESIAVEDVAVTFPEERVQN

SEQ ID NO: 8 - protein, 227 aa, Anas platyrhynchos
MELFETNPYFFPDQRFYDGENFLGSRLQGYEPAAFPEQPEVALCPESRVALEEKDSSLAEHCPGQCLPWACKICKRKTVSIDRRRAATLREKRRLKKVNEAFEALKRSTLLNPNQRLPKVEILRSAIQYIERLQSLLSTLNQQEREQRDLRYRPAGPQPAAASECGSGSSSCSPEWSTQLEFGTNPTDHLLPDEAAEDRNLHSLSSIVESIAVEDVAVAFQEEQVQN

SEQ ID NO: 9 - protein, 227 aa, Meleagris gallopavo
MELFETNPYFFPEQRFYDGENFLGSRLQSYEAAAFPERPEVALCPESRGALEEKDSMLPEHCPGQCLPWACKICKRKTVSIDRRRAATLREKRRLKKVNEAFEALKRSTLLNPNQRLPKVEILRSAIQYIERLQSLLSSLNQQEREQRELRYRPAAPQPAAPSECGSGSSSCSPEWSTQLEFSTNPADHLLSDDAAEDRNLHSLSSIVESIAVEDVAVTFPEERVQN

SEQ ID NO: 10 - protein, 224 aa, Sus scrofa
MELYETSPYFYQEPHFYDGENYLPVHLQGFEPPGYERTELSLSPEARVPLEDKGLGTPEHCPGQCLPWACKVCKRKSVSVDRRRAATLREKRRLKKVNEAFEALKRSTLLNPNQRLPKVEILRSAIQYIECLQALLSSLNQEERDLRYRGGGGPQPGVPSECSSHSASCSPEWGSALEFGPNPGDHLLTADPTDAHNLHSLTSIVDSITVEDVAVAFPDETMPN

SEQ ID NO: 11 - protein, 224 aa, Bos taurus
MELYETSPYFYQEPHFYDGENYLPVHLQGFEPPGYERAELSLSPEARVPLEDKGLGPAEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPAVPSECSSHSASCSPQWGSALEFGPNPGDHLLPADPTDAHNLHSLTSIVDSITVEDVAAAFPDETIPN

SEQ ID NO: 12 - protein, 250 aa, Oreochromis niloticus
MELFETNPYFFPDQRFYEGGDSYFPSRLPGAYDQAGYQDRNSMMGLCGNLSGGVGVGVTGTEDKASPSSMSPHSEPHCPGQCLPWACKLCKRKTVTMDRRRAATLREKRRLKKVNEAFDALKRSTLMNPNQRLPKVEILRSAIQYIERLQALVSSLNQQDTETGQQGLLYRPSPTQPRVSSSSEPSSGSTCCSSPEWSSTPEQCTQSYSSEDLLSAADSPEQGNMRALTSIVDSISAADGPVAFPVDIPK

SEQ ID NO: 13 - protein, 524 aa, Gallus gallus
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAADSSSAYGARHSFSSYSDSFMNAAAPANHMNPVSNGLSPQKQGAQNKMQCSRWNLTIALNNQVMSILSNPSGVPPQPQADFSISPLHGGLDTTNSISASCSQRSDSIKSVDSLPTSQSYCPPTYSTTSYSVDPVAGYQYGQYGQTAVDYLTKNVSLSTQRRMKLGEHSAVLGLLPVETGQAY

SEQ ID NO: 14 - protein, 503 aa, Anas platyrhynchos
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRVATPDVEKKIEEYKRENPGMFSWEIRDRLLKDGHCDRSTVPSGLVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAADTGSAYGARHSFSSYSDSFMNAAAPANHMNPVSNGLSPQVMSILSNPSGVPPQPQADFSISPLHGGLDTTNSISASCSQRSDSIKSVDSLPTSQSYCPPTYSTTSYSVDPVAGYQYGQYGQTAVDYLAKNVSLSTQRRMKLGGHSAVLGLLPVETGQAY

SEQ ID NO: 15 - protein, 520 aa, Meleagris gallopavo
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEEDCDKKEEDGEKKAKHSIDGILGDKGNRLDEGSDVESEPDLPLKRKQRRSRTTFTAEQLEELEKAFERTHYPDIYTREELEKAFERTHYPDIYTREELAQRTKLTEARVQVWFSNRRARWRKQAGANQLAAFNHLLPGGFPPTGMPTLPPYQLPDSTYPTTTISQDGSSTVHRPQPLPPSTMHQGGLAAAAAADSSSAYGARHSFSSYSDSFMNAAAPANHMNPVSNGLSPQVMSILSNPSGVPPQPQADFSISPLHGGLDTTNSISASCSQRSDSIKSVDSLPTSQSYCPPTYSTTSYSVDPVAGYQYGQYGQTAVDYLAKNVSLSTQRRMKLGEHSAVLGLLPVETGQAY

SEQ ID NO: 16 - protein, 503 aa, Sus scrofa
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEDDEADKKEDDSEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPAAPSNHMNPVSNGLSPQVMSILSNPSAVPPQPQADFSISPLHGGLDSATSISASCSQRADSIKPGDSLPTSQSYCPPTYSTTGYSVDPVAGYQYGQYGQTAVDYLAKNVSLSTQRRMKLGEHSAVLGLLPVETGQAY

SEQ ID NO: 17 - protein, 404 aa, Bos taurus
MAALPGTVPRMVRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEEEDEADKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHGFSSYSDSFMNPAAASNHMNPVSNGLSPQWSLTVLFGVQGHHLHLIGAED

SEQ ID NO: 18 - protein, 459 aa, Oreochromis niloticus
MATLPGTVPRMVRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKRIEEYKRENPGMFSWEIRDKLLKDGVCDRSTVPSGNRTDDASDVDSEPDLPLKRKQRRSRTTFTAEQLEELEKAFERTHYPDIYTREELAQRTKLTEARVQVWFSNRRARWRKQAGANQLAAFNHLLPGGFPPTGMPTLPTYQLPESSYPSTTLSQEGSSTLHRPQPLPPSSMHQGGLSADSSSAYGLSSNRHSFSSYSDTFMSPSASSNHMNPVGNGLSPQVMSILSNPSAVPSQPQHDFSISPLHSSLESSNPISASCSQRSDTIKSVDSLASSQSYCPPTYSATSYSVDPVTAGYQYSQYGQTAVDYLAKNVSLSTQRRMKLGDHSAVLGLLQVETGQAY

SEQ ID NO: 19 - protein, 483 aa, Gallus gallus
MTTLAGAVPRMMRPGAGQSYPRGGFPLEVSTPLGQGRVNQLGGVFINGRPLPNHIRHKIVEMAHHGIRPCVISRQLRVSHGCVSKILCRYQETGSIRPGAIGGSKPKVTTPDVEKKIEEYKRENAGMFSWEIRDRLLKDGVCDRNTVPSVSSISRILRSKFGKGEEEEAELERKEAEEGDKKAKHSIDGILSERASAAQSDEGSDIDSEPDLPLKRKQRRSRTTFTAEQLEELERAFERTHYPDIYTREELAQRAKLTEARVQVWFSNRRARWRKQAGANQLMAFNHLIPGGFPPSAMPTLPTYQLSEPSYQPTSIPQAVSDPSSTVHRPQPLPPSTVHQSSLPSNPESSSAYCLPSTRHGFSSYTDSFVPPSGPSNPMNPAIGNGLSPQVMGLLTNHGGVPHQPQTDYALSPLTGGLEPTTTVSASCSQRLDHMKSLDSLPTSQSYCPPTYSTTGYSMDPVTGYQYGQYGQSAFHYLKPDIA

SEQ ID NO: 20 - protein, 482 aa, Anas platyrhynchos
MTTLAGAVPRMMRPGAGQNYPRSGFPLEVSTPLGQGRVNQLGGVFINGRPLPNHIRHKIVEMAHHGIRPCVISRQLRVSHGCVSKILCRYQETGSIRPGAIGGSKPKQVTTPDVEKKIEEYKRENAGMFSWEIRDKLLKDGVCDRNTVPSVSSISRILRSKFGKGEEEEAELERKEAEEGDKKAKHSIDGILSERAPQSDEGSDIDSEPDLPLKRKQRRSRTTFTAEQLEELERAFERTHYPDIYTREELAQRAKLTEARVQVWFSNRRARWRKQAGANQLMAFNHLIPGGFPPSAMPTLPTYQLSEPSYQPTSIPQAVSDPSSTVHRPQPLPPSTVHQSSLPSNPESSTAYCLPSTRHGFSSYTDSFVPPSGPSNPMNPAIGNGLSPQVMGLLTNHGGVPHQPQTDYALSPLTGGLEPTTTVSASCSQRLEHMKSLDSLPTSQSYCPPTYSTTGYSMDPVTGYQYGQYGQSAFHYLKPDIA

SEQ ID NO: 21 - protein, 861 aa, Meleagris gallopavo
MDTICSISTSLGDSSFRLLDSVLQEHYSIALLVCSVCNDSVLVRRPKEKKRARENKEVPRASLLSFQDEEEETEEVFKVKKSSYSKKIVKQLKKEYKEDLEKSKVRTEVNSPTDVEPPLEKTGQIKDIGQEDGTANSEHGEEEMEVESEKEEEKPKPGGAFSSALSSLNVLRPGEIPDAAFIHAARKKRQMARELGDFTPVDSEPGKSRLVREDENDASDDEDDDEKRRIVFTVKEKSQRQKIAEEIGIEGSDDEALVAGEQDEELSRWEQEQIRKGINIPQVQPSQPAEVNNLYYQNTYQTLSYGSSYGIPYTYAAYGSSEAKSQKTDNTVPFKTPSNEMTPITIDLVKKQLKDRLDSMKELHKANRQQFEKHQQSQEDSTKAIERLEGSSGGIGEQYKFLQEMRGYVQDLLECFSEKVPLINELESAMHQLYKQRASRLVQRRQDDIKDESSEFSSHSNKALMAPNLDSFGRDRVLYQEQVKRRTAEREARRARRRQAREQTGKMADHLEGLSSDDEETSTDITNFNMERDRILKESSKVFEDVLESFYSIDCIKSQFEAWRSKYFASYKDAYIGLCLPKLFNPLIRLQLLVWTPLEGKCRDFETMLWFESLLFYGCEEQEQEKDDADISLLPTIVERVVLPKLTVISENIWDPFSTTQTSRMVAIVQKLVNGYPSVVNAENKNTQMLLKALLLRMRRTLDDDVFMPLYPKNVLENKNSGPYLFFQRQFWSSVKLLGNFLQWYGILSNKTLQELSIDGLLNRYILMAFQNSEYGDDSIKKAQSVIACFPKQWFANLKGDKTISQLENFCRYLVHLADTIYRNSIGCSDVEKRNARVANKADHKASSKYTSTGSCCDSCK

SEQ ID NO: 22 - protein, 483 aa, Sus scrofa
MTTLAGAVPRMMRPGPGQNYPRSGFPLEVSTPLGQGRVNQLGGVFINGRPLPNHIRHKIVEMAHHGIRPCVISRQLRVSHGCVSKILCRYQETGSIRPGAIGGSKPKVTTPDVEKKIEEYKRENPGMFSWEIRDKLLKDAVCDRNTVPSVSSISRILRSKFGKGEEEEADLERKEAEESEKKTKHSIDGILSERASAPQSDEGSDIDSEPDLPLKRKQRRSRTTFTAEQLEELERAFERTHYPDIYTREELAQRAKLTEARVQVWFSNRRARWRKQAGANQLMAFNHLIPGGFPPTAMPTLPTYQLSETSYQPTAIPQAVSDPSSTVHRPQPLPPSTVHQSTIPSNPDSSSAYCLPSTRHGFSSYTDSFVPPSGPSNPMNPAIGNGLSPQVMGLLTNHGGVPHQPQTDYALSPLTGGLEPTTTVSASCSQRLEHMKSLDSLPTSQSYCPPTYSTTGYSMDPVTGYQYGQYGQSAFHYLKPDIA

SEQ ID NO: 23 - protein, 484 aa, Bos taurus
MTTLAGAVPRMMRPGPGQNYPRSGFPLEVSTPLGQGRVNQLGGVFINGRPLPNHIRHKIVEMAHHGIRPCVISRQLRVSHGCVSKILCRYQETGSIRPGAIGGSKPKQVTTPDVEKKIEEYKRENPGMFSWEIRDKLLKDAVCDRNTVPSVSSISRILRSKFGKGEEEEADLERKEAEESEKKAKHSIDGILSERASAPQSDEGSDIDSEPDLPLKRKQRRSRTTFTAEQLEELERAFERTHYPDIYTREELAQRAKLTEARVQVWFSNRRARWRKQAGANQLMAFNHLIPGGFPPTAMPTLPTYQLSETSYQPTAIPQAVSDPSSTVHRPQPLPPSTVHQSTLPSNPDSSSAYCLPSTRHGFSSYTDSFVPPSGPSNPMNPAIGNGLSPQVMGLLTNHGGVPHQPQTDYALSPLTGGLEPTTTVSASCSQRLDHMKSLDSLPTSQSYCPPTYSTTGYSMDPVTGYQYGQYGQSAFHYLKPDIA

SEQ ID NO: 24 - protein, 471 aa, Oreochromis niloticus
MTALAGSIPRMMRPSLTQNYPRSGFPLEVSTPLGQGRVNQLGGVFINGRPLPNHIRHKIVEMAHHGIRPCVISRQLRVSHGCVSKILCRYQETGSIRPGAIGGSKPKGTSPDVERRIEEYKRENPVSAISRIMRSKFGGKGDEEEDEDEIEKKELEDNERRAKHSIEGILGDRSSHSDEGSEVESEPDLPLKRKQRRSRTTFTAEQLEELERAFERTHYPDIYTREELAQRAKLTEARVQVWFSNRRARWRKQAGANQLMAFNHLIPGGFPPSAMSSIPPYQLSDSSYPPSSIAQVSEPPSTVHRPQPLPPSSGHQASGGGGQGEGGSAYCLASGRHSFSGYSDSFVSPGAPTNPMNPSIGNGLSPQVMGLLNPGGVPHQPQSDYAISPLTGGLEPPAGMAASCSQRMDHIKGLEGLTSVPSMSALPSLPSSQSYCPPSYSSPGYTVDHVASYQYSQYGQSTLPYLRPEIT

SEQ ID NO: 25 - protein, 398 aa, Gallus gallus
MGRKKIQISRILDQRNRQVTFTKRKFGLMKKAYELSVLCDCEIALIIFNSTNRLFQYASTDMDKVLLKYTEYSEPHESRTNSDILETLKRKGLGLESHELELEEGLDPGEKLRQMNEGMDLTVARPRFYSPVPLPEVPYGSSPSSSSDGALGSASSSPQSQGRPPAFKPTAPKPSGRSPGPMPPGIGYPLFPAGSLNRALATKTPPPLYLGADGQRRSGGSTARPLYSGLQTLNPVLASGSASIPSHSLTGFPFLAPAQAAEFGAGEAPPPPGFLQPGPPAPWQPPRDMAALGASSRIVPTEDLAPGSSPQPHAISIKSERVSPGLGCPSGAPQPSQGSLTSLSEAPQGAADLQPRDDYSKGYPYPPPPPRPLAEEQRATAAVPVPARRAQAEDAWQR

SEQ ID NO: 26 - protein, 406 aa, Anas platyrhynchos
MGRKKIQISRILDQRNRQVTFTKRKFGLMKKAYELSVLCDCEIALIIFNSTNRLFQYASTDMDKVLLKYTEYSEPHESRTNSDILETLKRKGLGLDSHELELDEGLDGGEKMRKLNEGMDLTVARPRFYSPVPLPEASYSSSPPAGGDGALGSTSSSPQSQGRPPAFKPSAPKLSGRSPGPMPPGIGYPLFPPGSLNRALATKTPPPLYLGADGRRGEAHGSLASGRSGGSAARPLYPGLQTLSPVLPPGSAGIPNHSLSGFPFLAPAQAEYGAGEAPPPPGFLQPGPPASWQPPRDMAALGTSTRIVPAEDAAPGSSPQHHAISIKSERVSPGLGCPSGTPQPPPGGLTSLNEAPRGSGDLQPRDDYAKGYPYPLGPPRPLAEEQRATTVPVPPRRAQAVDGWQR

SEQ ID NO: 27 - protein, 397 aa, Meleagris gallopavo
MGRKKIQISRILDQRNRQVTFTKRKFGLMKKAYELSVLCDCEIALIIFNSTNRLFQYASTDMDKVLLKYTEYSEPHESRTNSDILETLKRKGLGLESHELELEEGLDPGEKLRQLNEGMDLTVARPRFYSPVPLPEVPYGSSPPSSSDGALGSASSSPQSQGRPPTFKPTAPKPSGRSPGPMPPGIGYPLFPAGSLNRALATKTPPPLYLGADGQRRSGSGAARPLYSGLQTLSPVLASGSAGIPSHSLTGFPFLTPAQAEFGAGEAPPPPGFLQPGPPAPWQPPRDMAALGASSRIIPTEDLAPGSSPQPHTISIKSERVSPGLGCPSGTPQPSPGSLTSLSEAPRGAADLQPRDDYTKGYPYPLPPPRPLAEEQRATATIPVPARRVQAEDAWQR

SEQ ID NO: 28 - protein, 425 aa, Sus scrofa
MPRPISSRGAGPPALKAPVAAGSSEFCAATAAARCVRERQSECPGGADQGYSTQPGTMGRKKIQISRILDQRNRQVTFTKRKFGLMKKAYELSVLCDCEIALIIFNSANRLFQYASTDMDRVLLKYTEYSEPHESRTNTDILETLKRRGVGLDGPELEPDEGLEGPGEKLRRLAGDGGDPALPRPRLYPAAPTMPSPDMVYGALPPPGCDPSGLGEALPAQSRPSPFRPAAPKAGPPGLAHPLFSPSHLASKTPPPLYLATDGRRPDLPGGLAGARGGLSTSRGLYGSLQSPCSTATPGTPLGSFPFLPAGPPEYGLGDPPPPPGLLQPPTLAPWQPSRGDGPTATPTQSSGGRSLGEEGPPARGASSPTPPVSIKSERLSPAPGGPGDFPKTFPYPLLLARPLAEPLRPGPPMRRLPTADGWPR

SEQ ID NO: 29 - protein, 368 aa, Bos taurus
MGRKKIQISRILDQRNRQVTFTKRKFGLMKKAYELSVLCDCEIALIIFNSSNRLFQYASTDMDRVLLKYTEYSEPHESRTITDILETLKRRGVGLDGPELEPDEGLEGPGEKLRRLAGDGGDPALPRPRLYPAAPTMPSPDMVYGALPPPACEPTGLGEALPAQSRPSPFRPAAPKAGPPGLAHPLFSPSHLASKTPPPLYLAADGRRPDLPGGLAGTRGGLSSSRGLYGGLQSPCSTAAPGPPLGSFPFLPAGPPEYGLGDPPPPPGLLQPPTLAPWQPSRADGPPATPTQPSGGRSLGEDGPPARGASSPTPPVSIKSERLSPAPGGPGDFPKTFPYPLLLARPLAEPLRPGPPLRRLPTADGWPR

SEQ ID NO: 30 - protein, 421 aa, Oreochromis niloticus
MGRKKIQISRILDQRNRQVTFTKRKFGLMKKAYELSVLCDCEIALIIFNSTNRLFQYASTDMDKVLLKYTEYSEPHESRTNTDILETLRRKGLGLDGSELDSEESMQVATDKYPLSEGMDLSVARQRFYGPSLLSPEAQFLVSAGCENGFPNSSGSGMASHRPPSFKSLNSRSSSASPAAPHAHTAFMSPHSGIGYSVFSHGNLNRALDMKSPPPLNVGTENLRADGANQAMGATRANHNSARGLLYQGLHSSSSMVAMGKAGLLSHSLGGYGLPSPGASEYSQPGFYHSVSLQRGTVNPWQAAQPPQEPHGPHINPVESSGGCSFPSQSCTPTSPHLPSLNLSIKSERSSPEHMASPTSPPLHHLRQHSPMSNPDSARHTPPDTHPANGTKEFAKTSYPQDQEEGGQSLRQLEMSDGWQR

SEQ ID NO: 31 - protein, 302 aa, Gallus gallus
MDSFKGGMNLERLPESLRPQPSHDMASSFHLQRSSEPRDPIENSASESSDTEVPEKERSGEQKNEDGAADDPAKKKKQRRQRTHFTSQQLQELEATFQRNRYPDMSMREEIAVWTNLTEPRVRVWFKNRRAKWRKRERNQQMDLCKNGYVPQFSGLMQPYDDMYAGYPYNNWATKSLTPAPLSTKSFTFFNSMSPLSSQSMFSAPSSISSMNMPSGMGHSAVPGMANSGLNNINNISGSSLNSAMSSPACPYGPPGSPYSVYRDTCNSSLASLRLKSKQHSSFGYSSLQSPGSSLNACQYNS

SEQ ID NO: 32 - protein, 406 aa, Anas platyrhynchos; identical to SEQ ID NO: 26

SEQ ID NO: 33 - protein, 397 aa, Meleagris gallopavo; identical to SEQ ID NO: 27

SEQ ID NO: 34 - protein, 314 aa, Sus scrofa
MDAFKGGMSLERLPEGLRPPPPPPHDMGPAFHLARAADPREPLENSASESSDTELPEKERSGEPKGPEDSSAGGAGCGGAEDPAKKKKQRRQRTHFTSQQLQELEATFQRNRYPDMSMREEIAVWTNLTEPRVRVWFKNRRAKWRKRERNQQLDLCKGGYVPQFSGLVQPYEDVYAAGYSYNNWAAKSLAPAPLSTKSFTFFNSMSPLSSQSMFSAPSSISSMTMPSSMGPGAVPGMPNSGLNNINNLTGSSLNSAMSPGACPYGTPASPYSVYRDTCNSSLASLRLKSKQHSSFGYGGLQGPASGLNACQYNS

SEQ ID NO: 35 - protein, 314 aa, Bos taurus
MDAFKGGMSLERLPEGLRPPPPQPHDMGPAFHLARTADPREPLENSASESSDTELPEKERGGEPKGPEDSGAGGAGCGGAEDPAKKKKQRRQRTHFTSQQLQELEATFQRNRYPDMSMREEIAVWTNLTEPRVRVWFKNRRAKWRKRERNQQLDLCKGGYVPQFSGLVQPYDDVYAAGYSYNNWAAKSLAPAPLSTKSFTFFNSMSPLSSQSMFSAPSSISSMTMPSSMGPGAVPGMPNSGLNNINNLTGSSLNSAMSPGACPYGTPASPYSVYRDTCNSSLASLRLKSKQHSSFGYGGLQGPASGLNACQYNS

SEQ ID NO: 36 - protein, 265 aa, Oreochromis niloticus
MELNLSKNVISCGESEKERNAEHRSDDGNADDPKKKKQRRQRTHFTSQQLQELEATFQRNRYPDMSTREEIAVWTNLTEARVRVWFKNRRAKWRKRERNQQMDLCKNSYLPQFSGLMQPYDDMYPAYTYNNWTNKGLTPAPLSTKNFTFFNSMSPLTSQSMFSAPNSISSMTMASGMGHSAVPGMPTPGLNNISNLNGIGTSGINSAMSSSACPYGPPGSPYSVYRDTCNSSLASLRLKSKQHPTFGYSGLQSPGSSLNACQYNS

SEQ ID NO: 37 - protein, 1346 aa, Gallus gallus
MERGAQPGVGVRRLRNVAREEPFAAVLGALRGCYAEATPLEAFVRRLQEGGTGEVEVLRGDDAQCYRTFVSQCVVCVPRGARAIPRPICFQQLSSQSEVITRIVQRLCEKKKKNILAYGYSLLDENSCHFRVLPSSCIYSYLSNTVTETIRISGLWEILLSRIGDDVMMYLLEHCALFMLVPPSNCYQVCGQPIYELISRNVGPSPGFVRRRYSRFKHNSLLDYVRKRLVFHRHYLSKSQWWKCRPRRRGRVSSRRKRRSHRIQSLRSGYQPSAKVNFQAGRQISTVTARLEKQSCSSLCLPARAPSLKRKRDGEQVEITAKRVKVMEKEIEEQACSIVPDVNQSSSQRHGTSWHVAPRAVGLIKEHYISERSNSEMSGPSVVRRSHPGKRPVADKSSFPQGVQGNKRIKTGAEKRAESNRRGIEMYINPIHKPNRRGIERRINPTHKPELNSVQTEPMEGASSGDRKQENPPAHLAKQLPNTLSRSTVYFEKKFLLYSRSYQEYFPKSFILSRLQGCQAGGRRLIETIFLSQNPLKEQQNQSLPQQKWRKKRLPKRYWQMREIFQKLVKNHEKCPYLVFLRKNCPVLLSEACLKKTELTLQAALPGEAKVHKHTEHGKESTEGTAPNSFLAPPSVLACGQPERGEQHPAEGSDPLLRELLRQHSSHWQVYGFVRECLERVIPAELWGSSHNKCRFFKNVKAFISMGKYAKLSLQQLMWKMRVNDCVWLRLAKGNHSVPAYEHCYREEILAKFLYWLMDSYVIELLKSFFYITETMFQKNMLFYYRKFIWGKLQNIGIRDHFAKVHLRALSSEEMEVIRQKKYFPIASRLRFIPKMNGLRPVVRLSRVVEGQKLSKESREKKIQRYNTQLKNLFSVLNYERTVNTSIIGSSVFGRDDIYRKWKEFVTKVFESGGEMPHFYFVKGDVSRAFDTIPHKKLVEVISQVLKPESQTVYGIRWYAVIMITPTGKARKLYKRHVSTFEDFIPDMKQFVSKLQERTSLRNAIVVEQCLTFNENSSTLFTFFLQMLHNNILEIGHRYYIQCSGIPQGSILSTLLCSLCYGDMENKLLCGIQKDGVLIRLIDDFLLVTPHLMQARTFLRTIAAGIPEYGFLINAKKTVVNFPVDDIPGCSKFKHLPDCRLISWCGLLLDVQTLEVYCDYSSYAFTSIRSSLSFNSSRIAGKNMKCKLTAVLKLKCHPLLLDLKINSLQTVLINIYKIFLLQAYRFHACVLQLPFNQKVRNNPDFFLRIISDTASCCYFILKAKNPGVSLGSKDASGMFPFEAAEWLCYHAFIVKLSNHKVIYKCLLKPLKVYKMHLFGKIPRDTMELLKTVTEPSLCQDFKTILD

SEQ ID NO: 38 - protein, 1349 aa, Anas platyrhynchos
MEEGRREGEGRGAAAAPGAAAAAAGDGGAGGTGSTGGTGGGRPHRNMAGAEPFGAVLGALRGCYAQVAPLETFVRGLGESGAEEAEVVRDSDAASYRTFVSQCVVCVPHGARDIPRPFSLEQLSSQSEVISRVMQRLCGKKKKNILTYGYSLLDENSSHFQIMPLSNVYSYLPNTATETMRISGLWETLLSRIGDDVMMYLLEHCAIFMLVPPSNCYQVCGQPIYELISQNVESAPEFVKQRLSKHKRSSLLKYMQKRLTFHRQYLSKSRQSKRRQRLEANVSSVRNKTSNNIQSLGSAALEKQSSSNAGLSATAPSLKRKLAREQLEVTAKRARLEEKEREEQACNTAPNVNQSIPKRYGTGCVASRSVSLTKEKNISQRSNSDMSRPSLVHNSHRGKKSVADKSSFLQGAESNRHLKPSIEMQAGSSRKGVETRRPIPRLDWVPIEPAESSSSGHKKQEGPLAHLAEEVPNRVLPSTIYIDRKFLYSRRYWGERFPKSFLLNRLKGSQAGVKRLIETIFLSQNPFGQKCNQGLPQKKRRKKKLPKRFWRMRSIFQQLLKNHGKFPYVAFLRQNCPLRISDTILGKAKLLSRAPLPGQAEARKQAEQLGKEPAERVASSRCESGHTNVPSSVRAPLAASACGEPGGEEQIPAEASDSVLRELLKEHCSHFQVYLFVRECVERVIPTELWGSNHNKRRFFKNVKAFISMGKYAKLSLQVLMWKMRVNDCMWLRLAKGNHFVPASEHLYREEILAKFLYWLMDTYVVQLLRSFFYVTETMFQKNMLFYYRKCIWGKLQDIGIRKHFSKVKLRPLTAEEMEAIHQKKYLPMASKLRFIPKVTGLRPIVRMSGVVEAQTLSKESRAKKMNHYNMQLKNLFSVLNYERTINTSYIGSSVFGRDDIYKSWKTFVKKVLKSDGEIPHFYYVKADVSRAFDSIPHNKLVEVISQVLKPEKKTVYCIRRYAVVMITGSGKTRKLYKRHVSTFKDFMPDMKQFVSRLHESTSLRDAIIVEQSLTFNETSASLFNFFLQMLNNNILEIERSYYLQCSGIPQGSLLSTLLCSLCYGDMENKLFSGVQKDGVLIRLIDDFLLVTPHLTHARTFLRTLAMGIPEYGFLINPKKTVVNFSADDIPECSEFKQLPNCRLIPWCGLLLDTQTLEVYCDYSSYSCTSIRSSLSFNSNRTAGKNMKHKLLAVLKLKCHGLFLDLQINSLKTVFINVYKIFLLQAYRFHACVIQLPFNQKVRNNPDFFLRVIAENASCCYSMLKAKNPGFTLGNRGASGMFPSEAAEWLCYHAFTVKLSNHKVVYKCLLKPLKFCMMQLFRKIPKDTKALLKTVTEPSICKDFKSILD

SEQ ID NO: 39 - protein, 1352 aa, Meleagris gallopavo
MEASRNIEFSGGAACASAFWGTVTWRPGVGSRGGEERHRVLPGHEEQDQKGSTEQASVRRNVRLKSRPGCLAAVSASLGHVLFVEKLLIKKSVLPFSVFVQLSSQSEVITRIVQRLCEKKKKNILAYGYSLLDENSCHFRILPSSCIYSYLPNTVTETIRISGLWEILLSRIGDDVMMYLLEHCALFMLVPPSNCYQVCGQPIYELISRNIGPSPGFVRRRYSRFKHNNLLNYVRKRLVFHRHYLSKSQWWKCGPRRQGRVSSRRKRRTHRIQSPRSGYQSSAKVNFQAGMRISTVTAHLEKQNCSSLCLPARTPSLKRKRDGEQVETTAKRVKVMEREEQACSIVPDVNRSSSRRHGVWHVAPRAVGLIKERYVSERSYSEMSGPSVVHRSHPGKRPVADKSSFPRGVQGNKHIKTGAEKRAESNKRGIEMYINPICKPNRRGIERHINPTHKPGLNSVQTEPMESASSGDRKQENPPAHLAKQLPNTFLRSAVYFEKKFLLYSRSYQEYFPKSFILSRLQGCQAGGRQLIETIFLSQNPLKEKQNQSLKQQKWRKKRLPKRYWQMREIFQKLLKNHEKCPYLVFLRKNCPVLLSEACLKKTELTLQAALPGEAKVHKHTEHGEETTEGTAPNSFYTPPSMPLCGQTEREEQHLAEGSDPLLRELLRQHSSHWQVYGFVRECLERVIPAELWGSSHNKCRFFKNVKAFISMGKYAKLSLQQLMWKMRVNDCVWLRLAKGNHSVPAYEHCYREEILAKFLYWLMDSYVIELLKSFFYITETMFQKNMLFYYRKFIWGKLQNIGIRNHFAKVHLRALSSEEMEVIHQKKYFPIASRLRFIPKINGLRPVVRLSRVVEGQKLSKESREKKIQRYNTQLKNLFSVLNYERTVNTSIIGSSVFGRDDIYRKWKEFVTKVFESGGEMPHFYFVKGDVSRAFDTIPHKKLVEVISQVLKPESQTVYGIRWYAVIMITPTGKARKLYKRHVSTFEDFIPDMKQFVSKLQERTSLRNAIVVEQCLTFNENSSTLFTFFLQMLHNNILEIGHRYYIQCSGIPQGSILSTLLCSLCYGDMENKLLCGIQKDGILIRLIDDFLLVTPHLMQAKTFLRTIAAGIPEYGFLINAKKTVVNFPVDDIPGCSKFKQLPDCRLISWCGLLLDMQTLEVYCDYSSYAFTSIRSSLSFNSSRIAGKNMKCKLTAVLKLKCHPLFLDLKINSLKTVLINIYKIFLLQAYRFHACVLQLPFNQKVRNNPYFFVRIISDTASCCYFILKAKNPGVCLGCKDASGMFPFEAAEWLCYHAFIVKLSNHKVIYKCLLKPLKVYKMHLFGKIPRDTMVLLKTVTEPSLCQDFKTILD

SEQ ID NO: 40 - protein, 1131 aa, Sus scrofa
MPRAPRCRAVRSLLRDRYRQVLPLATFVRRLGPEGRRLVRRGDPAAYRALVAQCLVCVPWDAQPPPASPSFRQVSCLKELVARVVQRLCERGARNVLAFGFALLDGARGGPPVAFTTSVRSYLPNTVTDTLRGSGAWGLLLRRVGDDVLTHLLARCALYLLVPPSCAYQVCGPPLYDLYTAAEARPMRHKGQTPTGLGLTRPVCNGEAGRPQEQRAQGVRRRRGRAGGHPLPAKRPRHVPEPEQGPEGQASRAHQGRAPGPSDSDPPVMTPTRAAAKAKSREGEAPGTRHLSPQAGGARGTCPPSWWQPHLQGKPSPHVCAAETKRFLYCSGSKEGLRRSFLLCSLPPSLAGAGRLVEVIFLASKPGQPGARRVPARYWRMRPLFRELLKNHARCPYKALLRAHCPLRAAATLSGSGGQVCDHKVGPLAPERLAAAAEGDSASRRLVQLLRQHSSPWQVYRLLRACLHRLVPPGLWGSPHNKRRFLKNVKKLVSLGKHARLSLQELMWKMKVQDCIWLRRSPDARHVQAAEHRLREAILAKFLRWLMGTYVVELLRSFFYVTETTFQKNRLFFFRKRIWSRLQSAGIRQHLDRVRLRELSEAEIRRRREARPAVLTSKLRFVPKPDGLRPIVNMANVVRARTGPGDKKVRRLTGQVKTLFAVLNYERARRPRLLGASVLGVGDIHRAWRAFVLPLRAQDPAPPLYFVKVDVTGAYDALPQDRLLEVVANVIRPHESTYCVRQCAVLRRTARGHVRKSFQTHVSTFADLQPYMRQFVAHLQATGPLRDAVVIEQSCSLNEAGSRLLELFLSLLRNHVIRIGGRSYVQCQGIPQGSILSTLLCSLCYGDMENRLFPGIQRDGVLLRLVDDFLLVTPHLTRAKAFLRTLVRGVPEYGCLANLRKTAVNFPVEDGARGGPAPLQLPAHCLFPWCGLLLDTRTLEVHCDYASYARTSIRASLTFNQGFKPGRNMRRKLLAVLRLKCHGILLDLQVNSLPTVLANVYKIFLLQAYRFHACVLQLPFRQPLARNPSFFLRLVSDTASCCYSLLKARNAGMSLGARGASGPFPSEAAEWLCLHAFLLKLVRHRVTYSCLLGPLRAARERLCQRLPGATLAALEAAADPALTTDFRTILD

SEQ ID NO: 41 - protein, 1125 aa, Bos taurus
MPRAPRCRAVRALLRASYRQVLPLAAFVRRLRPQGHRLVRRGDPAAFRALVAQCLVCVPWDAQPPPAAPSFRQVSCLKELVARVVQRLCERGARNVLAFGFTLLAGARGGPPVAFTTSVRSYLPNTVTDTLRGSGAWGLLLHRVGDDVLTHLLSRCALYLLVPPTCAYQVCGPPLYDLRAAAAAARRPTRQVGGTRAGFGLPRPASSNGGHGEAEGLLEARAQGARRRRSSARGRLPPAKRPRRGLEPGRDLEGQVARSPPRVVTPTRDAAEAKSRKGDVPGPCRLFPGGERGVGSASWRLSPSEGEPGAGACAETKRFLYCSGGGEQLRRSFLLCSLPPSLAGARTLVETIFLDSKPGPPGAPRRPRRLPARYWQMRPLFRKLLGNHARSPYGALLRAHCPLPASAPRAGPDHQKCPGVGGCPSERPAAAPEGEANSGRLVQLLRQHSSPWQVYGLLRACLRRLVPAGLWGSRHNERRFLRNVKKLLSLGKHGRLSQQELTWKMKVQDCAWLRASPGARCVPAAEHRQREAVLGRFLHWLMGAYVVELLRSFFYVTETTFQKNRLFFFRKRIWSQLQRLGVRQHLDRVRLRELSEAEVRQHQEARPALLTSRLRFVPKPGGLRPIVNVGCVEGAPAPPRDKKVQHLSSRVKTLFAVLNYERARRPGLLGASVLGMDDIHRAWRAFVLPLRARGPAPPLYFVKVDVVGAYDALPQDKLAEVIANVLQPQENTYCVRHCAMVRTARGRMRKSFKRHVSTFSDFQPYLRQLVEHLQAMGSLRDAVVIEQSCSLNEPGSSLFNLFLHLVRSHVIRIGGRSYIQCQGIPQGSILSTLLCSFCYGDMENKLFPGVQQDGVLLRLVDDFLLVTPHLTRARDFLRTLVRGVPEYGCQVNLRKTVVNFPVEPGALGGAAPLQLPAHCLFPWCGLLLDTRTLEVHGDHSSYARTSIRASLTFTQGFKPGRNMRRKLLAVLQLKCHGLFLDLQVNSLQTVFTNVYKIFLLQAYRFHACVLQLPFSQPVRSSPAFFLQVIADTASRGYALLKARNAGASLGARGAAGLFPSEAAQWLCLHAFLLKLARHRVTYSRLLGALRTARARLHRQLPGPTRAALEAAADPALTADFKTILD

SEQ ID NO: 42 - protein, 1022 aa, Oreochromis niloticus
MKICSLPELLAFVLNTLKRKRKRNVLAHGYNFQTLAQEDRDADFLKFQGDVTQSAAYIHGSDLWKKVTMRLGTDITQYLLESCSVFVAVPPSCVFQVCGPPVYDRVSMTMASSGFFLQPGVRKHNRTKIESCRGSVSLKQKRTVVNPAASKKMKRRNKGGKKGKRKRETGEEEEVAVCSRKRRRVASIEHQQAIQPVGSEKEGQVVPVESAPPAAFKQPVEMPTLEGGPSWRSGIFPPLPPSQCFIRTLGFLYGGRGMRGFLLNRRKKTAHGSRRLQGQDLVRIVFFEGLAYLNGVERKPKKLPQRFFGMVPLFRQLLQQHRSCSYTKILQRLCPSIEESNAGQGELNSLLPQHCAPHRVYLFVRECLSSVIPQELWGSDQNRLHFFARVRTFLRSGKFERLSLAELMWKIKVNDCDWLKRSKTGCFPPSELAYRTQVLGQFLAWLLDGYVTGLVRACFYATESIGQKNAIRFYRQEVWAKLQDLAFRGHLSKGQMEELTPAQVASLPKGTVISRLRFIPKTDGMRPITRVIGADAKTRLYRGRVRDLLDMLRACVRATPSLLGSTVWGMTDIHKVLCSLAPAQKEKPQPLYFVKVDVSGAYESLPHDKLIEVIGQALSPVHDELFTIRRYAKIWADSHEGLKKAFVRQADFLEDNMGSTNMKGFLTSLQRKGKVHHAILVEQHFCSDLHGREALQFFTQMLTGSVVQYGKKTYRQCRGIPQGSVVSSLLCCLCYGHMENLLFKDIPGHKGCLMRLVDDFLLITPDQHEAQAFLKILLAGVPQYGLAVNPQKVVLNFQVSGSVASCPDIRILPPHCLFPWCGLLLDTHKLDVYKDYSSYAGLSLRYSLTLGSSHSAGQQMKRKLMAILRLKCHALFFDLKTNSLEAVYKNIYKLVLLHACRFHVCAQSLPFGQTVSKNPVFFLQLIWEMAQYCNKLIRRSNKGLILGDKAQTGIVQYEAVELLFCLCFLLVLSQHRLLYKDLLAHLHKRKRSLERRLGDLRLARVRQAASPRTPVDFLAIQT

SEQ ID NO: 43 - DNA, 927 bp, Synthetic construct
  source 1..927: mol_type = other DNA
atggacttactgggccccatggaaatgacggagggctccctctgctccttcacggccgccgatgacttctatgacgacccgtgcttcaacacgtcggacatgcacttcttcgaggacctggacccccggctggtgcacgtgggcgggctgctgaaagccgaggagcacccgcaccaccacgggcaccaccacgggaacccacacgaggaggagcacgtgcgggcgcccagtgggcaccaccaggccggccgctgcctgctgtgggcgtgcaaggcctgcaagaggaagaccaccaacgctgaccgccgcaaagccgccaccatgagggaacggcggcggctcagcaaggtcaacgaggcctttgagaccctcaagcgctgcacttccaccaaccccaaccagcgcctgcccaaggtggagatcctgcgcaacgccatccgctacatcgagagcctgcaggccctgctgcgtgagcaggaggatgcatactacccagtgctggagcactacagcggggagtcagatgcctccagccctcgctccaactgctccgacggcatgatggagtacagcgggccgccctgtagctctcgcaggagaaacagctacgacagcagctactacacggaatcaccaaatgacccaaagcatgggaagagttctgttgtttccagcctcgactgcctctcaagcattgtggagaggatttccacagacaactccacatgtcccatactgcctccagctgaagctgtagctgaagggagtccctgttccccccaggaaggaggaaacctgagtgacagtggagcccagattccttcccccaccaactgcacccctcttccccaggaaagcagcagcagcagcagcagcaatccaatctaccaagtgctataaaaaaaaaaaaaaaaaaaaaaaaaaaaa

SEQ ID NO: 44 - DNA, 6725 bp, synthetic construct
  source 1..6725: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgcgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacgcggccgccagatcttccggatggctcgagtttttcagcaagatatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatagggccgggattctcctccacgtcaccgcatgttagaagacttcctctgccctcgcgagatccggtggagccgggtccggcggtgccgtccacggcagaattggacgactgagcgcgggatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgcttgggggtgccctctccgccgcccaccagctcgaactccacgccgttcagggtgccggtgatgcggcactcgatctccatggcgggcaggccgctctcgtcgctctccatggtggcgtctagcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttatctttctagaaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagcagctgggaagggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctggatccgccaccatggacttactgggccccatggaaatgacggagggctccctctgctccttcacggccgccgatgacttctatgacgacccgtgcttcaacacgtcggacatgcacttcttcgaggacctggacccccggctggtgcacgtgggcgggctgctgaaagccgaggagcacccgcaccaccacgggcaccaccacgggaacccacacgaggaggagcacgtgcgggcgcccagtgggcaccaccaggccggccgctgcctgctgtgggcgtgcaaggcctgcaagaggaagaccaccaacgctgaccgccgcaaagccgccaccatgagggaacggcggcggctcagcaaggtcaacgaggcctttgagaccctcaagcgctgcacttccaccaaccccaaccagcgcctgcccaaggtggagatcctgcgcaacgccatccgctacatcgagagcctgcaggccctgctgcgtgagcaggaggatgcatactacccagtgctggagcactacagcggggagtcagatgcctccagccctcgctccaactgctccgacggcatgatggagtacagcgggccgccctgtagctctcgcaggagaaacagctacgacagcagctactacacggaatcaccaaatgacccaaagcatgggaagagttctgttgtttccagcctcgactgcctctcaagcattgtggagaggatttccacagacaactccacatgtcccatactgcctccagctgaagctgtagctgaagggagtccctgttccccccaggaaggaggaaacctgagtgacagtggagcccagattccttcccccaccaactgcacccctcttccccaggaaagcagcagcagcagcagcagcaatccaatctaccaagtgctataagctagcatcgatcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttgtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 45 - DNA, 10049 bp, synthetic construct
  source 1..10049: mol_type = other DNA
tggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaagcttgcatgcctgcaggtcgacctgcagcgatcgaacttgtttattgcagcttataatggttacaaataaagcaatagcatcacaaatttcacaaataaagcatttttttcactgcattctagttgtggtttgtccaaactcatcaatgtatcttatcatgtctggatcccttctgagggcagaggaagtcttaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcccatggtggcggcacgtgaggtaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaattggtggcgatgccgagctcggtaccaagcttaagtttaaacgctagcagtagaattcggatcctaccgttcgtatagcatacattatacgaagttatgtcgacgatgtaggtcacggtctcgaagccgcggtgcgggtgccagggcgtgcccttgggctccccgggcgcgtactccacctcacccatctggtccatcatgatgaacggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacggcgcgccgatatcgttaactctagagccatgagtacaccctgctggaatcacgagggctttcactgctgctgcctcgtggctgctcgccgccccgggttcagaagcgaaaggctggggctgccagcaccttcggtggctttcgtcagcacccgtcagccgggaacatgtggcgtgggggcactgcgcgcagtttggagctctcaagcagaagccacgaatccttgccgctgaagcgattctctctggccgcatctgaaacgccaggccagaggaggtctgtgcaagtaactactcccggagctcgctgaccgcctcccctcgggccgcagccgcgggtcagaggggcagagctcggggactctacggctgccgtgctgcgcgggaggcggcggagcggcaacgccgtcccgccctgcagtgctccgggcgctgccgtccggcctgggggcggcgagcccctcccggcagtcgctcgtgactcccgcccgatccggccgccgcggggctctgctcctcagccccgcccctccccgccgctcctcagcggagcgccgcgtgccgcgctgccgggcggggcgttgcgcgggggccgccgggagcggtgacgcgtcgccgaggccccgccccgtcgcagagtatataaggggcggcgctcacgccgtcgttcttttccgctccgggtttgccgacgccgtgcgggtgaggcgcgtgcggcggggcaggccgggcccgggggtggggggtgcggggtgcggggatgctcggggggagcctcgttgcaccgtgagcgcggggccggggcgcggccgcggagaggcgcgtgggaacggcggagtggggagggggaagcgagatgagggacgggtgggagagagcggggcgagcgccggggtaaagccgacgtgtcccgcctgaggacccacgcggggcagcgcggcgatgagcacgcggggccccaccgccgccttggcttcgcgcggagccgcccggcgctggggcgcggcgtacggtgctcggcctcgggcgggtggcggccggggaccccggggccgcgccgtcggtgccgcggtgggggaggggaggcgaggccggagcacgagaaaaaaaatggccgcccgctcttcctgccgccgccagctcacaaaatggaggacggcggcccgggcggggtgagtcacacacaaaggagaggggcggccccgccctccgcgccgcccgtggccgcgccgccgccccgcgcccccaccccagcccctgcccggcccggccgccctgcggggagggggcgacggcggaggcgcgctgtaggggaccccgggccaccgcccgcccctccgctcccctcccccctcctcccccccctggcggggcggggccggggctgcacgggccctctgccccctcggccggcgcccggcgggcggcgctgctgcgggacgggggcggccgcagtaaccgggattcgttctggaactttcaggtgtcgtttctctttggccggaagaaagaagctaaagaccatccgaatcgcgacgccaccatggcagcgctccccgggacggtaccgcggatgatgcgcccggcgccggggcagaactacccgcgcaccggcttccctttggaagtgtccaccccgctgggccagggccgggtgaaccaactcggaggggttttcatcaacgggcgcccactgcccaaccacatccgccataagatcgtggagatggctcaccacggcatccggccctgcgtgatctccaggcagctgagggtctcccacggctgcgtttccaaaatcctctgcaggtaccaagagacgggctccatccggcccggggccatcgggggcagcaagcccaggcaggttgcgactcccgacgtggagaagaaaatcgaggaatacaagagggagaaccctgggatgttcagctgggagatccgggacaggctgctgaaggacggacactgcgaccgcagcactgtgccctcagtgagttcgattagccgtgtgctacgcatcaaattcgggaagaaagaggaagaggaggactgcgacaagaaggaggaagacggggagaagaaggccaagcacagcatagatggcatcctgggcgacaaagggaacaggctggatgaaggctccgatgtcgaatcagaaccagacctgcctttgaagaggaagcagcgccgcagccggaccactttcactgccgagcagctggaggagctggagaaggcctttgagaggacccactacccggacatctacaccagggaggagctggcacagagaaccaagctcaccgaggcccgtgttcaggtgtggttcagcaaccgacgagcaagatggcgcaagcaggcgggtgcaaaccaactcgcagcattcaaccatctgctgccagggggattcccacccacgggaatgccaactctgcccccgtaccagctgccagactccacctacccaaccaccaccatttcccaagatggaggcagcaccgtgcacagaccccagcccttgccaccatccaccatgcaccagggagggctcgctgccgccgctgcagccgactccagctctgcctatggggcccgacacagcttctccagctactcagacagcttcatgaatgctgcagctcctgccaaccacatgaatcctgttagcaatggcctctctccgcagaagcagggtgcccaaaacaagatgcagtgctccaggtggaacctcaccatagccttgaacaatcaggtgatgagcatcctgagcaaccccagcggggttcctccgcagccccaggctgacttctccatctctcctcttcacggtggcctggacaccaccaactccatctctgccagctgcagccagcggagtgactccatcaagtccgtggacagcctcccgacctcgcagtcctactgtcctcccacctacagcaccaccagttacagcgtggacccggtggctggctaccagtatgggcagtatggacaaactgctgttgattatttgaccaagaacgtgagcctgtccacgcagcgcaggatgaagctgggagaacattcggccgttctggggctcctaccagtagagacaggccaagcttacgccacaaacttctctctgctaaagcaagcaggtgatgttgaagaaaaccccgggcctggccggaaaaaaatccagatcagccgaatattggatcagcggaaccggcaggtgaccttcaccaagcggaaattcgggctgatgaagaaggcgtatgagctgagcgtgctgtgcgactgcgagatcgccctcatcatcttcaacagcaccaaccgcctcttccagtacgccagcaccgacatggacaaagtgctgctcaagtacacggagtacagcgaaccccacgagagccgcaccaactccgacatcctcgagacgctgaagcgcaaagggttggggctggagagccacgagctggagctggaagaggggctggatcctggtgagaagctgcggcagatgaatgagggcatggatctgacggtggcacggccccgcttttacagcccggtgccgctgcccgaggtcccctatggcagctccccgtcgagcagcagtgatggagccctgggcagcgccagcagctccccgcagagccagggccgcccgcccgccttcaaacccacagcgccgaagccatcgggacgctcaccaggaccgatgcccccaggtattggctaccccctgttccccgctggcagcctgaaccgagccttggcaaccaagaccccaccaccgctgtacctgggagccgatgggcagcgccggagcggaggcagcactgcacggccgctgtactctggtctgcagaccttgaacccagtgctcgcctcaggcagcgccagcattcccagccacagcctcactggtttccccttcctcgctccggcacaagcagcagagttcggggctggcgaggccccaccacctcctggcttcctgcagcctggccctccagctccatggcagcccccacgggacatggcagcactaggggccagcagcaggatcgtccccaccgaggacctggcccccggcagctccccgcagccccacgccatcagcatcaagtcagagcgggtgtccccagggctcggctgcccctcaggtgccccacagccctctcagggcagcctgacctccctgagcgaagccccccaaggcgctgcagacctccagccacgggatgactactccaagggctacccctaccccccgcccccgccccggccgctggcggaggagcagagggccactgccgccgtcccggtccccgcgcggcgggcgcaggctgaggatgcgtggcagagagagggcaggggaagtcttctaacatgcggggacgtggaggaaaatcccggcccagacttactgggccccatggaaatgacggagggctccctctgctccttcacggccgccgatgacttctatgacgacccgtgcttcaacacgtcggacatgcacttcttcgaggacctggacccccggctggtgcacgtgggcgggctgctgaaagccgaggagcacccgcaccaccacgggcaccaccacgggaacccacacgaggaggagcacgtgcgggcgcccagtgggcaccaccaggccggccgctgcctgctgtgggcgtgcaaggcctgcaagaggaagaccaccaacgctgaccgccgcaaagccgccaccatgagggaacggcggcggctcagcaaggtcaacgaggcctttgagaccctcaagcgctgcacttccaccaaccccaaccagcgcctgcccaaggtggagatcctgcgcaacgccatccgctacatcgagagcctgcaggccctgctgcgtgagcaggaggatgcatactacccagtgctggagcactacagcggggagtcagatgcctccagccctcgctccaactgctccgacggcatgatggagtacagcgggccgccctgtagctctcgcaggagaaacagctacgacagcagctactacacggaatcaccaaatgacccaaagcatgggaagagttctgttgtttccagcctcgactgcctctcaagcattgtggagaggatttccacagacaactccacatgtcccatactgcctccagctgaagctgtagctgaagggagtccctgttccccccaggaaggaggaaacctgagtgacagtggagcccagattccttcccccaccaactgcacccctcttccccaggaaagcagcagcagcagcagcagcaatccaatctaccaagtgctataatcgaggttaacgcggccgccacgtgctcgaggagttgccagccatctgttgtttgcccctcccccgtgccttccttgaccctggaaggtgccactcccactgtcctttcctaataaaatgaggaaattgcatcataacttcgtatagcatacattatacgaacggtaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagc

SEQ ID NO: 46 - DNA, 6956 bp, synthetic construct
  source 1..6956: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgacctgcagcgatcgaacttgtttattgcagcttataatggttacaaataaagcaatagcatcacaaatttcacaaataaagcatttttttcactgcattctagttgtggtttgtccaaactcatcaatgtatcttatcatgtctggatcccttctgagggcagaggaagtcttaatggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatagccatggtggcgacgaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccacccttcccagctgctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaattggtggcgatgccgagctcggtaccaagcttaagtttaaacgctagcagtagaattcggatcctaccgttcgtatagcatacattatacgaagttatgtcgacgatgtaggtcacggtctcgaagccgcggtgcgggtgccagggcgtgcccttgggctccccgggcgcgtactccacctcacccatctggtccatcatgatgaacggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacggcgcgccgatatcgccatgagtacaccctgctggaatcacgagggctttcactgctgctgcctcgtggctgctcgccgccccgggttcagaagcgaaaggctggggctgccagcaccttcggtggctttcgtcagcacccgtcagccgggaacatgtggcgtgggggcactgcgcgcagtttggagctctcaagcagaagccacgaatccttgccgctgaagcgattctctctggccgcatctgaaacgccaggccagaggaggtctgtgcaagtaactactcccggagctcgctgaccgcctcccctcgggccgcagccgcgggtcagaggggcagagctcggggactctacggctgccgtgctgcgcgggaggcggcggagcggcaacgccgtcccgccctgcagtgctccgggcgctgccgtccggcctgggggcggcgagcccctcccggcagtcgctcgtgactcccgcccgatccggccgccgcggggctctgctcctcagccccgcccctccccgccgctcctcagcggagcgccgcgtgccgcgctgccgggcggggcgttgcgcgggggccgccgggagcggtgacgcgtcgccgaggccccgccccgtcgcagagtatataaggggcggcgctcacgccgtcgttcttttccgctccgggtttgccgacgccgtgcgggtgaggcgcgtgcggcggggcaggccgggcccgggggtggggggtgcggggtgcggggatgctcggggggagcctcgttgcaccgtgagcgcggggccggggcgcggccgcggagaggcgcgtgggaacggcggagtggggagggggaagcgagatgagggacgggtgggagagagcggggcgagcgccggggtaaagccgacgtgtcccgcctgaggacccacgcggggcagcgcggcgatgagcacgcggggccccaccgccgccttggcttcgcgcggagccgcccggcgctggggcgcggcgtacggtgctcggcctcgggcgggtggcggccggggaccccggggccgcgccgtcggtgccgcggtgggggaggggaggcgaggccggagcacgagaaaaaaaatggccgcccgctcttcctgccgccgccagctcacaaaatggaggacggcggcccgggcggggtgagtcacacacaaaggagaggggcggccccgccctccgcgccgcccgtggccgcgccgccgccccgcgcccccaccccagcccctgcccggcccggccgccctgcggggagggggcgacggcggaggcgcgctgtaggggaccccgggccaccgcccgcccctccgctcccctcccccctcctcccccccctggcggggcggggccggggctgcacgggccctctgccccctcggccggcgcccggcgggcggcgctgctgcgggacgggggcggccgcagtaaccgggattcgttctggaactttcaggtgtcgtttctctttggccggaagaaagaagctaaagaccatccgaatcgcgacgccaccatgtacccatacgatgttccagattacgctgacttactgggccccatggaaatgacggagggctccctctgctccttcacggccgccgatgacttctatgacgacccgtgcttcaacacgtcggacatgcacttcttcgaggacctggacccccggctggtgcacgtgggcgggctgctgaaagccgaggagcacccgcaccaccacgggcaccaccacgggaacccacacgaggaggagcacgtgcgggcgcccagtgggcaccaccaggccggccgctgcctgctgtgggcgtgcaaggcctgcaagaggaagaccaccaacgctgaccgccgcaaagccgccaccatgagggaacggcggcggctcagcaaggtcaacgaggcctttgagaccctcaagcgctgcacttccaccaaccccaaccagcgcctgcccaaggtggagatcctgcgcaacgccatccgctacatcgagagcctgcaggccctgctgcgtgagcaggaggatgcatactacccagtgctggagcactacagcggggagtcagatgcctccagccctcgctccaactgctccgacggcatgatggagtacagcgggccgccctgtagctctcgcaggagaaacagctacgacagcagctactacacggaatcaccaaatgacccaaagcatgggaagagttctgttgtttccagcctcgactgcctctcaagcattgtggagaggatttccacagacaactccacatgtcccatactgcctccagctgaagctgtagctgaagggagtccctgttccccccaggaaggaggaaacctgagtgacagtggagcccagattccttcccccaccaactgcacccctcttccccaggaaagcagcagcagcagcagcagcaatccaatctaccaagtgctataactcgaggagttgccagccatctgttgtttgcccctcccccgtgccttccttgaccctggaaggtgccactcccactgtcctttcctaataaaatgaggaaattgcatcataacttcgtatagcatacattatacgaacggtaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagt

(19) United States
(12) Patent Application Publication
(10) Pub. No.: US 2023/0220347 A1
(43) Pub. Date: Jul. 13, 2023

(54) SCALING UP MYOGENIC TISSUE: LATE PASSAGE MYOGENICITY

(71) Applicant: Upside Foods, Inc., Berkeley, CA (US)

(72) Inventors: Ara HWANG, Berkeley, CA (US); Daphne Dambournet, Paris (FR); Rachel Valenzuela, Berkeley, CA (US); Robert Anthony Sierra, Berkeley, CA (US)

(21) Appl. No.: 18/069,768

(22) Filed: Dec. 21, 2022

Related U.S. Application Data
(60) Provisional application No. 63/293,578, filed on Dec. 23, 2021.

(51) Int. Cl. C12N 5/077; C12N 5/16; C12N 15/113; C12N 9/12

(52) U.S. Cl. CPC C12N 5/0658; C12N 5/16; C12N 15/113; C12N 9/1276; C12N 2501/16; C12N 2501/155; C12N 2501/415; C12N 2501/065; C12N 2506/1307; C12N 2510/04; C12N 2310/12; C12Y 207/07049

(57) ABSTRACT

The present disclosure relates to methods for improving myogenic differentiation capacity of a cell line or an immortalized cell line. For example, the present disclosure relates to methods of exposing an immortalized cell line (e.g., an immortalized fibroblast cell line) to culture media comprising signaling pathway agonists, antagonist, or a combination thereof in order to improve differentiation capacity. In another example, the present disclosure relates to methods of improving differentiation capacity of a cell line or an immortalized cell line where the method includes transforming an immortalized cell line with one or more myogenic regulatory factors and exposing the immortalized cell line to culture media comprising signaling pathway agonists, antagonists, or a combination thereof.

### 1. CROSS-REFERENCE TO RELATED APPLICATIONS

This application claims the benefit of U.S. Provisional Application No. 63/293,578 filed on Dec. 23, 2021, which is hereby incorporated by reference in its entirety.

### 2. SEQUENCING LISTING

The instant application contains a Sequence Listing, which has been submitted in XML format and is hereby incorporated herein by reference in its entirety. Said XML copy, created on Dec. 17, 2022 is named 54364-US-Sequence-Listing.xml, and is 84.1 kilobytes (KB) in size.

### 3. BACKGROUND OF THE INVENTION

Livestock production systems as we know them today are the result of an interaction between domestic animals and the environment, modulated by human activities, that date back to Neolithic times. Animal agriculture uses more than three-quarters of the world's agricultural land (Foley et al. 2011; Poore and Nemecek 2018). As a result, the impact of agriculture, forestry and related land use is extensive and far reaching. It accounts for 18.4% of global greenhouse gas emissions and animal farming quite possibly may be the largest human-related source of water pollution.

The challenges from climate change and the consequences on food security and the agriculture socio-economy will vary across the globe, the consensus is that it will have profound impact on world hunger and access to animal protein. Rising temperatures, drought and increase in frequency of extreme weather events will greatly affect available land for agriculture, irrigation, and supply chains to further drive the cost of already expensive sources of animal protein even higher.

Livestock farming has an immense carbon footprint and deleterious effects on climate change from deforestation to transport, waste management to food storage, each step of the food chain brings with it a high carbon footprint. Feeding the billions of people every day is a massive task and one that is likely to get even greater as human populations rise and we increasingly feel the effects of climate change.

If the world is to meet the ambition of reaching net zero carbon emissions by the middle of the century as outlined in the Paris Agreement on climate change, the food industry will have to play its part. All this and more clearly indicates that new ways to provide protein sources without needing to rear, slaughter and butcher livestock which not only reduce the impact of livestock farming on our environment but are also insulated from the effects of climate change are highly important.

### 4. SUMMARY OF THE INVENTION

This disclosure features methods for improving myogenic differentiation capacity in a cell line or an immortalized cell line. Improving differentiation capacity of a cell line or an immortalized cell line is important because cell line adaptation into desired media (e.g., low cost media) and culture format (i.e., suspension culture) takes long periods (e.g., 1-3 months). This cell line adaptation is important for enabling production of cell-based meat from cell lines, in particular, from immortalized cell lines. Once a cell line or an immortalized cell line is generated and deemed capable of being used at commercial scale to generate sufficient cells to make cell-based meat, this cell line or immortalized cell line is required to maintain its growth and function for long periods (e.g., 1-6 months) in a bioreactor. As shown in FIGS. 1A-1D, culturing immortalized cell lines for the number of population doubling levels (e.g., about 100 PDLs) required for full cell line adaption results in loss of myogenic differentiation capacity (e.g., decreased Pax7 expression, decreased MyHC1 expression, and decreased ability to form myotubes). The methods and compositions provided herein improve the myogenic differentiation capacity of a cell line or an immortalized cell line, thereby enabling the production of a cell-based meat product from cell lines and immortalized cells that may have otherwise lost their myogenic differentiation capacity during the requisite cell line adaptation steps.

This disclosure is based in part on the discovery that culturing cell lines or immortalized cell lines in culture medium comprising at least a first Activin A inhibitor, at least a first BMP inhibitor, at least a first WNT activator, at least one epigenetic modulator, or a combination thereof, increases the myogenic differentiation capacity of the cell line or immortalized cell line. This disclosure is also based, in part, on the discovery that introducing into, or incorporating into the genome of, a cell of the cell line or immortalized cell line a polynucleotide encoding at least a first myogenic regulatory factor polypeptide also increased myogenic differentiation capacity of the cell line or the immortalized cell line. In combining these two discoveries (i.e., culturing the cell lines transformed with a polynucleotide encoding at least a first myogenic regulatory factor polypeptide in culture medium comprising at least a first Activin A inhibitor, at least a first BMP inhibitor, at least a first WNT activator, at least one epigenetic modulator, or a combination thereof), the inventors discovered a synergistic effect resulting in robust improvement in the myogenic differentiation capacity of the immortalized cell lines.

Overall, this work demonstrated the ability to improve myogenic differentiation capacity of late passage immortalized cell lines that lose or have lost their myogenic differentiation capacity following extended culture periods. The extended culture periods typically required for cell line adaption into particular culture formats and cell culture media, for growing large quantities of cells from a small tissue sample without the need of getting new biopsies from animals, and for ensuring that the ultimate cell based meat product has a consistent flavor by, for example, having the entire product line made from clones of a single cell line. Therefore, this disclosure is especially powerful as it provides (1) a method for ensuring myogenic differentiation capacity is not lost despite the extended culture periods and/or (2) a method for restoring (i.e., improving) the myogenic differentiation capacity if it is reduced or lost during the extended culture periods.

In one aspect, this disclosure features a method for improving myogenic differentiation capacity of a cell line, the method comprising:
(a) contacting the cell line with a culture media comprising:
   (i) at least a first Activin A inhibitor,
   (ii) at least a first BMP inhibitor, or
   (iii) at least a first WNT activator, or a combination thereof,
(b) inducing myogenic specific differentiation, comprising inducing formation of myocytes, myoblasts, myotubes, or a combination thereof,
thereby improving the cell line's myogenic differentiation capacity as compared to a control.

In some embodiments, the method also includes, prior to step (a), isolating a population of cells from skin or muscle tissue to form a cell line.

In some embodiments, the method also includes immortalizing the cell line, wherein the immortalizing step comprises introducing into, or incorporating into the genome of, a cell of the cell line a polynucleotide encoding a telomerase reverse transcriptase (TERT) polypeptide, thereby generating an immortalized cell line.

In some embodiments, the cell line is a late passage cell line.

In some embodiments, the late passage cell line has lost myogenic differentiation capacity.

In some embodiments, the method also includes introducing into, or incorporating into the genome of, a cell of the cell line a polynucleotide encoding at least a first myogenic regulatory factor polypeptide, wherein the at least first myogenic regulatory factor is selected from: MYOD, MYOG, MEF2B, PAX7, PAX3, and PITX1.

In some embodiments, the polynucleotide comprising the first myogenic regulatory factor polypeptide further comprises a nucleic acid sequence encoding a second myogenic regulatory factor polypeptide, a nucleic acid sequence encoding a third myogenic regulatory factor polypeptide, or a combination thereof, wherein the first myogenic regulatory factor polypeptide is a PAX7 polypeptide or a fragment thereof, the second myogenic regulatory factor polypeptide is a MEF2B polypeptide or a fragment thereof, and the third myogenic regulatory factor polypeptide is a MYOD polypeptide or a fragment thereof).

In some embodiments, the Activin A inhibitor is selected from: A-83-01, E-616542, SB431542, TGFβRI-IN-3, R-268712, Follistatin, and Follistatin-like-3.

In some embodiments, the BMP inhibitor is selected from: LDN193189, Noggin, Chrodin, and Gremlin.

In some embodiments, the WNT activator is selected from: CHIR99021, BIO, AZD1080, WNT1a, WNT3a, WNT4, and WNT7.

In some embodiments, the method also includes contacting the cell line with a culture media comprising a histone deacetylase inhibitor.

In some embodiments, the cell line is derived from a species selected from: poultry, livestock, game, or aquatic animal species.

In some embodiments, the cell line is a fibroblast cell line.

In some embodiments, the cell line is not a pluripotent stem cell line.

In some embodiments, inducing myogenic-specific differentiation comprises contacting the cell line with a differentiation medium.

In another aspect, this disclosure features a method for restoring myogenic differentiation capacity of a late passage cell line, the method comprising:
(a) contacting the late passage cell line with a culture media comprising:
   (i) at least a first Activin A inhibitor,
   (ii) at least a first BMP inhibitor, or
   (iii) at least a first WNT activator, or a combination thereof; and
(b) inducing myogenic specific differentiation, comprising inducing formation of myocytes, myoblasts, myotubes, or a combination thereof,
thereby restoring the late passage cell line's myogenic differentiation capacity as compared to a late passage control cell line.

In some embodiments, the late passage cell line has exceeded 60 population doublings.

In some embodiments, the late passage control cell line has lost myogenic differentiation capacity at or above 60 population doublings.

In another aspect, this disclosure features a population of myocytes, myoblasts, myotubes, multinucleated myotubes, satellite cells, skeletal muscle fibers, or any combination thereof produced by any of the methods described herein.

In another aspect, this disclosure features an in vitro method for producing a cell-based meat product, the method comprising: forming the myocytes, myoblasts, myotubes, or a combination thereof, into a cell based meat product.

### 5. BRIEF DESCRIPTION OF THE DRAWINGS

The patent or application file contains at least one drawing executed in color. Copies of this patent or patent application publication in color drawing(s) will be provided by the Office upon request and payment of the necessary fee.

These and other features, aspects, and advantages of the present invention will become better understood with regard to the following description, and accompanying drawings, where:

FIG. 1A-D shows immunofluorescence images of TERT-immortalized myoblasts having lost their myogenic differentiation capacity. FIG. 1A shows primary chicken myoblasts express high Pax7 levels. FIG. 1B indicates primary chicken myoblasts expressing myosin heavy chain (MyHC). FIG. 1C shows that TERT-immortalized myoblasts displayed almost no Pax7 signal. FIG. 1D indicates almost no myotube formation for.

FIG. 2 shows a three-factor media panel design targeting 3 pathways crucial for stem cell biology. “+” indicates activation of the respective signaling pathway. “−” indicates inhibition, interference, or a block of the respective signaling pathway.

FIG. 3 is a bar graph showing percentage of Pax7 positive cells for each of the conditions tested in the factorial media panel described in FIG. 2. FIG. 3 identifies components that enhance percentage of Pax7 positive cells in an immortalized cell line.

FIG. 4A-F displays cell population indicating elevated number of cells expressing myogenic progenitor marker Pax7 after media 1 and 9 treatments. FIG. 4A displays control cells. FIG. 4B shows presence of Pax7 cells after treatment with media 1. FIG. 4C shows cells treated with media 9. FIG. 4D displays control media. FIG. 4E shows increased cellular expression of Pax7 positive cells after media 1 treatment. FIG. 4F shows increased cellular expression of Pax7 positive cells after media 9 treatment. Arrows point to representative Pax7 positive cells.

FIG. 5 is a bar graph showing % Myosin Heavy Chain (MyHC) area for each of the conditions tested in the full factorial media panel as described in FIG. 2. FIG. 5 identifies components that enhance percent of cell area that express myogenic differentiation marker, myosin heavy chain in an immortalized cell line.

FIG. 6A-C shows representative images of myotube formation in cells in response to specific media treatment. FIG. 6A shows cells subjected to control media. FIG. 6B shows cells contacted with ME9. FIG. 6C shows cells contacted with ME17.

FIG. 7A-C shows representative images of myotube formation in 7A primary cells stably transfected with a vector containing a polynucleotide encoding ggMyoD. FIG. 7A is a positive control. FIG. 7B is a negative control. FIG. 7C shows formation of thin myotubes after 7 days post-differentiation. Abbreviation: gg=Gallus gallus; MyoD=myosin D.

FIG. 8 shows expression of downstream myogenic factors in 7A primary chicken fibroblasts following transformation with ggMyoD mRNA. Abbreviation: Myf6=myogenic factor 6; MyoD=myosin D; MyoG=myogenin; MYMK=myomarker, myoblast fusion factor; MyHC1e=myosin heavy chain 1E.

FIG. 9 shows expression of downstream myogenic factors in 1A primary chicken fibroblasts following transformation with ggMyoD.

FIG. 10A-C shows representative images of immortal myoblast cell lines following transformation with a polynucleotide encoding Pax7, MEF2b, and MyoD (“7MM”). FIG. 10A indicates that overexpression of MyoD delayed loss of myogenicity in small molecules cocktail (M9). FIG. 10B also indicates that overexpression of Pax7/MEF2b/MyoD delayed loss of myogenicity with ME9. FIG. 10C shows representative images of the control.

FIG. 11 shows expression of endogenous downstream myogenic factors in in TERT-immortalized 7A chicken fibroblasts having a PDL of about 40 following transformation with ggMyoD mRNA. FIG. 11 shows that transformation of ggMyoD mRNA can induce the expression of downstream myogenic factors in an earlier passage of TERT-immortalized 7A chicken fibroblasts (PDL˜40). Abbreviation: Myf6=myogenic factor 6; MyoD=myosin D; MyoG=myogenin; MYMK=myomarker, myoblast fusion factor; MyHC1e=myosin heavy chain 1E.

FIG. 12 shows expression of endogenous downstream myogenic factors in 7A chicken fibroblasts and 7A chicken primary cells following transformation with ggMyoD.

FIG. 13A-C shows representative images of MyHC staining and myotube formation in old 7A TERT cells following transformation with ggMyoD. FIGS. 13A-13C show that transforming old 7A TERT cells is not sufficient to induce myotube formation. FIG. 13A shows 7A TERT ggMyoD cells in ME58. FIG. 13B shows 7A TERT ggMyoD cells suspended in ME9. FIG. 13C shows cells suspended in ME9 in the presence of sodium butyrate.

FIG. 14A-H shows representative images of MyHC staining and myotube formation in 7A TERT fibroblasts. FIG. 14A shows non-transformed 7A TERT fibroblasts grown in ME58 media. FIG. 14E shows non-transformed 7A TERT fibroblasts grown in ME9 media. FIG. 14B shows 7A TERT fibroblasts transformed with a polynucleotide encoding MYOD and grown in ME58 media. FIG. 14F shows 7A TERT fibroblasts transformed with a polynucleotide encoding MYOD and grown in ME9 media. FIG. 14C shows 7A TERT fibroblasts transformed with a polynucleotide encoding PAX7, MEF2B, and MYOD and grown in ME58 media. FIG. 14G shows 7A TERT fibroblasts transformed with a polynucleotide encoding PAX7, MEF2, and MYOD and grown in ME9 media. FIG. 14D shows non-transformed 8D fibroblasts transformed grown in ME58 media. FIG. 14H shows non-transformed 8D fibroblasts grown in ME9 media.

FIG. 15 shows a histogram of RNA expression levels of MyoD in immortalized Bovine taurus (bt) fibroblasts transfected with a polynucleotide encoding btMyoD (“8G TCC+MyoD”) compared to the non-transfected control (8G TCC).

FIG. 16A-16B shows representative images of MyHC staining of 8G bovine TERT fibroblast. FIG. 16A and FIG. 16B show that 8G bovine TERT fibroblasts transdifferentiate into myoblasts and myotubes formation as indicated by tubes staining positive for myosin heavy chain.

### 6. DETAILED DESCRIPTION OF THE INVENTION

This disclosure features methods for improving myogenic differentiation capacity in immortalized cells lines to enable cell line adaption without compromising necessary myogenic differentiation capacity in late passage cells (e.g., immortalized cells or cells that have exceed about 60 population doublings). In particular, this disclosure is based in part on the discovery that culturing immortalized cell lines in culture medium comprising at least a first Activin A inhibitor, at least a first BMP inhibitor, at least a first WNT activator, or a combination thereof increases the myogenic differentiation capacity (e.g., increased percentage of Pax7 positive cells as compared to a control or increased percentage of MyHC area as compared to a control) of the cell line. The Applicants also discovered that transforming immortalized cell lines with a polynucleotide encoding at least a first myogenic regulatory factor polypeptide also increased myogenic differentiation capacity of the immortalized cell lines. Furthermore, in combining these two discoveries (i.e., culturing the immortalized cell lines transformed with the polynucleotide encoding the at least first myogenic regulatory factor polypeptide in a culture medium comprising the at least first Activin A inhibitor, the at least first BMP inhibitor, and the at least first WNT activator), a synergistic effect was discovered resulting in robust improvement in the myogenic differentiation capacity of the immortalized cell lines.

### 6.1. Definitions

Unless defined otherwise, all technical and scientific terms used herein have the same meaning as is commonly understood by one of ordinary skill in the art to which this disclosure belongs. In the event that there is a plurality of definitions for terms cited herein, those in this section prevail unless otherwise stated.

Throughout this disclosure, the term “a” or “an” entity refers to one or more of that entity. As such, the terms “a” (or “an”), “one or more,” and “at least one” can be used interchangeably herein. Furthermore, “and/or” as used in a phase such as “A and/or B” herein is intended to include “A and B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “comprising” and “including” or grammatical variants thereof are to be taken as specifying the stated features, integers, steps, or components but do not preclude the addition of one or more additional features, integers, steps, components, or groups thereof. This term encompasses the terms “consisting of” and “consisting essentially of”.

As used herein, the terms “cell” and “cell line” are sometimes used interchangeably. As used herein, the term “cell” can refer to one or more cells originating from a cell line. As used herein, the term “cell line” can refer to a population of cells.

As used herein, the term “skeletal muscle progenitor cell” (SMPC) refers to a stem cell that is SMPCs are also referred to herein as myogenic progenitors.

As used herein, the term “myoblast” refers to mononucleated muscle cells that are in proliferating state. They are embryonic precursors of myocytes, also called muscle cells. Although myoblasts may be classified as skeletal muscle myoblasts, smooth muscle myoblasts, and cardiac muscle myoblasts depending on the type of muscle cell that they will differentiate into, in this specification the term myoblasts refer to skeletal muscle myoblasts.

As used herein, the term “myotube” refers to elongated structures, the result of differentiated myoblast. Upon differentiation, myoblasts fuse into one or more nucleated myotubes and express skeletal muscle markers.

As used herein, the term “immortalized cell” refers to cells that are passaged or modified to proliferate indefinitely and evade normal cellular senescence.

As used herein, the term “population doubling level (PDL)” refers to the total number of times the cells in the population have doubled since their primary isolation in vitro. Mathematically this is described as n=3.32 (log UCY−log 1)+X, where n=the final PDL number at end of a given subculture, UCY=the cell yield at that point, 1=the cell number used as inoculum to begin that subculture, and X=the doubling level of the inoculum used to initiate the subculture being quantitated.

As used herein the term “passaged cell” refers to the number of times the cells in the culture have been subcultured. This may occur without consideration of the inoculation densities or recoveries involved.

As used herein, the term “myogenic differentiation capacity” refers to a cells ability to differentiate to a myogenic cell and/or an increase of one or more markers. Non-limiting examples of a myogenic cell include: myoblasts, myocytes, myotubes, satellite cells, side population cells, muscle derived stem cells, mesenchymal stem cells, myogenic pericytes, or mesoangioblasts. Myogenic differentiation capacity can be measured according to the methods described herein.

As used herein, the term “transdifferentiation” refers to the conversion of a cell type present in one tissue or organ into a cell type from another tissue or organ without going through a pluripotent cell state. Transdifferentiation between some cell types can occur naturally. In other cases, transdifferentiation can be induced using exogenous factors. Non-limiting examples of exogenous factors used for transdifferentiation include small molecules, growth factors, and/or genetic engineering.

As used herein, the terms “transformed,” “transduced,” and “transfected” are used interchangeably unless otherwise noted. Each term refers to the introduction of a nucleic acid sequence or polypeptide into a cell (e.g., an immortalized cell).

### 6.2. Methods for Improving Differentiation Capacity

This disclosure features methods for improving myogenic differentiation capacity of a cell line, a late passage cell line, or an immortalized cell line. Applying the methods described herein to a cell line or an immortalized cell line results in the cell line being better suited to produce the cell types of interest, for example, cell types used for cultured food production, including myocytes, myoblasts, myotubes, multinucleated myotubes, satellite cells, skeletal muscle fibers, or any combination thereof. Other methods for improving differentiation potential for self-renewing cells lines (e.g., embryonic stem cells, induced pluripotent stem cells, and extraembryonic cell lines) are as described in WO2015066377A1, which is herein incorporated by reference in its entirety.

In some embodiments, differentiation capacity refers to the ability of a cell line to differentiate into a cell type of interest (e.g., any of the cell types of interest described herein). In some embodiments, the cell type of interest includes myocytes, myoblasts, myotubes, multinucleated myotubes, satellite cells, skeletal muscle fibers, or any combination thereof. In one embodiment, myogenic differentiation capacity refers to the ability of a cell line to differentiate into a myogenic cell (e.g., myocytes, myoblasts, myotubes, multinucleated myotubes, satellite cells, skeletal muscle fibers, or any combination thereof). In some embodiments, myogenic differentiation refers to a cell that differentiates by expressing one or more phenotypes characteristic of differentiated, e.g. terminally differentiated, myotubes. In some embodiments, a method for improving differentiation capacity (e.g., myogenic differentiation capacity) includes contacting a cell line, a late passage cell line, or an immortalized cell line (e.g., an immortalized fibroblast cell line) with culture media comprising signaling pathway agonists, antagonist, or a combination thereof. In another embodiment, a method for improving differentiation capacity (e.g., myogenic differentiation capacity) includes introducing into, or incorporating into the genome of, a cell of the cell line (e.g., a late passage cell line) or immortalized cell line a polynucleotide encoding at least a first myogenic regulatory factor polypeptide. In yet another embodiment, a method of improving differentiation capacity (e.g., myogenic differentiation capacity) of a cell line or an immortalized cell line includes introducing into, or incorporating into the genome of, a cell of the cell line or immortalized cell line a polynucleotide encoding at least a first myogenic regulatory factor polypeptide and contacting the cell line or immortalized cell line to culture media comprising signaling pathway agonists, antagonists, or a combination thereof.

In some embodiments a late passage cell line is a cell line that exceed 40, 41, 42, 43, 44, 45, 46, 47, 48, 49, 50, 51, 52, 53, 54, 55, 56, 57, 58, 59, or 60, or more population doublings.

In some embodiments, as a result of the methods provided herein, the cell line, the late passage cell line, or the immortalized cell line (e.g., immortalized fibroblast cell line) exhibits an increased differentiation capacity (e.g., myogenic differentiation capacity) as compared to a cell line or an immortalized cell line not subjected to the methods described herein. For example, as a result of the methods provided herein, the cell line or immortalized cell line exhibits an increased myogenic differentiation capacity as compared to an immortalized cell line not exposed to: (i) at least a first Activin A/TGF-β inhibitor and at least a first BMP inhibitor; (iii) at least a first Activin A/TGF-β inhibitor, at least a first BMP inhibitor, and at least a first WNT activator, (iii) at least a first Activin A/TGF-β inhibitor, at least a first BMP inhibitor, and one or more myogenic regulatory factor polypeptides; or (iv) at least a first Activin A/TGF-β inhibitor, at least a first BMP inhibitor, at least a first WNT activator, and one or more myogenic regulatory factor polypeptides.

In some embodiments, as a result of the methods provided herein, the cell line, the late passage cell line, or the immortalized cell line (e.g., immortalized fibroblast cell line) exhibits an increased myogenic differentiation capacity as compared to any reference strain, including other immortalized cell lines.

In some embodiments, as a result of the methods provided herein, the immortalized cell line (e.g., immortalized fibroblast cell line) can transdifferentiate into a cell type of interest (e.g., a myoblast).

In some embodiments, prior to exposing the immortalized cell line to at least a first Activin A inhibitor, at least a first BMP inhibitor, at least a first WNT activator, at least a first myogenic regulatory factor polypeptide, an epigenetic modulator, or a combination thereof, the cell line or immortalized cell line had a population doubling level (PDL) of at least 60 ((e.g., at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more passages).

In some embodiments, prior to exposing the cell line, the late passage cell line, or the immortalized cell line to at least a first Activin A inhibitor, at least a first BMP inhibitor, at least a first WNT activator, at least a first myogenic regulatory factor polypeptide, an epigenetic modulator, or a combination thereof, the cell line or immortalized cell had less than 5% Pax7⁺ cells (e.g., less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1%).

In some embodiments, prior to exposing the cell line (e.g., the late passage cell line) or the immortalized cell line to at least a first Activin A inhibitor, at least a first BMP inhibitor, at least a first WNT activator, at least a first myogenic regulatory factor polypeptide, an epigenetic modulator, or a combination thereof, the cell line or immortalized cell had less than 5% MyHC1⁺ cells (e.g., less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1%).

In some embodiments, prior to exposing the cell line (e.g., the late passage cell line) or the immortalized cell line to at least a first Activin A inhibitor, at least a first BMP inhibitor, at least a first WNT activator, at least a first myogenic regulatory factor polypeptide, or a combination thereof, the cell line or immortalized cell lacks the ability to form myotubes.

In some embodiments, increased differentiation capacity (e.g., myogenic differentiation capacity) is achieved even after the cell line or immortalized cell line is cultured for at least 60 passages (e.g., at least 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 passages).

In some embodiments, differentiation capacity (e.g., myogenic differentiation capacity of a cell line or an immortalized cell line (e.g., an immortalized fibroblast cell line) is measured by determining the increase in percentage of cells that differentiate into a cell type of interest (e.g., myogenic cell) as compared to a cell line or an immortalized cell line not subjected to the methods described herein. In some embodiments, differentiation capacity (e.g., myogenic differentiation capacity) of a cell line or an immortalized cell line is measured by determining the increase in the total number of cells that differentiate into a cell type of interest (e.g., myogenic cell) as compared to a cell line or an immortalized cell not subjected to the methods described herein. Non-limiting examples of cell types of interest (e.g., cell types produced when the cell line or the immortalized cell line is exposed to differentiation media) includes: myocytes, myoblasts, myotubes, multinucleated myotubes, satellite cells, or skeletal muscle fibers, or any combination thereof.

In some embodiments, myogenic differentiation capacity of a cell line (e.g., a late passage cell line) or an immortalized cell line is determined by measuring paired box 7 (Pax7) expression (e.g., Pax7 RNA or protein). For example, an increase in the percentage of cell or immortalized cells that are Pax7⁺ (Pax7 positive) as compared to a control indicates increased myogenic differentiation capacity of the immortalized cell line.

In some embodiments, myogenic differentiation capacity of a cell line (e.g., a late passage cell line) or an immortalized cell line is determined by measuring myosin heavy chain 1 (MyHC1) expression (e.g., MyHC1 RNA or protein). For example, an increase in the percentage of cell or immortalized cells that are MyHC1⁺ (MyHC1 positive) as compared to a control indicates increased myogenic differentiation capacity of the immortalized cell line.

In some embodiments, myogenic differentiation capacity of a cell line (e.g., a late passage cell line) or an immortalized cell line is determined by measuring myotube formation. For example, an increase in the ability to form myotubes as compared to a control indicates increased myogenic differentiation capacity of the immortalized cell line.

In some embodiments, myogenic differentiation capacity of a cell line (e.g., a late passage cell line) or an immortalized cell line is determined by measuring the number or percentage (of the total population) of myocytes, myoblasts, myotubes, or a combination thereof. For example, an increase in the number or percentage of myocytes, myoblasts, myotubes, cells expressing differentiated myogenic cell phenotypes, or a combination thereof as compared to a control indicates increased myogenic differentiation capacity of the cell line or immortalized cell line.

In some embodiments, myogenic differentiation capacity of a cell line (e.g., a late passage cell line) or an immortalized cell line is determined by measuring myogenin (MyoG) expression (e.g., MyoG RNA). For example, an increase in the percentage of immortalized cells that are MyoG⁺ (MyoG positive) as compared to a control indicates increased myogenic differentiation capacity of the cell line or the immortalized cell line.

In some embodiments, myogenic differentiation capacity of a cell line (e.g., a late passage cell line) or an immortalized cell line is determined by measuring Myomaker (Mymk) expression (e.g., MYMK RNA). For example, an increase in the percentage of immortalized cells that are MYMK⁺ (MYMK positive) as compared to a control indicates increased myogenic differentiation capacity of the cell line or the immortalized cell line.

### 6.2.1. Culture Methods for Improving Differentiation Capacity

This disclosure features methods of improving differentiation capacity (e.g., myogenic differentiation capacity) of an immortalized cell line (e.g., an immortalized fibroblast cell line) using culture media comprising one or more signaling pathway agonists, antagonists, or a combination thereof. For example, a method for improving differentiation capacity (e.g., myogenic differentiation capacity) of an immortalized cell line comprises exposing the cell line to at least a first Activin A inhibitor; at least a first BMP inhibitor; at least a first WNT activator, or a combination thereof. In some embodiments, the culture medium is used in combination with genetic engineering (e.g., engineering cells to express at least a first myogenic regulatory factor polypeptide) to improve differentiation capacity of an immortalized cell line.

This disclosure also features methods of improving differentiation capacity (e.g., myogenic differentiation capacity) of a cell line (e.g., a late passage cell line) using culture media comprising one or more signaling pathway agonists, antagonists, or a combination thereof. For example, a method for improving differentiation capacity (e.g., myogenic differentiation capacity) of a cell line comprises exposing the cell line to at least a first Activin A inhibitor; at least a first BMP inhibitor; at least a first WNT activator, or a combination thereof. In some embodiments, the culture medium is used in combination with genetic engineering (e.g., engineering cells to express at least a first myogenic regulatory factor polypeptide) to improve myogenic differentiation capacity of an immortalized cell line.

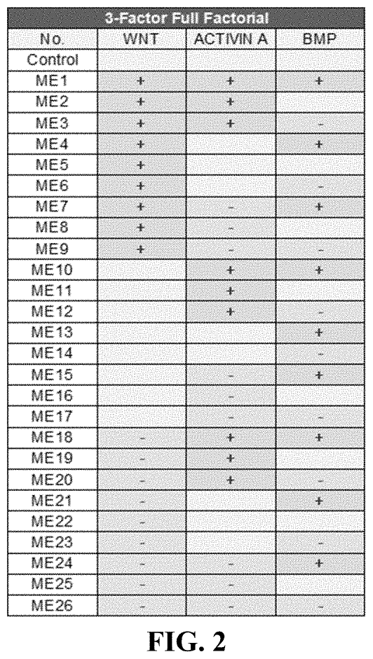

In some embodiments, a method for improving myogenic differentiation capacity of a cell line or an immortalized cell line comprises culturing the cell line in a culture media (e.g. a proliferation media) as described, for example, in FIG. 2. As shown in FIG. 2, WNT, TGF (Activin A), and BMP signaling pathways were activated (e.g., using CHIR99021 (5 μM), Activin A (25 ng/mL), or BMP4 (10 ng/mL), respectively) or inhibited (e.g., using IWR1 (2.5 μM), A-83-01 (5 μM), or LDN193189 (0.4 μM), respectively). In some embodiments, a full factorial design was used to generate 27 combinations of media, including a control that had no small molecules or growth factors added to the base media.

In some embodiments, the method for improving myogenic differentiation capacity of a cell line (e.g., a late passage cell line) or an immortalized cell line comprises culturing the cell line or immortalized cell line in a culture media comprising an Activin A inhibitor (e.g., A-83-01 or Follistatin), and a BMP inhibitor (e.g., LDN193189 or Noggin), or any combination thereof.

In some embodiments, the method for improving myogenic differentiation capacity of a cell line (e.g., a late passage cell line) or an immortalized cell line comprises culturing the cell line or immortalized cell line in a culture media comprising a WNT activator (e.g., WNT1a), an Activin A inhibitor (e.g., Follistatin), and a BMP inhibitor (e.g., Noggin).

In some embodiments, base media includes 20% fetal bovine serum (FBS), fibroblast growth factor 2 (FGF2), 2% chicken serum, and DMEM/F12. Other non-limiting examples of base media include: 10% FBS, FGF2, 2% chicken serum, and DMEM/F12; 20% FBS, FGF2, 2% chicken serum, and DMEM; or 10% FBS, FGF2, 2% chicken serum, and DMEM.

In some embodiments, base media includes serum (e.g., bovine serum, chicken serum or horse serum, or a combination thereof). For example, base media includes about 10% serum, 11% serum, about 12% serum, about 13% serum, about 14% serum, about 15% serum, about 16% serum, about 17% serum, about 18% serum, about 19%, or about 20% serum. In some embodiments, base media includes about 20% serum (e.g., bovine serum, chicken serum, or horse serum, or a combination thereof). In some embodiments, serum is horse serum. In such cases, the base media comprises about 1% horse serum, about 2% horse serum, about 3% horse serum, about 4% horse serum, or about 5% horse serum.

In some embodiments, base media includes fibroblast growth factor 2 (FGF2) (e.g., recombinant FGF2 (R&D Systems)). For example, base media includes about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL or more of FGF2. In some embodiments, base media includes about 10 ng/mL FGF2.

In some embodiments, the methods described herein include contacting the cell line (e.g., the late passage cell line) or the immortalized cell line with a proliferation media. In such cases, the proliferation media includes base media and one or more additional components. In some embodiments, proliferation media includes 10% fetal bovine serum (FBS), fibroblast growth factor 2 (FGF2), 2% chicken serum, and DMEM/F12. In some embodiments, proliferation media includes serum (e.g., bovine serum, chicken serum, or horse serum, or a combination thereof). For example, base media includes about 5% serum, about 6% serum, about 7% serum, about 8% serum, about 9% serum, about 10% serum, about 11% serum, about 12% serum, about 13% serum, about 14% serum, or about 15% serum. In some embodiments, proliferation media includes about 10% serum (e.g., bovine serum, chicken serum, or horse serum, or a combination thereof).

In some embodiments, a method for improving myogenic differentiation capacity of a cell line (e.g., a late passage cell line) or an immortalized cell line comprises culturing the cell line in a culture media (e.g., a proliferation media) as described, for example, in FIG. 2. In some embodiments, the culture media (e.g., the proliferation media) is selected from ME1, ME2, ME3, ME4, ME5, ME6, ME7, ME8, ME9, ME10, ME11, ME12, ME13, ME14, ME15, ME16, ME17, ME18, ME19, ME20, ME21, ME22, ME23, ME24, ME25, or ME26. In some embodiments, the proliferation media is ME9. In some embodiments, the proliferation media is ME17. In some embodiments, the proliferation media is ME1. In some embodiments, the proliferation media is ME2. In some embodiments, the proliferation media is ME3. In some embodiments, the proliferation media is ME5. In some embodiments, the proliferation media is ME6. In some embodiments, the proliferation media is ME7. In some embodiments, the proliferation media is ME8.

In some embodiments, the proliferation media is ME9. ME9 comprises DMEM/F12, about 20% FBS, about 5% chicken serum, CHIR99021, A-83-01, and LDN193189.

In some embodiments, culture media (e.g., base media, proliferation media, and differentiation media) is as described in WO 2021/248141, which is herein incorporated by reference in its entirety.

In some embodiments, exposing the cell line or immortalized cell line to at least a first Activin A/TGF-β inhibitor; at least a first BMP inhibitor; at least a first WNT activator, or a combination thereof is performed for a first period of time. As used herein the phrase “a first period of time” includes any period of time sufficient to allow for improved differentiation capacity in the immortalized cell line. For example, a first period of time includes from about 12 hours to about 72 hours (e.g., about 12 hours to about 68 hours, about 12 hours to about 64 hours, about 12 hours to about 60 hours, about 12 hours to about 56 hours, about 12 hours to about 52 hours, about 12 hours to about 48 hours, about 12 hours to about 44 hours, about 12 hours to about 40 hours, about 12 hours to about 36 hours, about 12 hours to about 32 hours, about 12 hours to about 28 hours, about 12 hours to about 24 hours, about 12 hours to about 20 hours, about 12 hours to about 16 hours, about 16 hours to about 72 hours, about 16 hours to about 68 hours, about 16 hours to about 64 hours, about 16 hours to about 60 hours, about 16 hours to about 56 hours, about 16 hours to about 52 hours, about 16 hours to about 48 hours, about 16 hours to about 44 hours, about 16 hours to about 40 hours, about 16 hours to about 36 hours, about 16 hours to about 32 hours, about 16 hours to about 28 hours, about 16 hours to about 24 hours, about 16 hours to about 20 hours, about 20 hours to about 68 hours, about 20 hours to about 60 hours, about 20 hours to about 56 hours, about 20 hours to about 52 hours, about 20 hours to about 48 hours, about 20 hours to about 44 hours, about 20 hours to about 40 hours, about 20 hours to about 36 hours, about 20 hours to about 32 hours, about 20 hours to about 28 hours, about 20 hours to about 24 hours, about 24 hours to about 64 hours, about 24 hours to about 60 hours, about 24 hours to about 56 hours, about 24 hours to about 52, about 24 hours to about 48 hours, about 24 hours to about 44 hours, about 24 hours to about 40 hours, about 24 hours to about 36 hours, about 24 hours to about 32 hours, about 24 hours to about 28 hours, about 28 hours to about 60 hours, about 28 hours to about 56 hours, about 28 hours to about 52 hours, about 28 hours to about 48 hours, about 28 hours to about 44 hours, about 28 hours to about 40 hours, about 28 hours to about 36 hours, about 28 hours to about 32 hours, about 32 hours to about 56 hours, about 32 hours to about 52 hours, about 32 hours to about 48 hours, about 32 hours to about 44 hours, about 32 hours to about 40 hours, about 32 hours to about 36 hours, about 36 hours to about 52 hours, about 36 hours to about 48 hours, about 36 hours to about 44 hours, about 36 hours to about 40 hours, about 40 hours to about 48 hours, about 40 hours to about 44 hours, about 44 hours to about 48 hours, about 48 hours to about 60 hours, about 48 hours to about 72 hours, or about 60 hours to about 72 hours) of culture, where hour 0 is the start of the cell line's or the immortalized cell line's exposure to the Activin A inhibitor; the BMP inhibitor; the WNT activator, or a combination thereof.

In another example, a first period of time includes from about day 1 to about day 14 (e.g., from about day 1 to about day 13, from about day 1 to about day 12, from about day 1 to about day 11, from about day 1 to about day 10, from about day 1 to about day 9, from about day 1 to about day 8, from about day 1 to about day 7, from about day 1 to about day 6, from about day 1 to about day 5, from about day 1 to about day 4, from about day 2 to about day 14, from about day 2 to about day 13, from about day 2 to about day 12, from about day 2 to about day 11, from about day 2 to about day 10, from about day 2 to about day 9, from about day 2 to about day 8, from about day 2 to about day 7, from about day 2 to about day 6, from about day 2 to about day 5, from about day 2 to about day 4, from about day 3 to about day 14, from about day 3 to about day 13, from about day 3 to about day 12, from about day 3 to about day 11, from about day 3 to about day 10, from about day 3 to about day 9, from about day 3 to about day 8, from about day 3 to about day 7, from about day 3 to about day 6, from about day 3 to about day 5, from about day 3 to about day 4, from about day 4 to about day 14, from about day 4 to about day 13, from about day 4 to about day 12, from about day 4 to about day 11, from about day 4 to about day 10, from about day 4 to about day 9, from about day 4 to about day 8, from about day 4 to about day 7, from about day 4 to about day 6, from about day 4 to about day 5, from about day 5 to about day 14, from about day 5 to about day 14, from about day 5 to about day 13, from about day 5 to about day 12, from about day 5 to about day 11, from about day 5 to about day 10, from about day 5 to about day 9, from about day 5 to about day 8, from about day 5 to about day 7, from about day 5 to about day 6, from about day 6 to about day 14, from about day 6 to about day 13, from about day 6 to about day 12, from about day 6 to about day 11, from about day 6 to about day 10, from about day 6 to about day 9, from about day 6 to about day 8, from about day 6 to about day 7, from about day 7 to about day 13, from about day 7 to about day 12, from about day 7 to about day 11, from about day 7 to about day 10, from about day 8 to about day 13, from about day 8 to about day 12, from about day 8 to about day 10, from about day 9 to about day 13, from about day 9 to about day 12, from about day 9 to about day 11, from about day 10 to about day 13, or from about day 11 to about day 13) of culture, where day 0 is the day the cell line's or the immortalized cell line's are contacted with the Activin A/TGF-β inhibitor; the BMP inhibitor; the WNT activator, or any combination thereof.

### 6.2.1.1 Activin A

In some embodiments, the method for improving myogenic differentiation capacity of a cell line (e.g., a late passage cell line) or an immortalized cell line includes modulating Activin A-mediated signaling (Activin A/TGF-β signaling). In some embodiments, modulating Activin-A signaling includes inhibiting, blocking, interfering, or attenuating Activin A signaling using one or more Activin A inhibitory agents. Non-limiting examples of agents that inhibit Activin A activity include: peptide inhibitors, small molecule antagonists, antibodies (or antigen-binding fragments thereof), and/or agents which do not directly bind Activin A or Activin A signaling components but nonetheless interfere with, block or attenuate Activin A-mediated signaling.

In some embodiments, the method includes modulating Activin A-mediated signaling by inhibiting Activin/NODAL/TGF-β signaling. In some embodiments, inhibiting Activin A-mediated signaling includes inhibiting activin receptor-like kinase (ALK), including ALK5 (type I transforming growth factor-β receptor), ALK4 (type IB activin receptor), and ALK7 (type I NODAL receptor).

In some embodiments, the method for improving myogenic differentiation capacity of a cell line (e.g., a late passage cell line) or an immortalized cell line includes contacting the cell line or the immortalized cell line with a culture media comprising at least a first Activin A inhibitor. In some embodiments, the method for improving myogenic differentiation capacity of a cell line or an immortalized cell line includes contacting the cell line or the immortalized cell line with a culture media comprising the first Activin A inhibitor and a second Activin A inhibitor (e.g., any of the Activin A inhibitors described herein).

In some embodiments, the Activin A/TGF-β-mediated signaling inhibitor is selected from: A-83-01, E-616542, SB431542, TGFβRI-IN-3, R-268712, Follistatin, and Follistatin-like-3.

In some embodiments, Activin A/TGF-β-mediated signaling is inhibited using A 83-01 (CAS Number: 909910-43-6). In some embodiments, the methods provided herein include contacting the cell line or the immortalized cell line with a culture media comprising A 83-01 at a concentration ranging from about 2.5 μM to about 10 μM (e.g., about 2.5 μM to about 9 about 2.5 μM to about 8 about 2.5 μM to about 7 about 2.5 μM to about 10 about 2.5 μM to about 6 about 2.5 μM to about 5 about 2.5 μM to about 4 μM, 2.5 μM to about 3 about 3 μM to about 10 about 3 μM to about 9 about 3 μM to about 8 about 3 μM to about 7 about 3 μM to about 6 about 3 μM to about 5 about 3 μM to about 4 about 4 μM to about 10 about 4 μM to about 9 about 4 μM to about 8 about 4 μM to about 7 about 4 μM to about 6 about 4 μM to about 5 about 5 μM to about 10 about 5 μM to about 9 about 5 μM to about 8 about 5 μM to about 7 about 5 μM to about 6 about 6 μM to about 10 about 6 μM to about 9 about 6 μM to about 8 about 6 μM to about 7 about 7 μM to about 10 about 7 μM to about 9 about 7 μM to about 8 about 8 μM to about 10 about 8 μM to about 9 or about 9 μM to about 10 In some embodiments, the method includes contacting the cell line or immortalized cell line with a culture media comprising A 83-01 at a concentration of about 5 μM.

In some embodiments, Activin A/TGF-β-mediated signaling is inhibited using E-616542 (CAS Number: 446859-33-2). In some embodiments, the methods provided herein include contacting the cell line or the immortalized cell line with a culture media comprising E-616542 at a concentration ranging from about 2 μM to about 20 μM (or any of the values or subranges therein). In some embodiments, the methods provided herein include contacting the cell line or the immortalized cell line with a culture media comprising E-616542 at a concentration of about 10 μM.

In some embodiments, Activin A/TGF-β-mediated signaling is inhibited using SB431542 (CAS Number: 301836-41-9). In some embodiments, the methods provided herein include contacting the cell line or the immortalized cell line with a culture media comprising SB431542 at a concentration ranging from about 0.1 μM to about 10 μM (or any of the values or subranges therein). In some embodiments, the methods provided herein include contacting the cell line or the immortalized cell line with a culture media comprising SB431542 at a concentration of about 1 μM.

In some embodiments, Activin A/TGF-β-mediated signaling is inhibited using TGFβRI-IN-3 (CAS Number: 2763602-67-9). In some embodiments, the methods provided herein include contacting the cell line or the immortalized cell line with a culture media comprising TGFβRI-IN-3 at a concentration ranging from about 0.1 μM to about 100 μM (or any of the values or subranges therein). In some embodiments, the methods provided herein include contacting the cell line or the immortalized cell line with a culture media comprising TGFβRI-IN-3 at a concentration of about 1 μM.

In some embodiments, Activin A/TGF-β-mediated signaling is inhibited using R-268712 (CAS Number: 879487-87-3). In some embodiments, the methods provided herein include contacting the cell line or the immortalized cell line with a culture media comprising R-268712 at a concentration ranging from about 0.01 μM to about 10 μM (or any of the values or subranges therein). In some embodiments, the methods provided herein include contacting the cell line or the immortalized cell line with a culture media comprising R-268712 at a concentration of about 0.1 μM.

In some embodiments, the method includes modulating Activin A-mediated signaling using an agent that binds to Activin A. In some embodiments, the agent that binds to Activin A is an Activin A binding protein. Non-limiting examples of Activin A binding proteins include Follistatin and Follistatin-like-3. In some embodiments, the methods provided herein include contacting the cell line immortalized cell line with a culture media comprising Follistatin at a concentration of 2.5 ng/mL, about 5 ng/mL, about 7.5 ng/mL about 10 ng/mL, about 12.5 ng/mL, about 15 ng/mL, about 17.5 ng/mL, about 20 ng/mL, about 22.5 ng/mL, about 25 ng/mL, about 27.5 ng/mL, about 30 ng/mL, about 32.5 ng/mL, about 35 ng/mL, about 37.5 ng/mL, about 40 ng/mL, about 42.5 ng/mL, about 45 ng/mL, about 47.5 ng/mL, about 50 ng/mL, about 55 ng/mL about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, or about 200 ng/mL, about 300 ng/mL, about 400 ng/mL, about 500 ng/mL, about 600 ng/mL, about 700 ng/mL, about 800 ng/mL, about 900 ng/mL, or about 1000 ng/mL.

In some embodiments, modulating Activin A signaling includes activating, stabilizing, or inducing Activin A signaling using one or more Activin A activation agents. Non-limiting examples of agents that activate Activin A signaling include: Activin A, TGF beta, and Myostatin. In some embodiments, the method includes contacting the cell line or the immortalized cell line with a culture media comprising Activin A at a concentration of about 2.5 ng/mL, about 5 ng/mL, about 7.5 ng/mL about 10 ng/mL, about 12.5 ng/mL, about 15 ng/mL, about 17.5 ng/mL, about 20 ng/mL, about 22.5 ng/mL, about 25 ng/mL, about 27.5 ng/mL, about 30 ng/mL, about 32.5 ng/mL, about 35 ng/mL, about 37.5 ng/mL, about 40 ng/mL, about 42.5 ng/mL, about 45 ng/mL, about 47.5 ng/mL, about 50 ng/mL, about 55 ng/mL about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, or about 200 ng/mL. In some embodiments, the method includes contacting the cell line or immortalized cell line with a culture media comprising Activin A at about 25 ng/mL.

### 6.2.1.2 BMP

In some embodiments, the method for improving myogenic differentiation capacity of a cell line (e.g., a late passage cell line) or an immortalized cell line includes modulating BMP-mediated signaling (BMP signaling). In some embodiments, modulating BMP signaling includes inhibiting, blocking, interfering, or attenuating BMP signaling using one or more BMP inhibitory agents. Non-limiting examples of agents that inhibit BMP activity include: peptide inhibitors, small molecule antagonists, antibodies (or antigen-binding fragments thereof), and/or agents which do not directly bind BMP or BMP signaling components but nonetheless interfere with, block or attenuate BMP-mediated signaling.

In some embodiments, the method includes modulating BMP-mediated signaling by inhibiting BMP signaling. In some embodiments, inhibiting BMP-mediated signaling includes inhibiting signaling associated with BMP type I receptor (e.g., ACVR1, BMPR1A, and BMPR1B). In some embodiments, inhibiting BMP-mediated signaling includes inhibiting signaling associated with an activin receptor like kinase 2 (ALK2) and an activin receptor like kinase 3 (ALK3).

In some embodiments, the method for improving myogenic differentiation capacity of a cell line or an immortalized cell line includes contacting the cell line or the immortalized cell line with a culture media comprising at least a first BMP inhibitor. In some embodiments, the method for improving myogenic differentiation capacity of a cell line or an immortalized cell line includes contacting the cell line or the immortalized cell line with a culture media comprising the first BMP inhibitor and a second BMP inhibitor (e.g., any of the BMP inhibitors described herein).

In some embodiments, BMP-mediated signaling is inhibitor is selected from LDN193189, Dorsomorphin, Noggin, Chrodin, and Gremlin.

In some embodiments, the method includes modulating BMP-mediated signaling by contacting the cell line or the immortalized cell line with a culture media comprising LDN193189 (Cas Number: 1062368-24-4). In some embodiments, the method includes contacting the cell line or the immortalized cell line with a culture media comprising an inhibitor of BMP-mediated signaling (e.g., LDN193189) at a concentration ranging from about 0.2 μM to about 1.0 μM (e.g., about 0.2 μM to about 0.9 about 0.2 μM to about 0.8 about 0.2 μM to about 0.7 about 0.2 μM to about 0.6 about 0.2 μM to about 0.5 about 0.2 μM to about 0.4 about 0.2 μM to about 0.3 about 0.3 μM to about 1.0 about 0.3 to about 0.9 about 0.3 μM to about 0.8 about 0.3 μM to about 0.7 about 0.3 μM to about 0.6 about 0.3 μM to about 0.5 about 0.3 μM to about 0.4 about 0.4 μM to about 1.0 about 0.4 to about 0.9 about 0.4 μM to about 0.8 about 0.4 μM to about 0.7 about 0.4 μM to about 0.6 about 0.4 μM to about 0.5 about 0.5 μM to about 1.0 about 0.5 to about 0.9 about 0.5 μM to about 0.8 about 0.5 μM to about 0.7 about 0.5 μM to about 0.6 about 0.6 μM to about 1.0 about 0.6 to about 0.9 about 0.6 μM to about 0.8 about 0.6 μM to about 0.7 about 0.7 μM to about 1.0 about 0.7 to about 0.9 about 0.7 μM to about 0.8 about 0.8 μM to about 1.0 about 0.8 μM to about 0.9 or about 0.9 μM to about 1.0 In some embodiments, the method includes contacting the cell line or the immortalized cell line with a culture media comprising an inhibitor of BMP-mediated signaling (e.g., LDN193189) at a concentration of about 0.4 μM.

In some embodiments, the method includes modulating BMP-mediated signaling by contacting the cell line or the immortalized cell line with a culture media comprising dorsomorphin (Cas Number: 866405-64-3). In some embodiments, the method includes contacting the cell line or the immortalized cell line with a culture media comprising an inhibitor of BMP-mediated signaling (e.g., dorsomorphin) at a concentration ranging from about 0.1 μM to about 10 μM (or any of the values or subranges therein). In some embodiments, the methods provided herein include contacting the cell line or the immortalized cell line with a culture media comprising dorsomorphin at a concentration of about 1 μM.

In some embodiments, the method includes modulating BMP-mediated signaling using an agent that binds to one or more bone morphogenic proteins (e.g., BMP2 and/or BMP4). Non-limiting examples of agents that bind to and inhibit BMPs proteins include Noggin, Chrodin, and Gremlin. In some embodiments, modulating BMP-mediating signaling includes noggin-mediated antagonism of BMP signaling. By binding to BMPs, Noggin prevents BMPs from binding their receptors, thereby inhibiting BMP-mediated signaling. In some embodiments, the method includes contacting the cell line or the immortalized cell line with a culture media comprising Noggin at a concentration of about 10 ng/mL, about 15 ng/mL, about 20 ng/mL, about 25 ng/mL, about 30 ng/mL, about 35 ng/mL, about 40 ng/mL, about 45 ng/mL, 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, or about 200 ng/mL, about 300 ng/mL, about 400 ng/mL, about 500 ng/mL, about 600 ng/mL, about 700 ng/mL, about 800 ng/mL, about 900 ng/mL, or about 1000 ng/mL.

In some embodiments, modulating BMP signaling includes activating, stabilizing, or inducing BMP signaling using one or more BMP activation agents. Non-limiting examples of agents that activate BMP signaling include: BMP2, BMP4, BMP7, BMP13, and BMP14. In some embodiments, the method includes contacting the cell line or the immortalized cell line with a culture media comprising BMP4 at a concentration of about 1 ng/mL, about 2 ng/mL, about 3 ng/mL, about 4 ng/mL, about 5, ng/mL about 6 ng/mL, about 7 ng/mL, about 8 ng/mL, about 9 ng/mL, about 10 ng/mL, about 11 ng/mL, about 12 ng/mL, about 13 ng/mL, about 14, ng/mL, about 15 ng/mL, about 16 ng/mL, about 17 ng/mL, about 18 ng/mL, about 19 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, or about 100 ng/mL. In some embodiments, the method includes contacting the cell line or the immortalized cell line with a culture media comprising BMP4 at a concentration of about 10 ng/mL.

### 6.2.1.3 WNT

In some embodiments, the method for improving myogenic differentiation capacity of a cell line (e.g., a late passage cell line) or an immortalized cell line includes modulating WNT-mediated signaling (WNT signaling). In some embodiments, modulating WNT signaling includes activating, stabilizing, or inducing WNT signaling using one or more WNT activation agents. Non-limiting examples of agents that activate WNT signaling include: peptides (e.g., growth factors) and, small molecule agonists and/or agents which do not directly bind WNT signaling components but nonetheless activate, stabilize, or induce WNT-mediated signaling.

In some embodiments, the method includes modulating WNT-mediated signaling by inhibiting glycogen synthase kinase 3 (GSK-3). In such cases, inhibiting GSK-3 activates WNT-mediated signaling. GSK3 is a serine/threonine kinase that plays a central role in the regulation of the WNT/β-catenin signaling pathway. Without wishing to be bound by theory, when the WNT ligand is present, it binds its receptor Fzd and the coreceptor lipoprotein-related protein 5 and 6 (LRP-5/6) on the target cell, which signals through disheveled (Dvl) to suppress β-catenin phosphorylation. β-catenin is able to complex with T-cell factor/lymphoid enhancer-binding factor (TCF/LEF) and induce target gene transcription. In the resting state, GSK3 and casein kinase I (CKI) phosphorylate β-catenin, triggering its destabilization and degradation to maintain a low level of β-catenin in the cytosol/nucleus. In such cases, pharmacologic inhibition of GSK3 activity can lead to stabilization and activation of β-catenin and TCF/LEF-dependent gene transcription, which reflects the activity of WNT signal transduction.

In some embodiments, the method for improving myogenic differentiation capacity of a cell line or an immortalized cell line includes contacting the cell line or the immortalized cell line with a culture media comprising at least a first WNT activator. In some embodiments, the method for improving myogenic differentiation capacity of a cell line or an immortalized cell line includes contacting the cell line or the immortalized cell line with a culture media comprising the first WNT activator and a second WNT activator (e.g., any of the WNT activators described herein).

In some embodiments, the WNT-mediated signaling activator is selected from: CHIR99021, BIO, AZD1080, WNT1a, WNT3a, WNT4, and WNT7.

In some embodiments, the GSK-3 inhibitor is CHIR99021. In some embodiments, the method includes contacting the cell line or the immortalized cell line with a culture media comprising a GSK-3 inhibitor (e.g., CHIR99021) at a concentration ranging from about 2.5 μM to about 10 μM (e.g., about 2.5 μM to about 9 μM, about 2.5 μM to about 8 about 2.5 μM to about 7 about 2.5 μM to about 10 about 2.5 μM to about 6 about 2.5 μM to about 5 about 2.5 μM to about 4 μM, 2.5 μM to about 3 about 3 μM to about 10 about 3 μM to about 9 about 3 μM to about 8 about 3 μM to about 7 about 3 μM to about 6 about 3 μM to about 5 μM, about 3 μM to about 4 about 4 μM to about 10 about 4 μM to about 9 about 4 μM to about 8 about 4 μM to about 7 about 4 μM to about 6 about 4 μM to about 5 about 5 μM to about 10 about 5 μM to about 9 about 5 μM to about 8 about 5 μM to about 7 about 5 μM to about 6 about 6 μM to about 10 about 6 μM to about 9 about 6 μM to about 8 about 6 μM to about 7 about 7 μM to about 10 about 7 μM to about 9 about 7 μM to about 8 about 8 μM to about 10 about 8 μM to about 9 or about 9 μM to about 10 In some embodiments, the method includes contacting the cell line or the immortalized cell line with a culture media comprising a GSK-3 inhibitor (e.g., CHIR99021) at a concentration of about 5 μM.

Other GSK-3 inhibitors that can be used in the methods described herein include, without limitation: LY2090314 (Cas Number: 603288-22-8), BIO (Cas Number: 667463-62-9), and AZD1080 (Cas Number: 612487-72-6).

In some embodiments, the method includes modulating WNT mediated signaling using an agent that is a WNT signaling agonist. Non-limiting examples of WNT signaling agonists include WNT1a, WNT3a, WNT4, and WNT7. In some embodiments, the WNT signaling agonist is WNT1a. In some embodiments, the methods provided herein include exposing the immortalized cell line to WNT1a at a concentration of about 2.5 ng/mL, about 5 ng/mL, about 7.5 ng/mL about 10 ng/mL, about 12.5 ng/mL, about 15 ng/mL, about 17.5 ng/mL, about 20 ng/mL, about 22.5 ng/mL, about 25 ng/mL, about 27.5 ng/mL, about 30 ng/mL, about 32.5 ng/mL, about 35 ng/mL, about 37.5 ng/mL, about 40 ng/mL, about 42.5 ng/mL, about 45 ng/mL, about 47.5 ng/mL, about 50 ng/mL, about 55 ng/mL about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, or about 200 ng/mL, about 300 ng/mL, about 400 ng/mL, about 500 ng/mL, about 600 ng/mL, about 700 ng/mL, about 800 ng/mL, about 900 ng/mL, or about 1000 ng/mL.

In some embodiments, modulating WNT signaling includes inhibiting, blocking, or interfering with WNT signaling using one or more WNT inhibitory agents. Non-limiting examples of agents that inhibit WNT signaling include: peptide inhibitors, small molecule antagonists, antibodies (or antigen-binding fragments thereof), and/or agents which do not directly bind WNT signaling components but nonetheless interfere with, block or attenuate WNT-mediated signaling.

In some embodiments, the WNT signaling inhibitor is IWR1 (Cas Number 1127442-82-3). In some embodiments, the method includes contacting the cell line or the immortalized cell line with a culture media comprising IRW1 at a concentration ranging from about 0.25 μM to about 5 μM (e.g., about 0.25 μM to about 4.75 μM, about 0.25 μM to about 4.5 μM, about 0.25 μM to about 4.25 μM, about 0.25 μM to about 4 μM, about 0.25 μM to about 3.75 μM, about 0.25 μM to about 3.5 μM, about 0.25 μM to about 3.25 μM, about 0.25 μM to about 3.0 μM, about 0.25 μM to about 2.75 μM, about 0.25 μM to about 2.5 μM, about 0.25 μM to about 2.25 μM, about 0.25 μM to about 2.0 μM, about 0.25 μM to about 1.75 μM, about 0.25 μM to about 1.5 μM, about 0.25 μM to about 1.25 μM, about 0.25 μM to about 1.0 μM, about 0.25 μM to about 0.75 μM, about 0.25 μM to about 0.5 μM, about 0.5 μM to about 5 μM, about 0.5 μM to about 4.75 μM, about 0.5 μM to about 4.5 μM, about 0.5 μM to about 4.25 μM, about 0.5 μM to about 4 μM, about 0.5 μM to about 3.75 μM, about 0.5 μM to about 3.5 μM, about 0.5 μM to about 3.25 μM, about 0.5 μM to about 3.0 μM, about 0.5 μM to about 2.75 μM, about 0.5 μM to about 2.5 μM, about 0.5 μM to about 2.25 μM, about 0.5 μM to about 2.0 μM, about 0.5 μM to about 1.75 μM, about 0.5 μM to about 1.5 μM, about 0.5 μM to about 1.25 μM, about 0.5 μM to about 1.0 μM, about 0.5 μM to about 0.75 μM, about 0.75 μM to about 5 μM, about 0.75 μM to about 4.75 μM, about 0.75 μM to about 4.5 μM, about 0.75 μM to about 4.25 μM, about 0.75 μM to about 4 μM, about 0.75 μM to about 3.75 μM, about 0.75 μM to about 3.5 μM, about 0.75 μM to about 3.25 μM, about 0.75 μM to about 3.0 μM, about 0.75 μM to about 2.75 μM, about 0.75 μM to about 2.5 μM, about 0.75 μM to about 2.25 μM, about 0.75 μM to about 2.0 μM, about 0.75 μM to about 1.75 μM, about 0.75 μM to about 1.5 μM, about 0.75 μM to about 1.25 μM, about 0.75 μM to about 1.0 μM, about 1.0 μM to about 5 μM, about 1.0 μM to about 4.75 μM, about 1.0 μM to about 4.5 μM, about 1.0 μM to about 4.25 μM, about 1.0 μM to about 4 μM, about 1.0 μM to about 3.75 μM, about 1.0 μM to about 3.5 μM, about 1.0 μM to about 3.25 μM, about 1.0 μM to about 3.0 μM, about 1.0 μM to about 2.75 μM, about 1.0 μM to about 2.5 μM, about 1.0 μM to about 2.25 μM, about 1.0 μM to about 2.0 μM, about 1.0 μM to about 1.75 μM, about 1.0 μM to about 1.5 μM, about 1.0 μM to about 1.25 μM, about 1.25 μM to about 5 μM, about 1.25 μM to about 4.75 μM, about 1.25 μM to about 4.5 μM, about 1.25 μM to about 4.25 μM, about 1.25 μM to about 4 μM, about 1.25 μM to about 3.75 μM, about 1.25 μM to about 3.5 μM, about 1.25 μM to about 3.25 μM, about 1.25 μM to about 3.0 μM, about 1.25 μM to about 2.75 μM, about 1.25 μM to about 2.5 μM, about 1.25 μM to about 2.25 μM, about 1.25 μM to about 2.0 μM, about 1.25 μM to about 1.75 μM, about 1.25 μM to about 1.5 μM, about 1.5 μM to about 5 μM, about 1.5 μM to about 4.75 μM, about 1.5 μM to about 4.5 μM, about 1.5 μM to about 4.25 μM, about 1.5 μM to about 4 μM, about 1.5 μM to about 3.75 μM, about 1.5 μM to about 3.5 μM, about 1.5 μM to about 3.25 μM, about 1.5 μM to about 3.0 μM, about 1.5 μM to about 2.75 μM, about 1.5 μM to about 2.5 μM, about 1.5 μM to about 2.25 μM, about 1.5 μM to about 2.0 μM, about 1.5 μM to about 1.75 μM, about 1.75 μM to about 5 μM, about 1.75 μM to about 4.75 μM, about 1.75 μM to about 4.5 μM, about 1.75 μM to about 4.25 μM, about 1.75 μM to about 4 μM, about 1.75 μM to about 3.75 μM, about 1.75 μM to about 3.5 μM, about 1.75 μM to about 3.25 μM, about 1.75 μM to about 3.0 μM, about 1.75 μM to about 2.75 μM, about 1.75 μM to about 2.5 μM, about 1.75 μM to about 2.25 μM, about 1.75 μM to about 2.0 μM, about 2.0 μM to about 5 μM, about 2.0 μM to about 4.75 μM, about 2.0 μM to about 4.5 μM, about 2.0 μM to about 4.25 μM, about 2.0 μM to about 4 μM, about 2.0 μM to about 3.75 μM, about 2.0 μM to about 3.5 μM, about 2.0 μM to about 3.25 μM, about 2.0 μM to about 3.0 μM, about 2.0 μM to about 2.75 μM, about 2.0 μM to about 2.5 μM, about 2.0 μM to about 2.25 μM, about 2.25 μM to about 5 μM, about 2.25 μM to about 4.75 μM, about 2.25 μM to about 4.5 μM, about 2.25 μM to about 4.25 μM, about 2.25 μM to about 4 μM, about 2.25 μM to about 3.75 μM, about 2.25 μM to about 3.5 μM, about 2.25 μM to about 3.25 μM, about 2.25 μM to about 3.0 μM, about 2.25 μM to about 2.75 μM, about 2.25 μM to about 2.5 μM, about 2.5 μM to about 5 μM, about 2.5 μM to about 4.75 μM, about 2.5 μM to about 4.5 μM, about 2.5 μM to about 4.25 μM, about 2.5 μM to about 4 μM, about 2.5 μM to about 3.75 μM, about 2.5 μM to about 3.5 μM, about 2.5 μM to about 3.25 μM, about 2.5 μM to about 3.0 μM, about 2.5 μM to about 2.75 μM, about 2.75 μM to about 5 μM, about 2.75 μM to about 4.75 μM, about 2.75 μM to about 4.5 μM, about 2.75 μM to about 4.25 μM, about 2.75 μM to about 4 μM, about 2.75 μM to about 3.75 μM, about 2.75 μM to about 3.5 μM, about 2.75 μM to about 3.25 μM, about 2.75 μM to about 3.0 μM, about 3.0 μM to about 5 μM, about 3.0 μM to about 4.75 μM, about 3.0 μM to about 4.5 μM, about 3.0 μM to about 4.25 μM, about 3.0 μM to about 4 μM, about 3.0 μM to about 3.75 μM, about 3.0 μM to about 3.5 μM, about 3.0 μM to about 3.25 μM, about 3.25 μM to about 5 μM, about 3.25 μM to about 4.75 μM, about 3.25 μM to about 4.5 μM, about 3.25 μM to about 4.25 μM, about 3.25 μM to about 3.75 μM, about 3.25 μM to about 3.5 μM, about 3.25 μM to about 4 μM, about 3.5 μM to about 5 μM, about 3.5 μM to about 4.75 μM, about 3.5 μM to about 4.5 μM, about 3.5 μM to about 4.25 μM, about 3.5 μM to about 4 μM, about 3.5 μM to about 3.75 μM, about 3.75 μM to about 5 μM, about 3.75 μM to about 4.75 μM, about 3.75 μM to about 4.5 μM, about 3.75 μM to about 4.25 μM, about 3.75 μM to about 4 μM, about 4.0 μM to about 5 μM, about 4.0 μM to about 4.75 μM, about 4.0 μM to about 4.5 μM, about 4.0 μM to about 4.25 μM, about 4.25 μM to about 5 μM, about 4.25 μM to about 4.75 μM, about 4.25 μM to about 4.5 μM, about 4.5 μM to about 5 μM, about 4.5 μM to about 4.75 μM, or about 4.75 μM to about 5 μM). In some embodiments, the method includes contacting the cell line or the immortalized cell line with a culture media comprising IRW1 at a concentration of about 2.5 μM.

### 6.2.1.4 Epigenetic Modulators

In some embodiments, the method for improving myogenic differentiation capacity of a cell line (e.g., a late passage cell line) or an immortalized cell line includes contacting the cell line or the immortalized cell line with a culture media comprising an epigenetic modulator. Non-limiting examples of epigenetic modulators include: sodium butyrate, 5-Aza-Cytidine, RG108, scriptaid, trichostatin A, suberoylanilide hydroxamic Acid, MS-275, CI-994, BML-210, M344, MGCD0103, PXD101, LBH-589, tubastatin A, NSC3825, NCH-51, NSC-3852, HNHA, BML-281, CBHA, salermide, pimelic diphenylamide, ITF-2357, PCI-24781, APHA Compound 8, Droxinostat, and SB-939, histone deacetylase paralogs, histone acetyltransferase paralogs, tet-methylcytosine dioxygenase paralogs, histone demethylase paralogs, histone methyltransferase paralogs, and DNA methyltransferase paralogs, histones, and subunits of chromatin remodeling complexes including Mi-2/NuRD and SWI/SNF.

In some embodiments, the method for improving myogenic differentiation capacity of a cell line (e.g., a late passage cell line) or an immortalized cell line includes contacting the cell line or the immortalized cell line with a culture media comprising an agent that inhibits histone deacetylase (HDAC) activity. In some embodiments, the HDAC inhibitor is sodium butyrate (Cas Number 156-54-7). In some embodiments, exposing the cells (e.g., the immortalized cell line) to sodium butyrate results in histone hyperacetylation. In some embodiments, sodium butyrate inhibits class I histone deacetylase (HDAC) activity, including HDAC1, HDAC2, HDAC3.

In some embodiments, the method includes contacting the cell line or the immortalized cell line with a culture media comprising an HDAC inhibitor (e.g., sodium butyrate) at a concentration ranging from about 0.05 mM to about 5 mM (e.g., about 0.05 mM to about 4.75 mM, about 0.05 mM to about 4.5 mM, about 0.05 mM to about 4.25 mM, about 0.05 mM to about 4.0 mM, about 0.05 mM to about 3.75 mM, about 0.05 mM to about 3.5 mM, about 0.05 mM to about 3.25 mM, about 0.05 mM to about 3.0 mM, about 0.05 mM to about 2.75 mM, about 0.05 mM to about 2.5 mM, about 0.05 mM to about 2.25 mM, about 0.05 mM to about 2.0 mM, about 0.05 mM to about 1.75 mM, about 0.05 mM to about 1.5 mM, about 0.05 mM to about 1.25 mM, about 0.05 mM to about 1.0 mM, about 0.05 mM to about 0.75 mM, about 0.05 mM to about 0.5 mM, about 0.05 mM to about 0.25 mM, about 0.05 mM to about 0.1 mM, about 0.1 mM to about 5 mM, about 0.1 mM to about 4.75 mM, about 0.1 mM to about 4.5 mM, about 0.1 mM to about 4.25 mM, about 0.1 mM to about 4.0 mM, about 0.1 mM to about 3.75 mM, about 0.1 mM to about 3.5 mM, about 0.1 mM to about 3.25 mM, about 0.1 mM to about 3.0 mM, about 0.1 mM to about 2.75 mM, about 0.1 mM to about 2.5 mM, about 0.1 mM to about 2.25 mM, about 0.1 mM to about 2.0 mM, about 0.1 mM to about 1.75 mM, about 0.1 mM to about 1.5 mM, about 0.1 mM to about 1.25 mM, about 0.1 mM to about 1.0 mM, about 0.1 mM to about 0.75 mM, about 0.1 mM to about 0.5 mM, about 0.1 mM to about 0.25 mM, about 0.25 mM to about 5 mM, about 0.25 mM to about 4.75 mM, about 0.25 mM to about 4.5 mM, about 0.25 mM to about 4.25 mM, about 0.25 mM to about 4.0 mM, about 0.25 mM to about 3.75 mM, about 0.25 mM to about 3.5 mM, about 0.25 mM to about 3.25 mM, about 0.25 mM to about 3.0 mM, about 0.25 mM to about 2.75 mM, about 0.25 mM to about 2.5 mM, about 0.25 mM to about 2.25 mM, about 0.25 mM to about 2.0 mM, about 0.25 mM to about 1.75 mM, about 0.25 mM to about 1.5 mM, about 0.25 mM to about 1.25 mM, about 0.25 mM to about 1.0 mM, about 0.25 mM to about 0.75 mM, about 0.25 mM to about 0.5 mM, about 0.5 mM to about 5 mM, about 0.5 mM to about 4.75 mM, about 0.5 mM to about 4.5 mM, about 0.5 mM to about 4.25 mM, about 0.5 mM to about 4.0 mM, about 0.5 mM to about 3.75 mM, about 0.5 mM to about 3.5 mM, about 0.5 mM to about 3.25 mM, about 0.5 mM to about 3.0 mM, about 0.5 mM to about 2.75 mM, about 0.5 mM to about 2.5 mM, about 0.5 mM to about 2.25 mM, about 0.5 mM to about 2.0 mM, about 0.5 mM to about 1.75 mM, about 0.5 mM to about 1.5 mM, about 0.5 mM to about 1.25 mM, about 0.5 mM to about 1.0 mM, about 0.5 mM to about 0.75 mM, about 0.75 mM to about 5 mM, about 0.75 mM to about 4.75 mM, about 0.75 mM to about 4.5 mM, about 0.75 mM to about 4.25 mM, about 0.75 mM to about 4.0 mM, about 0.75 mM to about 3.75 mM, about 0.75 mM to about 3.5 mM, about 0.75 mM to about 3.25 mM, about 0.75 mM to about 3.0 mM, about 0.75 mM to about 2.75 mM, about 0.75 mM to about 2.5 mM, about 0.75 mM to about 2.25 mM, about 0.75 mM to about 2.0 mM, about 0.75 mM to about 1.75 mM, about 0.75 mM to about 1.5 mM, about 0.75 mM to about 1.25 mM, about 0.75 mM to about 1.0 mM, about 1.0 mM to about 5 mM, about 1.0 mM to about 4.75 mM, about 1.0 mM to about 4.5 mM, about 1.0 mM to about 4.25 mM, about 1.0 mM to about 4.0 mM, about 1.0 mM to about 3.75 mM, about 1.0 mM to about 3.5 mM, about 1.0 mM to about 3.25 mM, about 1.0 mM to about 3.0 mM, about 1.0 mM to about 2.75 mM, about 1.0 mM to about 2.5 mM, about 1.0 mM to about 2.25 mM, about 1.0 mM to about 2.0 mM, about 1.0 mM to about 1.75 mM, about 1.0 mM to about 1.5 mM, about 1.0 mM to about 1.25 mM, about 1.25 mM to about 5 mM, about 1.25 mM to about 4.75 mM, about 1.25 mM to about 4.5 mM, about 1.25 mM to about 4.25 mM, about 1.25 mM to about 4.0 mM, about 1.25 mM to about 3.75 mM, about 1.25 mM to about 3.5 mM, about 1.25 mM to about 3.25 mM, about 1.25 mM to about 3.0 mM, about 1.25 mM to about 2.75 mM, about 1.25 mM to about 2.5 mM, about 1.25 mM to about 2.25 mM, about 1.25 mM to about 2.0 mM, about 1.25 mM to about 1.75 mM, about 1.25 mM to about 1.5 mM, about 1.5 mM to about 5 mM, about 1.5 mM to about 4.75 mM, about 1.5 mM to about 4.5 mM, about 1.5 mM to about 4.25 mM, about 1.5 mM to about 4.0 mM, about 1.5 mM to about 3.75 mM, about 1.5 mM to about 3.5 mM, about 1.5 mM to about 3.25 mM, about 1.5 mM to about 3.0 mM, about 1.5 mM to about 2.75 mM, about 1.5 mM to about 2.5 mM, about 1.5 mM to about 2.25 mM, about 1.5 mM to about 2.0 mM, about 1.5 mM to about 1.75 mM, about 1.75 mM to about 5 mM, about 1.75 mM to about 4.75 mM, about 1.75 mM to about 4.5 mM, about 1.75 mM to about 4.25 mM, about 1.75 mM to about 4.0 mM, about 1.75 mM to about 3.75 mM, about 1.75 mM to about 3.5 mM, about 1.75 mM to about 3.25 mM, about 1.75 mM to about 3.0 mM, about 1.75 mM to about 2.75 mM, about 1.75 mM to about 2.5 mM, about 1.75 mM to about 2.25 mM, about 1.75 mM to about 2.0 mM, about 2.0 mM to about 5 mM, about 2.0 mM to about 4.75 mM, about 2.0 mM to about 4.5 mM, about 2.0 mM to about 4.25 mM, about 2.0 mM to about 4.0 mM, about 2.0 mM to about 3.75 mM, about 2.0 mM to about 3.5 mM, about 2.0 mM to about 3.25 mM, about 2.0 mM to about 3.0 mM, about 2.0 mM to about 2.75 mM, about 2.0 mM to about 2.5 mM, about 2.0 mM to about 2.25 mM, about 2.25 mM to about 5 mM, about 2.25 mM to about 4.75 mM, about 2.25 mM to about 4.5 mM, about 2.25 mM to about 4.25 mM, about 2.25 mM to about 4.0 mM, about 2.25 mM to about 3.75 mM, about 2.25 mM to about 3.5 mM, about 2.25 mM to about 3.25 mM, about 2.25 mM to about 3.0 mM, about 2.25 mM to about 2.75 mM, about 2.25 mM to about 2.5 mM, about 2.5 mM to about 5 mM, about 2.5 mM to about 4.75 mM, about 2.5 mM to about 4.5 mM, about 2.5 mM to about 4.25 mM, about 2.5 mM to about 4.0 mM, about 2.5 mM to about 3.75 mM, about 2.5 mM to about 3.5 mM, about 2.5 mM to about 3.25 mM, about 2.5 mM to about 3.0 mM, about 2.5 mM to about 2.75 mM, about 2.75 mM to about 5 mM, about 2.75 mM to about 4.75 mM, about 2.75 mM to about 4.5 mM, about 2.75 mM to about 4.25 mM, about 2.75 mM to about 4.0 mM, about 2.75 mM to about 3.75 mM, about 2.75 mM to about 3.5 mM, about 2.75 mM to about 3.25 mM, about 2.75 mM to about 3.0 mM, about 3.0 mM to about 5 mM, about 3.0 mM to about 4.75 mM, about 3.0 mM to about 4.5 mM, about 3.0 mM to about 4.25 mM, about 3.0 mM to about 4.0 mM, about 3.0 mM to about 3.75 mM, about 3.0 mM to about 3.5 mM, about 3.0 mM to about 3.25 mM, about 3.25 mM to about 5 mM, about 3.25 mM to about 4.75 mM, about 3.25 mM to about 4.5 mM, about 3.25 mM to about 4.25 mM, about 3.25 mM to about 4.0 mM, about 3.25 mM to about 3.75 mM, about 3.25 mM to about 3.5 mM, about 3.5 mM to about 5 mM, about 3.5 mM to about 4.75 mM, about 3.5 mM to about 4.25 mM, about 3.5 mM to about 4.5 mM, about 3.5 mM to about 4.25 mM, about 3.5 mM to about 4.0 mM, about 3.5 mM to about 3.75 mM, about 3.75 mM to about 5 mM, about 3.75 mM to about 4.75 mM, about 3.75 mM to about 4.5 mM, about 3.75 mM to about 4.25 mM, about 3.75 mM to about 4.0 mM, about 4.25 mM to about 5 mM, about 4.25 mM to about 4.75 mM, about 4.25 mM to about 4.5 mM, about 4.25 mM to about 4.25 mM, about 4.5 mM to about 5 mM, about 4.5 mM to about 4.75 mM, or about 4.75 mM to about 5). In some embodiments, the method includes contacting the cell line or the immortalized cell line with a culture media comprising an HDAC inhibitor (e.g., sodium butyrate) at a concentration of about 0.5 mM. In some embodiments, the method includes contacting the cell line or the immortalized cell line with a culture media comprising an HDAC inhibitor (e.g., sodium butyrate) at a concentration of about 1.0 mM.

In some embodiments, the method includes contacting a cell line or an immortalized cell line engineered to express MYOD to a culture media (e.g., see FIG. 2, ME9) comprising the HDAC inhibitor (e.g., sodium butyrate). In some embodiments, the method includes contacting a cell line or an immortalized cell line engineered to express PAX7, MYOD, and MEF2B (or one or more of any of the other myogenic regulatory factors (e.g., 7MM)) with a culture media (e.g., see FIG. 2, ME9) including an Activin A inhibitor, a BMP inhibitor, a WNT activator, or a combination thereof, and the HDAC inhibitor (e.g., sodium butyrate).

In some embodiments, the method includes contacting a cell line or an immortalized cell line with a culture media comprising a Activin A inhibitor, a BMP inhibitor, optionally, a WNT activator, and the HDAC inhibitor (e.g., sodium butyrate).

In some embodiments, contacting the cell line or the immortalized cell line with a culture media comprising an HDAC inhibitor (e.g., sodium butyrate) occurs for a period of time under conditions that allow for improvements in differentiation capacity. In some embodiments, contacting the cell line or the immortalized cell line with a culture media comprising an HDAC inhibitor (e.g., sodium butyrate) occurs for a period of time of about 1 day, about 2 days, about 3 days, about 4 days, about 5 days, about 6 days, about 7 days, about 8 days, about 9 days, about 10 days, about 11 days about 12 days, about 13 days or about 14 days. In some embodiments, exposing the immortalized cell line to an HDAC inhibitor (e.g., sodium butyrate) occurs for a period of time of about one week, about two weeks, about three weeks, about four weeks, about five weeks, about six weeks, about seven weeks or about eight weeks.

In some embodiments, the steps of contacting the cell line or the immortalized cell line with a culture media comprising at least a first Activin A inhibitor and at least a first BMP inhibitor (and optionally a WNT activator) and exposing the cell line or the immortalized cell to an HDAC inhibitor (e.g., sodium butyrate) are performed sequentially. For example, the cell line or the immortalized cell line is first exposed to an at least a first Activin A inhibitor and at least a first BMP inhibitor (and optionally a WNT activator) prior to being exposed to the HDAC inhibitor. In some embodiments, the steps of contacting the immortalized cell line to an at least a first Activin A inhibitor and at least a first BMP inhibitor (and optionally a WNT activator) and exposing the immortalized cell to an HDAC inhibitor (e.g., sodium butyrate) are performed with a rest period (e.g., a rest period of about 3 hours to about 3 days) in between exposures. In some embodiments, the steps of contacting the immortalized cell line with a culture media comprising an at least a first Activin A inhibitor and at least a first BMP inhibitor (and optionally a WNT activator) and exposing the immortalized cell to an HDAC inhibitor (e.g., sodium butyrate) are performed with no rest period in between exposures.

In some embodiments, as a result of contacting the cell line or the immortalized cell line with a culture media comprising an HDAC inhibitor (e.g., sodium butyrate), the myogenic differentiation capacity of the cell line or the immortalized cell line is increased as compared to a cell line or an immortalized cell line not contacted with an HDAC inhibitor. In some embodiments, as a result of contacting the cell line or the immortalized cell line with a culture media comprising an HDAC inhibitor (e.g., sodium butyrate), the cell line or the immortalized cell experiences an increase in Pax7 expression, an increase MyHC1 expression, and/or an increase the ability to form myotubes as compared to a cell line or an immortalized cell line not contacted with an HDAC inhibitor

### 6.2.2. Myogenic Regulatory Factors for Improving Differentiation Capacity

In some embodiments, the methods provided herein include introducing into, or incorporating into the genome of, a cell (e.g., a cell of an immortalized cell line) a polynucleotide encoding at least a first myogenic regulatory factor polypeptide. Transforming (e.g., introducing or incorporating into the genome of) a cell (e.g., a cell of an immortalized cell line) with one or more myogenic regulatory factor polypeptides improves the differentiation capacity of the immortalized cell line. In such cases, the cell line (e.g., the immortalized cell line) is better suited to produce cell types of interest, for example, cell types used for cultured food production (e.g., myoblasts).

In some embodiments, a cell (e.g., a cell of an immortalized cell line) is transformed with two or more, three or more, four or more, or five or more myogenic regulatory factors. In some embodiments, two or more, three or more, four or more, or five or more myogenic regulatory factors are introduced into or incorporated into the genome of a cell (e.g., a cell of an immortalized cell line). In some embodiments, each additional myogenic regulatory factor transformed, introduced, or incorporated into the cell line (e.g., the immortalized cell line) further improves the differentiation capacity of the cell line (e.g., the immortalized cell line). In cases where two or more myogenic regulatory factors are transformed, introduced, or incorporated into a cell, the two or more myogenic regulatory factors are present in one polynucleotide sequence. Alternatively, in cases where two or more myogenic regulatory factors are transformed, introduced, or incorporated into a cell, each of the two or more myogenic regulatory factors are on different polynucleotide sequences.

In some embodiments, the one or more myogenic regulatory factors are selected from: MYOD, MYOG, PAX7, PAX3, MEF2B, and PITX1. In some embodiments, transforming, introducing, or incorporating one or more nucleic acid sequences encoding PAX3/7 or a fragment thereof, MEF2B or a fragment thereof, and PITX1 or a fragment thereof, into a cell (e.g., a cell of an immortalized cell line) improves differentiation capacity of the cell (e.g., the cell of the immortalized cell line). In some embodiments, transforming one or more nucleic acid sequences encoding MYOD or a fragment thereof, PAX7 or a fragment thereof, and MEF2B or a fragment thereof, into a cell (e.g., a cell of an immortalized cell line) improves differentiation capacity of the cell (e.g., the cell of the immortalized cell line). In some embodiments, transforming one or more nucleic acid sequences encoding MYOD or a fragment thereof, PAX7 or a fragment thereof, MEF2B or a fragment thereof, and PITX1 or a fragment thereof, into a cell (e.g., a cell of an immortalized cell line) improves differentiation capacity of the cell (e.g., the cell of the immortalized cell line).

In some embodiments, the nucleic acid sequence encoding the one or more myogenic regulatory factors can be from any organism. In some embodiments, the nucleic acid sequence encoding the one or more myogenic regulatory factors can be from any animal, such as vertebrate and invertebrate animal species.

Non-limiting examples and descriptions of each myogenic regulatory factor is described below.

### 6.2.2.1 MYOD

In some embodiments, a cell line or an immortalized cell line is transformed with a nucleic acid sequence encoding a MYOD polypeptide, or a fragment thereof. As used herein, “MyoD” refers to the myogenic differentiation 1 (MyoD1) gene or MYOD or MYDO1 protein that is a nuclear protein that belongs to the basic helix-loop-helix family of transcription factors and the myogenic factors subfamily. MYOD1 regulates muscle cell differentiation and muscle regeneration. MYOD1 activates its own transcription which may stabilize commitment to myogenesis, and acts as a transcriptional activator that promotes transcription of muscle-specific target genes. In some embodiments, MyoD refers to the MyoD1 gene or MYOD or MYOD1 polypeptide, or a variant thereof (e.g., a MYOD polypeptide having one or more (e.g., one, two, three, four, five, six, seven, eight, nine, ten, or more amino acid substitutions, deletions or insertions as compared to a wild type MYOD polypeptide)). Non-limiting examples of MYOD polypeptides are described in Table 1.

In some embodiments referring to a MYOD polypeptide, the amino acid sequence of the MYOD polypeptide is at least 80% (e.g., at least 85%, 90, 95%, 96%, 97%, 98%, or 99%) identical to SEQ ID NOs: 1-6.

In some embodiments, transforming a nucleic acid sequence encoding MyoD alone is not sufficient to improve differentiation capacity in the immortalized cell line.

### 6.2.2.2 MYOG

In some embodiments, a cell line or an immortalized cell line is transformed with a nucleic acid sequence encoding a MYOG polypeptide, or a fragment thereof. As used herein, “MyoG” refers to the Myogenin (MyoG) or MYOG polypeptide that is a muscle-specific transcription factor. MyoG is a helix-loop-helix (HLH) protein that is essential for development and function of skeletal muscle. In some embodiments, MyoG refers to a MyoG gene or MYOG polypeptide, or a variant thereof (e.g., a MYOG polypeptide having one or more (e.g., one, two, three, four, five, six, seven, eight, nine, ten, or more amino acid substitutions, deletions or insertions as compared to a wild type MYOG polypeptide)). Non-limiting examples of MYOD polypeptides are as described in Table 1.

In some embodiments referring to a MYOG polypeptide, the amino acid sequence of the MYOG polypeptide is at least 80% (e.g., at least 85%, 90, 95%, 96%, 97%, 98%, or 99%) identical to SEQ ID NOs: 7-12.

### 6.2.2.3 PAX7

In some embodiments, a cell line or an immortalized cell line is transformed with a nucleic acid sequence encoding a MEF2B polypeptide, or a fragment thereof. As used herein, “Pax7” refers to paired box 7 (Pax7) gene or PAX7 polypeptide that is a member of the paired box (PAX) family of transcription factors that typically contain a paired box domain, an octapeptide, and a paired-type homeodomain. These genes play critical roles during muscle development. In some embodiments, Pax7 refers to a Pax7 gene or PAX7 polypeptide, or a variant thereof (e.g., a PAX7 polypeptide having one or more (e.g., one, two, three, four, five, six, seven, eight, nine, ten, or more amino acid substitutions, deletions or insertions as compared to a wild type PAX7 polypeptide)). Non-limiting examples of PAX7 polypeptides are as described in Table 1.

In some embodiments, referring to a Pax7 polypeptide, the amino acid sequence of the Pax7 polypeptide is at least 80% (e.g., at least 85%, 90, 95%, 96%, 97%, 98%, or 99%) identical to SEQ ID NOs: 13-18.

### 6.2.2.4 PAX3

In some embodiments, a cell line or an immortalized cell line is transformed with a nucleic acid sequence encoding a PAX3 polypeptide, or a fragment thereof. As used herein, “PAX3” refers to paired box 3 (PAX3) gene or PAX3 polypeptide that is a member of the paired box (PAX) family of transcription factors. Members of the PAX family typically contain a paired box domain and a paired-type homeodomain. These genes play critical roles during fetal development. In some embodiments, Pax3 refers to a Pax3 gene or PAX3 polypeptide, or a variant thereof (e.g., a PAX3 polypeptide having one or more (e.g., one, two, three, four, five, six, seven, eight, nine, ten, or more amino acid substitutions, deletions or insertions as compared to a wild type PAX3 polypeptide)). Non-limiting examples of PAX3 polypeptides are as described in Table 1.

In some embodiments referring to a PAX3 polypeptide, the amino acid sequence of the PAX3 polypeptide is at least 80% (e.g., at least 85%, 90, 95%, 96%, 97%, 98%, or 99%) identical to SEQ ID NOs: 19-24.

### 6.2.2.5 MEF2B

In some embodiments, a cell line or an immortalized cell line is transformed with a nucleic acid sequence encoding a MEF2B polypeptide, or a fragment thereof. As used herein, “Mef2b” refers to myocyte enhancer factor 2B (MEF2B) gene or MEF2B polypeptide that is a member of the MADS/MEF2 family of DNA binding proteins. MEF2B protein regulates gene expression, including expression of the smooth muscle myosin heavy chain gene. In some embodiments, Mef2b refers to a Mef2b gene or MEF2B polypeptide, or a variant thereof (e.g., a MEF2B polypeptide having one or more (e.g., one, two, three, four, five, six, seven, eight, nine, ten, or more amino acid substitutions, deletions or insertions as compared to a wild type MEF2B polypeptide)). Non-limiting examples of MEF2B polypeptides are as described in Table 1.

In some embodiments referring to a MEF2B polypeptide, the amino acid sequence of the MEF2B polypeptide is at least 80% (e.g., at least 85%, 90, 95%, 96%, 97%, 98%, or 99%) identical to SEQ ID NOs: 25-30.

### 6.2.2.6 PITX1

In some embodiments, a cell line or an immortalized cell line is transformed with a nucleic acid sequence encoding a PITX1 polypeptide, or a fragment thereof. As used herein, “PITX1” refers to paired-like homeodomain (PITX1) gene or PITX1 polypeptide that is a member of the RIEG/PITX homeobox family, which is in the bicoid class of homeodomain proteins. Members of this family are involved in organ development and left-right asymmetry. PITX1 acts as a transcriptional regulator. In some embodiments, Pitx1 refers to a Pitx1 gene or PITX1 polypeptide, or a variant thereof (e.g., a PITX1 polypeptide having one or more (e.g., one, two, three, four, five, six, seven, eight, nine, ten, or more amino acid substitutions, deletions or insertions as compared to a wild type PITX1 polypeptide)). Non-limiting examples of PITX1 polypeptides are as described in Table 1.

In some embodiments referring to a PITX1 polypeptide, the amino acid sequence of the PITX1 polypeptide is at least 80% (e.g., at least 85%, 90, 95%, 96%, 97%, 98%, or 99%) identical to SEQ ID NOs: 31-36.

### 6.3. Immortalization

Also provided herein are methods for immortalizing (e.g., extending the renewal capacity) a cell or a cell line. In some embodiments, the cells are modified to express a nucleic acid sequence encoding TERT. As used herein, “TERT” refers to telomerase reverse transcriptase (TERT) gene or TERT polypeptide that is a ribonucleoprotein polymerase that maintains telomere ends by addition of the telomere repeat TTAGGG. Telomerase expression plays a role in cellular senescence, as it is normally repressed in postnatal somatic cells resulting in progressive shortening of telomeres. Non-limiting examples of TERT polypeptides are described in Table 1.

In some embodiments, referring to a TERT polypeptide, the amino acid sequence of the TERT polypeptide is at least 80% (e.g., at least 85%, 90, 95%, 96%, 97%, 98%, or 99%) identical to SEQ ID NOs: 37-42.

In some embodiments, immortalization comprises transforming, introducing, or incorporating into the genome of a cell nucleic acid sequence encoding a telomerase reverse transcriptase (TERT) gene. In some embodiments, cells ectopically express the TERT polynucleotide. In some embodiments, the cells are genetically modified and carry stable integrations of one or more copies of the TERT polynucleotide.

In some embodiments, increased expression of TERT may be achieved using different approaches. In some embodiments, increased expression of TERT may be achieved by ectopically expressing TERT. In some embodiments, increased expression of TERT may be achieved by introducing targeted mutations in the native TERT promoter. In some embodiments, increased expression of TERT may be achieved by activating endogenous TERT expression by an engineered transcriptional activator. In some embodiments, increased expression of TERT may be achieved by transiently transfecting TERT mRNA.

The polynucleotide encoding TERT can be from any organism. The TERT polynucleotide can be from bacteria, plants, fungi, and archaea. The TERT polynucleotide can be from any animal, such as vertebrate and invertebrate animal species. The TERT polynucleotide can be from any vertebrate animal species such as mammals, reptiles, birds, amphibians, and the like. The TERT polynucleotide can be from any mammalian species, such as a human, murine, bovine, porcine, and the like.

In some embodiments, immortalization occurs prior to performing a method for improving differentiation capacity. In some embodiments, a method for improving differentiation capacity further comprises an immortalization step.

Exemplary methods for immortalizing a cell line are as described in WO2019014652A1, which is herein incorporated by reference in its entirety.

TABLE 1
Polypeptides and Nucleic Acid Sequences

| | | NCBI | | |
|---|---|---|---|---|
| Gene | | Protein | NCBI mRNA | |
| Symbol | Species | Ref. No. | Ref. No. | Sequence |
| MyoD | Gallus | NP_989545.3 | NM_204214.3 | MDLLGPMEMTEGSLCSFTAADDFYD |
| | gallus | (SEQ ID | (encodes | DPCFNTSDMHFFEDLDPRLVHVGGLL |
| | (chicken) | NO: 1) | NP_989545.3) | KPEEHPHHHGHHHGNPHEEEHVRAPS |
| | | | | GHHQAGRCLLWACKACKRKTTNAD |
| | | | | RRKAATMRERRRLSKVNEAFETLKRC |
| | | | | TSTNPNQRLPKVEILRNAIRYIESLQAL |
| | | | | LREQEDAYYPVLEHYSGESDASSPRS |
| | | | | NCSDGMMEYSGPPCSSRRRNSYDSSY |
| | | | | YTESPNDPKHGKSSVVSSLDCLSSIVE |
| | | | | RISTDNSTCPILPPAEAVAEGSPCSPQE |
| | | | | GANLSDSGAQIPSPTNCTPLPQESSSSS |
| | | | | SSNPIYQVL (SEQ ID NO: 1) |
| MyoD | Anas | NP_001297287.1 | NM_001310358.1 | MDLLGAMEMPEGSLCSFTGADDFYD |
| | platyrhynchos | (SEQ ID | (encodes) | DPCFNTSDMHFFEDLDPRLVHVGGLL |
| | (Duck) | NO: 2) | NP_001297287.1) | KPEEHPHHHGHHHHGHPHEEEHVRA |
| | | | | PSGHHQAGRCLLWACKACKRKTTNA |
| | | | | DRRKAATMRERRRLSKVNEAFETLKR |
| | | | | CTSTNPNQRLPKVEILRNAIRYIESLQA |
| | | | | LLREQEDVYYPVLEHYSGESDASSPRS |
| | | | | NCSDGMMEYSGPPCSSRRRNSYDSSY |
| | | | | YTESPNDPKHGKSSVVSSLDCLSGIVE |
| | | | | RISTDNSTCPILPPAETVAEGSPCSPQE |
| | | | | GASLNDSGAQIPSPTNCTPLPQESSSSN |
| | | | | PIYQVL (SEQ ID NO: 2) |
| MyoD | Meleagris | NP_001290100.1 | NM_001303171.1 | MDLLGPMEMTEGSLCSFAAADDFYD |
| | gallopavo | (SEQ ID | (encodes | DPCFNTSDMHFFEDLDPRLVHVGGLL |
| | (Turkey) | NO: 3) | NP_001290100.1) | KPEEHPHHHGHHHGHPHEEEHVRAPS |
| | | | | GHHQAGRCLLWACKACKRKTTNAD |
| | | | | RRKAATMRERRRLSKVNEAFETLKRC |
| | | | | TSTNPNQRLPKVEILRNAIRYIGSLQAL |
| | | | | LREQEDAYYPVLEHYSGESDASSPRS |
| | | | | NCSDGMMEYSGPPCSSRRRNSYDSSY |
| | | | | YTESPNDPKHGKSSVVSSLDCLSSIVE |
| | | | | RISTDNSTCPILPPAEAVAEGSPCSPQE |
| | | | | GASLNDSGAQIPSPTNCTPLPQDSSSS |
| | | | | NNPIYQVL (SEQ ID NO: 3) |
| MyoD | Sus scrofa | NP_001002824.1 | NM_001002824.1 | MELLSPPLRDVDLTGPDGSLCNFATA |
| | (Porcine) | (SEQ ID | (encodes) | DDFYDDPCFDSPDLRFFEDLDPRLVH |
| | | NO: 4) | NP_001002824.1 | VGALLKPEEHSHFPAAAHPAPGARED |
| | | | | EHVRAPSGHHQAGRCLLWACKACKR |
| | | | | KTTNADRRKAATMRERRRLSKVNEA |
| | | | | FETLKRCTSSNPNQRLPKVEILRNAIR |
| | | | | YIEGLQALLRDQDAAPPGAAAAFYAP |
| | | | | GPLPPGRGGEHYSGDSDASSPRSNCS |
| | | | | DGMMDYSGPPSGARRRNCYDGTYYS |
| | | | | EAPSEPRPGKNAAVSSLDCLSSIVESIS |
| | | | | TESPAAPALLLADTPRESSPGPQEAAA |
| | | | | GSEVERGTPTPSPDAAPQCPASANPNP |
| | | | | IYQVL (SEQ ID NO: 4) |
| MyoD | Bos taurus | NP_001035568.2 | NM_001040478.2 | MELLSPPLRDVDLTGPDGSLCNFATA |
| | (Bovine) | (SEQ ID | (encodes | DDFYDDPCFDSPDLRFFEDLDPRLVH |
| | | NO: 5) | NP_001035568.2) | VGALLKPEEHSHFPAAAHPAPGARED |
| | | | | EHVRAPSGHHQAGRCLLWACKACKR |
| | | | | KTTNADRRKAATMRERRRLSKVNEA |
| | | | | FETLKRCTSSNPNQRLPKVEILRNAIR |
| | | | | YIEGLQALLRDQDAAPPGAAAAFYAP |
| | | | | GPLPPGRSGEHYSGDSDASSPRSNCSD |
| | | | | GMMDYSGPPSGARRRNCYDRTYYSE |
| | | | | APNEPRPGKSAAVSSLDCLSSIVERIST |
| | | | | ESPAAPALLLADAPPESSPGPQEAAGS |
| | | | | EVERGTPAPSPDTAPQGLAGANPNPIY |
| | | | | QVL (SEQ ID NO: 5) |
| MyoD | Oreochromis | NP_001266649.1 | NM_001279720.1 | MELSDISFPIPTADDFYDDPCFNTSDM |
| | niloticus | (SEQ ID | (encodes | HFFEDLDPRLVHVGLLKPDDSSSSSSS |
| | (Tilapia) | NO: 6) | NP_001266649.1) | SPSSSSSSPSSLLHLHHHAEVEDDEHV |
| | | | | RAPSGHHQAGRCLLWACKACKRKTT |
| | | | | NADRRKAATLRERRRLSKVNDAFETL |
| | | | | KRCTTANPNQRLPKVEILRNAISYIESL |
| | | | | QALLRGGQEDGFYPVLEHYSGDSDAS |
| | | | | SPRSNCSDGMTDFNGPTCQTTRRGSY |
| | | | | DSSSYFSETPNGGLKSERSSVVSSLDC |
| | | | | LSSIVERISTDNSSLLPPADGPGSPTTTT |
| | | | | TVPVGEAGTAPATAQVSSPTASQDPN |
| | | | | LIYQVL (SEQ ID NO: 6) |

MYOG

| | | | | |
|---|---|---|---|---|
| MYOG | Gallus | NP_989515.1 | NM_204184.2 | MELFETNPYFFPEQRFYDGENFLGSRL |
| | gallus | (SEQ ID | (encodes | QGYEAAAFPERPEVTLCPESRGALEE |
| | (chicken) | NO: 7) | NP_989515.1) | KDSTLPEHCPGQCLPWACKICKRKTV |
| | | | | SIDRRRAATLREKRRLKKVNEAFEAL |
| | | | | KRSTLLNPNQRLPKVEILRSAIQYIERL |
| | | | | QSLLSSLNQQEREQRELRYRPAAPQP |
| | | | | AAPSECGSGSSSCSPEWSTQLEFGTNP |
| | | | | ADHLLSDDQAEDRNLHSLSSIVESIAV |
| | | | | EDVAVTFPEERVQN (SEQ ID NO: 7) |
| MYOG | Anas | NP_001297305.1 | NM_001310376.1 | MELFETNPYFFPDQRFYDGENFLGSRL |
| | platyrhynchos | (SEQ ID | (encodes | QGYEPAAFPEQPEVALCPESRVALEE |
| | (Duck) | NO: 8) | NP_001297305.1) | KDSSLAEHCPGQCLPWACKICKRKTV |
| | | | | SIDRRRAATLREKRRLKKVNEAFEAL |
| | | | | KRSTLLNPNQRLPKVEILRSAIQYIERL |
| | | | | QSLLSTLNQQEREQRDLRYRPAGPQP |
| | | | | AAASECGSGSSSCSPEWSTQLEFGTNP |
| | | | | TDHLLPDEAAEDRNLHSLSSIVESIAV |
| | | | | EDVAVAFQEEQVQN (SEQ ID NO: 8) |
| MYOG | Meleagris | NP_001290099.1 | NM_001303170.1 | MELFETNPYFFPEQRFYDGENFLGSRL |
| | gallopavo | (SEQ ID | (encodes | QSYEAAAFPERPEVALCPESRGALEEK |
| | (Turkey) | NO: 9) | NP_001290099.1) | DSMLPEHCPGQCLPWACKICKRKTVS |
| | | | | IDRRRAATLREKRRLKKVNEAFEALK |
| | | | | RSTLLNPNQRLPKVEILRSAIQYIERLQ |
| | | | | SLLSSLNQQEREQRELRYRPAAPQPA |
| | | | | APSECGSGSSSCSPEWSTQLEFSTNPA |
| | | | | DHLLSDDAAEDRNLHSLSSIVESIAVE |
| | | | | DVAVTFPEERVQN (SEQ ID NO: 9) |
| MYOG | Sus scrofa | NP_001012406.1 | NM_001012406.1 | MELYETSPYFYQEPHFYDGENYLPVH |
| | (Porcine) | (SEQ ID | (encodes | LQGFEPPGYERTELSLSPEARVPLEDK |
| | | NO: 10) | NP_001012406.1) | GLGTPEHCPGQCLPWACKVCKRKSVS |
| | | | | VDRRRAATLREKRRLKKVNEAFEAL |
| | | | | KRSTLLNPNQRLPKVEILRSAIQYIECL |
| | | | | QALLSSLNQEERDLRYRGGGGPQPGV |
| | | | | PSECSSHSASCSPEWGSALEFGPNPGD |
| | | | | HLLTADPTDAHNLHSLTSIVDSITVED |
| | | | | VAVAFPDETMPN (SEQ ID NO: 10) |
| MYOG | Bos taurus | NP_001104795.1 | NM_001111325.1 | MELYETSPYFYQEPHFYDGENYLPVH |
| | (Bovine) | (SEQ ID | (encodes | LQGFEPPGYERAELSLSPEARVPLEDK |
| | | NO: 11) | NP_001104795.1) | GLGPAEHCPGQCLPWACKVCKRKSV |
| | | | | SVDRRRAATLREKRRLKKVNEAFEAL |
| | | | | KRSTLLNPNQRLPKVEILRSAIQYIERL |
| | | | | QALLSSLNQEERDLRYRGGGGPQPAV |
| | | | | PSECSSHSASCSPQWGSALEFGPNPGD |
| | | | | HLLPADPTDAHNLHSLTSIVDSITVED |
| | | | | VAAAFPDETIPN (SEQ ID NO: 11) |
| MYOG | Oreochromis | NP_001266455.1 | NM_001279526.1 | MELFETNPYFFPDQRFYEGGDSYFPSR |
| | niloticus | (SEQ ID | (encodes | LPGAYDQAGYQDRNSMMGLCGNLSG |
| | (Tilapia) | NO: 12) | NP_001266455.1) | GVGVGVTGTEDKASPSSM |
| | | | | SPHSEPHCPGQCLPWACKLCKRKTVT |
| | | | | MDRRRAATLREKRRLKKVNEAFDAL |
| | | | | KRSTLMNPNQRLPKVEILR |
| | | | | SAIQYIERLQALVSSLNQQDTETGQQG |
| | | | | LLYRPSPTQPRVSSSSEPSSGSTCCSSP |
| | | | | EWSSTPEQCTQSYSS |
| | | | | EDLLSAADSPEQGNMRALTSIVDSISA |
| | | | | ADGPVAFPVDIPK (SEQ ID NO: 12) |

Pax 7

| | | | | |
|---|---|---|---|---|
| PAX7 | Gallus | NP_990396.1 | NM_205065.1 | MAALPGTVPRMMRPAPGQNYPRTGF |
| | gallus | (SEQ ID | (encodes | PLEVSTPLGQGRVNQLGGVFINGRPLP |
| | (chicken) | NO: 13) | NP_990396.1) | NHIRHKIVEMAHHGIRPCVISRQLRVS |
| | | | | HGCVSKILCRYQETGSIRPGAIGGSKP |
| | | | | RQVATPDVEKKIEEYKRENPGMFSWE |
| | | | | IRDRLLKDGHCDRSTVPSVSSISRVLRI |
| | | | | KFGKKEEEEDCDKKEEDGEKKAKHSI |
| | | | | DGILGDKGNRLDEGSDVESEPDLPLK |
| | | | | RKQRRSRTTFTAEQLEELEKAFERTH |
| | | | | YPDIYTREELAQRTKLTEARVQVWFS |
| | | | | NRRARWRKQAGANQLAAFNHLLPGG |
| | | | | FPPTGMPTLPPYQLPDSTYPTTTISQDG |
| | | | | GSTVHRPQPLPPSTMHQGGLAAAAAA |
| | | | | DSSSAYGARHSFSSYSDSFMNAAAPA |
| | | | | NHMNPVSNGLSPQKQGAQNKMQCSR |
| | | | | WNLTIALNNQVMSILSNPSGVPPQPQ |
| | | | | ADFSISPLHGGLDTTNSISASCSQRSDS |
| | | | | IKSVDSLPTSQSYCPPTYSTTSYSVDPV |
| | | | | AGYQYGQYGQTAVDYLTKNVSLSTQ |
| | | | | RRMKLGEHSAVLGLLPVETGQAY |
| | | | | (SEQ ID NO: 13) |
| PAX7 | Anas | NP_001297324.1 | NM_001310395.1 | MAALPGTVPRMMRPAPGQNYPRTGF |
| | platyrhynchos | (SEQ ID | (encodes | PLEVSTPLGQGRVNQLGGVFINGRPLP |
| | (Duck) | NO: 14) | NP_001297324.1) | NHIRHKIVEMAHHGIRPCVISRQLRVS |
| | | | | HGCVSKILCRYQETGSIRPGAIGGSKP |
| | | | | RVATPDVEKKIEEYKRENPGMFSWEI |
| | | | | RDRLLKDGHCDRSTVPSGLVSSISRVL |
| | | | | RIKFGKKEEEEDCDKKEEDGEKKAKH |
| | | | | SIDGILGDKGNRLDEGSDVESEPDLPL |
| | | | | KRKQRRSRTTFTAEQLEELEKAFERT |
| | | | | HYPDIYTREELAQRTKLTEARVQVWF |
| | | | | SNRRARWRKQAGANQLAAFNHLLPG |
| | | | | GFPPTGMPTLPPYQLPDSTYPTTTISQD |
| | | | | GGSTVHRPQPLPPSTMHQGGLAAAAA |
| | | | | ADTGSAYGARHSFSSYSDSFMNAAAP |
| | | | | ANHMNPVSNGLSPQVMSILSNPSGVP |
| | | | | PQPQADFSISPLHGGLDTTNSISASCSQ |
| | | | | RSDSIKSVDSLPTSQSYCPPTYSTTSYS |
| | | | | VDPVAGYQYGQYGQTAVDYLAKNV |
| | | | | SLSTQRRMKLGGHSAVLGLLPVETGQ |
| | | | | AY (SEQ ID NO: 14) |
| PAX7 | Meleagris | NP_001290114.1 | NM_001303185.1 | MAALPGTVPRMMRPAPGQNYPRTGF |
| | gallopavo | (SEQ ID | (encodes | PLEVSTPLGQGRVNQLGGVFINGRPLP |
| | (Turkey) | NO: 15) | NP_001290114.1) | NHIRHKIVEMAHHGIRPCVISRQLRVS |
| | | | | HGCVSKILCRYQETGSIRPGAIGGSKP |
| | | | | RQVATPDVEKKIEEYKRENPGMFSWE |
| | | | | IRDRLLKDGHCDRSTVPSVSSISRVLRI |
| | | | | KFGKKEEEEDCDKKEEDGEKKAKHSI |
| | | | | DGILGDKGNRLDEGSDVESEPDLPLK |
| | | | | RKQRRSRTTFTAEQLEELEKAFERTH |
| | | | | YPDIYTREELEKAFERTHYPDIYTREE |
| | | | | LAQRTKLTEARVQVWFSNRRARWRK |
| | | | | QAGANQLAAFNHLLPGGFPPTGMPTL |
| | | | | PPYQLPDSTYPTTTISQDGSSTVHRPQP |
| | | | | LPPSTMHQGGLAAAAAADSSSAYGA |
| | | | | RHSFSSYSDSFMNAAAPANHMNPVSN |
| | | | | GLSPQVMSILSNPSGVPPQPQADFSISP |
| | | | | LHGGLDTTNSISASCSQRSDSIKSVDSL |
| | | | | PTSQSYCPPTYSTTSYSVDPVAGYQY |
| | | | | GQYGQTAVDYLAKNVSLSTQRRMKL |
| | | | | GEHSAVLGLLPVETGQAY (SEQ ID |
| | | | | NO: 15) |
| PAX7 | Sus scrofa | XP_020951119.1 | XM_021095460.1 | MAALPGTVPRMMRPAPGQNYPRTGF |
| | (Porcine) | (SEQ ID | (encodes | PLEVSTPLGQGRVNQLGGVFINGRPLP |
| | | NO: 16) | XP_020951119.1) | NHIRHKIVEMAHHGIRPCVISRQLRVS |
| | | | | HGCVSKILCRYQETGSIRPGAIGGSKP |
| | | | | RQVATPDVEKKIEEYKRENPGMFSWE |
| | | | | IRDRLLKDGHCDRSTVPSVSSISRVLRI |
| | | | | KFGKKEEDDEADKKEDDSEKKAKHSI |
| | | | | DGILGDKGNRLDEGSDVESEPDLPLK |
| | | | | RKQRRSRTTFTAEQLEELEKAFERTH |
| | | | | YPDIYTREELAQRTKLTEARVQVWFS |
| | | | | NRRARWRKQAGANQLAAFNHLLPGG |
| | | | | FPPTGMPTLPPYQLPDSTYPTTTISQDG |
| | | | | GSTVHRPQPLPPSTMHQGGLAAAAAA |
| | | | | ADTSSAYGARHSFSSYSDSFMNPAAP |
| | | | | SNHMNPVSNGLSPQVMSILSNPSAVPP |
| | | | | QPQADFSISPLHGGLDSATSISASCSQR |
| | | | | ADSIKPGDSLPTSQSYCPPTYSTTGYS |
| | | | | VDPVAGYQYGQYGQTAVDYLAKNV |
| | | | | SLSTQRRMKLGEHSAVLGLLPVETGQ |
| | | | | AY (SEQ ID NO: 16) |
| PAX7 | Bos taurus | XP_015316176.1 | XM_015460690.2 | MAALPGTVPRMVRPAPGQNYPRTGFP |
| | (Bovine) | (SEQ ID | (encodes | LEVSTPLGQGRVNQLGGVFINGRPLP |
| | | NO: 17) | XP_015316176.1) | NHIRHKIVEMAHHGIRPCVISRQLRVS |
| | | | | HGCVSKILCRYQETGSIRPGAIGGSKP |
| | | | | RQVATPDVEKKIEEYKRENPGMFSWE |
| | | | | IRDRLLKDGHCDRSTVPSVSSISRVLRI |
| | | | | KFGKKEEEDEADKKEEDGEKKAKHSI |
| | | | | DGILGDKGNRLDEGSDVESEPDLPLK |
| | | | | RKQRRSRTTFTAEQLEELEKAFERTH |
| | | | | YPDIYTREELAQRTKLTEARVQVWFS |
| | | | | NRRARWRKQAGANQLAAFNHLLPGG |
| | | | | FPPTGMPTLPPYQLPDSTYPTTTISQDG |
| | | | | GSTVHRPQPLPPSTMHQGGLAAAAAA |
| | | | | ADTSSAYGARHGFSSYSDSFMNPAAA |
| | | | | SNHMNPVSNGLSPQWSLTVLFGVQG |
| | | | | HHLHLIGAED (SEQ ID NO: 17) |
| PAX7 | Oreochromis | XP_025763203.1 | XM_025907418.1 | MATLPGTVPRMVRPAPGQNYPRTGFP |
| | niloticus | (SEQ ID | (encodes | LEVSTPLGQGRVNQLGGVFINGRPLP |
| | (Tilapia) | NO: 18) | XP_025763203.1) | NHIRHKIVEMAHHGIRPCVISRQLRVS |
| | | | | HGCVSKILCRYQETGSIRPGAIGGSKP |
| | | | | RQVATPDVEKKIEEYKRENPGMFSWE |
| | | | | IRDKLLKDGVCDRSTVPSGNRTDDAS |
| | | | | DVDSEPDLPLKRKQRRSRTTFTAEQLE |
| | | | | ELEKAFERTHYPDIYTREELAQRTKLT |
| | | | | EARVQVWFSNRRARWRKQAGANQL |
| | | | | AAFNHLLPGGFPPTGMPTLPTYQLPES |
| | | | | SYPSTTLSQEGSSTLHRPQPLPPSSMH |
| | | | | QGGLSADSSSAYGLSSNRHSFSSYSDT |
| | | | | FMSPSASSNHMNPVGNGLSPQVMSIL |
| | | | | SNPSAVPSQPQHDFSISPLHSSLESSNPI |
| | | | | SASCSQRSDTIKSVDSLASSQSYCPPT |
| | | | | YSATSYSVDPVTAGYQYSQYGQTAV |
| | | | | DYLAKNVSLSTQRRMKLGDHSAVLG |
| | | | | LLQVETGQAY (SEQ ID NO: 18) |

Pax3

| | | | | |
|---|---|---|---|---|
| PAX3 | Gallus | NP_001384688.1, | NM_001397759.1 | MTTLAGAVPRMMRPGAGQSYPRGGF |
| | gallus | (SEQ ID | (encodes | PLEVSTPLGQGRVNQLGGVFINGRPLP |
| | (chicken) | NO: 19) | NP_001384688.1) | NHIRHKIVEMAHHGIRPCVISRQLRVS |
| | | | | HGCVSKILCRYQETGSIRPGAIGGSKP |
| | | | | KVTTPDVEKKIEEYKRENAGMFSWEI |
| | | | | RDRLLKDGVCDRNTVPSVSSISRILRS |
| | | | | KFGKGEEEEAELERKEAEEGDKKAKH |
| | | | | SIDGILSERASAAQSDEGSDIDSEPDLP |
| | | | | LKRKQRRSRTTFTAEQLEELERAFERT |
| | | | | HYPDIYTREELAQRAKLTEARVQVWF |
| | | | | SNRRARWRKQAGANQLMAFNHLIPG |
| | | | | GFPPSAMPTLPTYQLSEPSYQPTSIPQA |
| | | | | VSDPSSTVHRPQPLPPSTVHQSSLPSNP |
| | | | | ESSSAYCLPSTRHGFSSYTDSFVPPSGP |
| | | | | SNPMNPAIGNGLSPQVMGLLTNHGGV |
| | | | | PHQPQTDYALSPLTGGLEPTTTVSASC |
| | | | | SQRLDHMKSLDSLPTSQSYCPPTYSTT |
| | | | | GYSMDPVTGYQYGQYGQSAFHYLKP |
| | | | | DIA (SEQ ID NO: 19) |
| PAX3 | Anas | NP_001297282.1 | NM_001310353.1 | MTTLAGAVPRMMRPGAGQNYPRSGF |
| | platyrhynchos | (SEQ ID | (encodes | PLEVSTPLGQGRVNQLGGVFINGRPLP |
| | (Duck) | NO: 20) | NP_001297282.1) | NHIRHKIVEMAHHGIRPCVISRQLRVS |
| | | | | HGCVSKILCRYQETGSIRPGAIGGSKP |
| | | | | KQVTTPDVEKKIEEYKRENAGMFSWE |
| | | | | IRDKLLKDGVCDRNTVPSVSSISRILRS |
| | | | | KFGKGEEEEAELERKEAEEGDKKAKH |
| | | | | SIDGILSERAPQSDEGSDIDSEPDLPLK |
| | | | | RKQRRSRTTFTAEQLEELERAFERTHY |
| | | | | PDIYTREELAQRAKLTEARVQVWFSN |
| | | | | RRARWRKQAGANQLMAFNHLIPGGF |
| | | | | PPSAMPTLPTYQLSEPSYQPTSIPQAVS |
| | | | | DPSSTVHRPQPLPPSTVHQSSLPSNPES |
| | | | | STAYCLPSTRHGFSSYTDSFVPPSGPS |
| | | | | NPMNPAIGNGLSPQVMGLLTNHGGVP |
| | | | | HQPQTDYALSPLTGGLEPTTTVSASCS |
| | | | | QRLEHMKSLDSLPTSQSYCPPTYSTTG |
| | | | | YSMDPVTGYQYGQYGQSAFHYLKPD |
| | | | | IA (SEQ ID NO: 20) |
| PAXBP1 | Meleagris | XP_010721593.1 | XM_010723291.1 | MDTICSISTSLGDSSFRLLDSVLQEHYS |
| (PAX3 | gallopavo | (SEQ ID | (encodes | IALLVCSVCNDSVLVRRPKEKKRARE |
| homolog) | (Turkey) | NO: 21) | XP_010721593.1) | NKEVPRASLLSFQDEEEETEEVFKVK |
| | | | | KSSYSKKIVKQLKKEYKEDLEKSKVR |
| | | | | TEVNSPTDVEPPLEKTGQIKDIGQEDG |
| | | | | TANSEHGEEEMEVESEKEEEKPKPGG |
| | | | | AFSSALSSLNVLRPGEIPDAAFIHAAR |
| | | | | KKRQMARELGDFTPVDSEPGKSRLVR |
| | | | | EDENDASDDEDDDEKRRIVFTVKEKS |
| | | | | QRQKIAEEIGIEGSDDEALVAGEQDEE |
| | | | | LSRWEQEQIRKGINIPQVQPSQPAEVN |
| | | | | NLYYQNTYQTLSYGSSYGIPYTYAAY |
| | | | | GSSEAKSQKTDNTVPFKTPSNEMTPIT |
| | | | | IDLVKKQLKDRLDSMKELHKANRQQ |
| | | | | FEKHQQSQEDSTKAIERLEGSSGGIGE |
| | | | | QYKFLQEMRGYVQDLLECFSEKVPLI |
| | | | | NELESAMHQLYKQRASRLVQRRQDDI |
| | | | | KDESSEFSSHSNKALMAPNLDSFGRD |
| | | | | RVLYQEQVKRRTAEREARRARRRQA |
| | | | | REQTGKMADHLEGLSSDDEETSTDIT |
| | | | | NFNMERDRILKESSKVFEDVLESFYSI |
| | | | | DCIKSQFEAWRSKYFASYKDAYIGLC |
| | | | | LPKLFNPLIRLQLLVWTPLEGKCRDFE |
| | | | | TMLWFESLLFYGCEEQEQEKDDADIS |
| | | | | LLPTIVERVVLPKLTVISENIWDPFSTT |
| | | | | QTSRMVAIVQKLVNGYPSVVNAENK |
| | | | | NTQMLLKALLLRMRRTLDDDVFMPL |
| | | | | YPKNVLENKNSGPYLFFQRQFWSSVK |
| | | | | LLGNFLQWYGILSNKTLQELSIDGLLN |
| | | | | RYILMAFQNSEYGDDSIKKAQSVIACF |
| | | | | PKQWFANLKGDKTISQLENFCRYLVH |
| | | | | LADTIYRNSIGCSDVEKRNARVANKA |
| | | | | DHKASSKYTSTGSCCDSCK (SEQ ID |
| | | | | NO: 21) |
| PAX3 | Sus scrofa | XP_020931017.1 | XM_021075358.1 | MTTLAGAVPRMMRPGPGQNYPRSGF |
| | (Porcine) | (SEQ ID | (encodes | PLEVSTPLGQGRVNQLGGVFINGRPLP |
| | | NO: 22) | XP_020931017.1) | NHIRHKIVEMAHHGIRPCVISRQLRVS |
| | | | | HGCVSKILCRYQETGSIRPGAIGGSKP |
| | | | | KVTTPDVEKKIEEYKRENPGMFSWEI |
| | | | | RDKLLKDAVCDRNTVPSVSSISRILRS |
| | | | | KFGKGEEEEADLERKEAEESEKKTKH |
| | | | | SIDGILSERASAPQSDEGSDIDSEPDLP |
| | | | | LKRKQRRSRTTFTAEQLEELERAFERT |
| | | | | HYPDIYTREELAQRAKLTEARVQVWF |
| | | | | SNRRARWRKQAGANQLMAFNHLIPG |
| | | | | GFPPTAMPTLPTYQLSETSYQPTAIPQ |
| | | | | AVSDPSSTVHRPQPLPPSTVHQSTIPSN |
| | | | | PDSSSAYCLPSTRHGFSSYTDSFVPPS |
| | | | | GPSNPMNPAIGNGLSPQVMGLLTNHG |
| | | | | GVPHQPQTDYALSPLTGGLEPTTTVS |
| | | | | ASCSQRLEHMKSLDSLPTSQSYCPPTY |
| | | | | STTGYSMDPVTGYQYGQYGQSAFHY |
| | | | | LKPDIA (SEQ ID NO: 22) |
| PAX3 | Bos taurus | NP_001193747.1 | NM_001206818.2 | MTTLAGAVPRMMRPGPGQNYPRSGF |
| | (Bovine) | (SEQ ID | (encodes | PLEVSTPLGQGRVNQLGGVFINGRPLP |
| | | NO: 23) | NP_001193747.1) | NHIRHKIVEMAHHGIRPCVISRQLRVS |
| | | | | HGCVSKILCRYQETGSIRPGAIGGSKP |
| | | | | KQVTTPDVEKKIEEYKRENPGMFSWE |
| | | | | IRDKLLKDAVCDRNTVPSVSSISRILRS |
| | | | | KFGKGEEEEADLERKEAEESEKKAKH |
| | | | | SIDGILSERASAPQSDEGSDIDSEPDLP |
| | | | | LKRKQRRSRTTFTAEQLEELERAFERT |
| | | | | HYPDIYTREELAQRAKLTEARVQVWF |
| | | | | SNRRARWRKQAGANQLMAFNHLIPG |
| | | | | GFPPTAMPTLPTYQLSETSYQPTAIPQ |
| | | | | AVSDPSSTVHRPQPLPPSTVHQSTLPS |
| | | | | NPDSSSAYCLPSTRHGFSSYTDSFVPP |
| | | | | SGPSNPMNPAIGNGLSPQVMGLLTNH |
| | | | | GGVPHQPQTDYALSPLTGGLEPTTTV |
| | | | | SASCSQRLDHMKSLDSLPTSQSYCPPT |
| | | | | YSTTGYSMDPVTGYQYGQYGQSAFH |
| | | | | YLKPDIA (SEQ ID NO: 23) |
| PAX3 | Oreochromis | XP_005461779.1 | XM_005461722.3 | MTALAGSIPRMMRPSLTQNYPRSGFP |
| | niloticus | (SEQ ID | (encodes | LEVSTPLGQGRVNQLGGVFINGRPLP |
| | (Tilapia) | NO: 24) | XP_005461779.1) | NHIRHKIVEMAHHGIRPCVISRQLRVS |
| | | | | HGCVSKILCRYQETGSIRPGAIGGSKP |
| | | | | KGTSPDVERRIEEYKRENPVSAISRIM |
| | | | | RSKFGGKGDEEEDEDEIEKKELEDNE |
| | | | | RRAKHSIEGILGDRSSHSDEGSEVESEP |
| | | | | DLPLKRKQRRSRTTFTAEQLEELERAF |
| | | | | ERTHYPDIYTREELAQRAKLTEARVQ |
| | | | | VWFSNRRARWRKQAGANQLMAFNH |
| | | | | LIPGGFPPSAMSSIPPYQLSDSSYPPSSI |
| | | | | AQVSEPPSTVHRPQPLPPSSGHQASGG |
| | | | | GGQGEGGSAYCLASGRHSFSGYSDSF |
| | | | | VSPGAPTNPMNPSIGNGLSPQVMGLL |
| | | | | NPGGVPHQPQSDYAISPLTGGLEPPAG |
| | | | | MAASCSQRMDHIKGLEGLTSVPSMSA |
| | | | | LPSLPSSQSYCPPSYSSPGYTVDHVAS |
| | | | | YQYSQYGQSTLPYLRPEIT (SEQ ID |
| | | | | NO: 24) |

MEF2b

| | | | | |
|---|---|---|---|---|
| MEF2b | Gallus | XP_430389.3, | XM_430389.7 | MGRKKIQISRILDQRNRQVTFTKRKFG |
| | gallus | isoform | (encodes | LMKKAYELSVLCDCEIALIIFNSTNRL |
| | (chicken) | X1 (SEQ ID | XP_430389.3) | FQYASTDMDKVLLKYTEYSEPHESRT |
| | | NO: 25) | | NSDILETLKRKGLGLESHELELEEGLD |
| | | | | PGEKLRQMNEGMDLTVARPRFYSPVP |
| | | | | LPEVPYGSSPSSSSDGALGSASSSPQSQ |
| | | | | GRPPAFKPTAPKPSGRSPGPMPPGIGY |
| | | | | PLFPAGSLNRALATKTPPPLYLGADG |
| | | | | QRRSGGSTARPLYSGLQTLNPVLASG |
| | | | | SASIPSHSLTGFPFLAPAQAAEFGAGE |
| | | | | APPPPGFLQPGPPAPWQPPRDMAALG |
| | | | | ASSRIVPTEDLAPGSSPQPHAISIKSER |
| | | | | VSPGLGCPSGAPQPSQGSLTSLSEAPQ |
| | | | | GAADLQPRDDYSKGYPYPPPPPRPLA |
| | | | | EEQRATAAVPVPARRAQAEDAWQR |
| | | | | (SEQ ID NO: 25) |
| MEF2b | Anas | XP_038025156.1 | XM_038169228.1 | MGRKKIQISRILDQRNRQVTFTKRKFG |
| | platyrhynchos | (SEQ ID | (encodes | LMKKAYELSVLCDCEIALIIFNSTNRL |
| | (Duck) | NO: 26) | XP_038025156.1) | FQYASTDMDKVLLKYTEYSEPHESRT |
| | | | | NSDILETLKRKGLGLDSHELELDEGLD |
| | | | | GGEKMRKLNEGMDLTVARPRFYSPV |
| | | | | PLPEASYSSSPPAGGDGALGSTSSSPQ |
| | | | | SQGRPPAFKPSAPKLSGRSPGPMPPGI |
| | | | | GYPLFPPGSLNRALATKTPPPLYLGAD |
| | | | | GRRGEAHGSLASGRSGGSAARPLYPG |
| | | | | LQTLSPVLPPGSAGIPNHSLSGFPFLAP |
| | | | | AQAEYGAGEAPPPPGFLQPGPPASWQ |
| | | | | PPRDMAALGTSTRIVPAEDAAPGSSPQ |
| | | | | HHAISIKSERVSPGLGCPSGTPQPPPGG |
| | | | | LTSLNEAPRGSGDLQPRDDYAKGYPY |
| | | | | PLGPPRPLAEEQRATTVPVPPRRAQAV |
| | | | | DGWQR (SEQ ID NO: 26) |
| MEF2b | Meleagris | XP_010723330.1 | M_010725028.3 | MGRKKIQISRILDQRNRQVTFTKRKFG |
| | gallopavo | (SEQ ID | (encodes | LMKKAYELSVLCDCEIALIIFNSTNRL |
| | (Turkey) | NO: 27) | XP_010723330.1) | FQYASTDMDKVLLKYTEYSEPHESRT |
| | | | | NSDILETLKRKGLGLESHELELEEGLD |
| | | | | PGEKLRQLNEGMDLTVARPRFYSPVP |
| | | | | LPEVPYGSSPPSSSDGALGSASSSPQSQ |
| | | | | GRPPTFKPTAPKPSGRSPGPMPPGIGY |
| | | | | PLFPAGSLNRALATKTPPPLYLGADG |
| | | | | QRRSGSGAARPLYSGLQTLSPVLASGS |
| | | | | AGIPSHSLTGFPFLTPAQAEFGAGEAP |
| | | | | PPPGFLQPGPPAPWQPPRDMAALGAS |
| | | | | SRIIPTEDLAPGSSPQPHTISIKSERVSP |
| | | | | GLGCPSGTPQPSPGSLTSLSEAPRGAA |
| | | | | DLQPRDDYTKGYPYPLPPPRPLAEEQ |
| | | | | RATATIPVPARRVQAEDAWQR (SEQ |
| | | | | ID NO: 27) |
| MEF2b | Sus scrofa | XP_020939100.1 | XM_021083441.1 | MPRPISSRGAGPPALKAPVAAGSSEFC |
| | (Porcine) | (SEQ ID | (encodes | AATAAARCVRERQSECPGGADQGYS |
| | | NO: 28) | XP_020939100.1) | TQPGTMGRKKIQISRILDQRNRQVTFT |
| | | | | KRKFGLMKKAYELSVLCDCEIALIIFN |
| | | | | SANRLFQYASTDMDRVLLKYTEYSEP |
| | | | | HESRTNTDILETLKRRGVGLDGPELEP |
| | | | | DEGLEGPGEKLRRLAGDGGDPALPRP |
| | | | | RLYPAAPTMPSPDMVYGALPPPGCDP |
| | | | | SGLGEALPAQSRPSPFRPAAPKAGPPG |
| | | | | LAHPLFSPSHLASKTPPPLYLATDGRR |
| | | | | PDLPGGLAGARGGLSTSRGLYGSLQS |
| | | | | PCSTATPGTPLGSFPFLPAGPPEYGLG |
| | | | | DPPPPPGLLQPPTLAPWQPSRGDGPTA |
| | | | | TPTQSSGGRSLGEEGPPARGASSPTPP |
| | | | | VSIKSERLSPAPGGPGDFPKTFPYPLLL |
| | | | | ARPLAEPLRPGPPMRRLPTADGWPR |
| | | | | (SEQ ID NO: 28) |
| MEF2b | Bos taurus | NP_001139265.1 | NM_001145793.1 | MGRKKIQISRILDQRNRQVTFTKRKFG |
| | (Bovine) | (SEQ ID | (encodes | LMKKAYELSVLCDCEIALIIFNSSNRLF |
| | | NO: 29) | NP_001139265.1) | QYASTDMDRVLLKYTEYSEPHESRTI |
| | | | | TDILETLKRRGVGLDGPELEPDEGLEG |
| | | | | PGEKLRRLAGDGGDPALPRPRLYPAA |
| | | | | PTMPSPDMVYGALPPPACEPTGLGEA |
| | | | | LPAQSRPSPFRPAAPKAGPPGLAHPLF |
| | | | | SPSHLASKTPPPLYLAADGRRPDLPGG |
| | | | | LAGTRGGLSSSRGLYGGLQSPCSTAA |
| | | | | PGPPLGSFPFLPAGPPEYGLGDPPPPPG |
| | | | | LLQPPTLAPWQPSRADGPPATPTQPSG |
| | | | | GRSLGEDGPPARGASSPTPPVSIKSERL |
| | | | | SPAPGGPGDFPKTFPYPLLLARPLAEP |
| | | | | LRPGPPLRRLPTADGWPR (SEQ ID |
| | | | | NO: 29) |
| MEF2b | Oreochromis | XP_005479045.1 | XM_005478988.4 | MGRKKIQISRILDQRNRQVTFTKRKFG |
| | niloticus | (SEQ ID | (encodes | LMKKAYELSVLCDCEIALIIFNSTNRL |
| | (Tilapia) | NO: 30) | XP_005479045) | FQYASTDMDKVLLKYTEYSEPHESRT |
| | | | | NTDILETLRRKGLGLDGSELDSEESMQ |
| | | | | VATDKYPLSEGMDLSVARQRFYGPSL |
| | | | | LSPEAQFLVSAGCENGFPNSSGSGMA |
| | | | | SHRPPSFKSLNSRSSSASPAAPHAHTA |
| | | | | FMSPHSGIGYSVFSHGNLNRALDMKS |
| | | | | PPPLNVGTENLRADGANQAMGATRA |
| | | | | NHNSARGLLYQGLHSSSSMVAMGKA |
| | | | | GLLSHSLGGYGLPSPGASEYSQPGFYH |
| | | | | SVSLQRGTVNPWQAAQPPQEPHGPHI |
| | | | | NPVESSGGCSFPSQSCTPTSPHLPSLNL |
| | | | | SIKSERSSPEHMASPTSPPLHHLRQHSP |
| | | | | MSNPDSARHTPPDTHPANGTKEFAKT |
| | | | | SYPQDQEEGGQSLRQLEMSDGWQR |
| | | | | (SEQ ID NO: 30) |

PITX1

| | | | | |
|---|---|---|---|---|
| PITX1 | Gallus | NP_001161156.2, | NM_001167684.2 | MDSFKGGMNLERLPESLRPQPSHDMA |
| | gallus | variant 1 | (encodes | SSFHLQRSSEPRDPIENSASESSDTEVP |
| | (chicken) | (SEQ ID | NP_001161156.2) | EKERSGEQKNEDGAADDPAKKKKQR |
| | | NO: 31) | | RQRTHFTSQQLQELEATFQRNRYPDM |
| | | | | SMREEIAVWTNLTEPRVRVWFKNRR |
| | | | | AKWRKRERNQQMDLCKNGYVPQFS |
| | | | | GLMQPYDDMYAGYPYNNWATKSLT |
| | | | | PAPLSTKSFTFFNSMSPLSSQSMFSAPS |
| | | | | SISSMNMPSGMGHSAVPGMANSGLN |
| | | | | NINNISGSSLNSAMSSPACPYGPPGSP |
| | | | | YSVYRDTCNSSLASLRLKSKQHSSFG |
| | | | | YSSLQSPGSSLNACQYNS (SEQ ID NO: |
| | | | | 31) |
| PITX1 | Anas | XP_038025156.1 | XM_038169228.1 | MGRKKIQISRILDQRNRQVTFTKRKFG |
| | platyrhynchos | (SEQ ID | (encodes | LMKKAYELSVLCDCEIALIIFNSTNRL |
| | (Duck) | NO: 32) | XP_038025156) | FQYASTDMDKVLLKYTEYSEPHESRT |
| | | | | NSDILETLKRKGLGLDSHELELDEGLD |
| | | | | GGEKMRKLNEGMDLTVARPRFYSPV |
| | | | | PLPEASYSSSPPAGGDGALGSTSSSPQ |
| | | | | SQGRPPAFKPSAPKLSGRSPGPMPPGI |
| | | | | GYPLFPPGSLNRALATKTPPPLYLGAD |
| | | | | GRRGEAHGSLASGRSGGSAARPLYPG |
| | | | | LQTLSPVLPPGSAGIPNHSLSGFPFLAP |
| | | | | AQAEYGAGEAPPPPGFLQPGPPASWQ |
| | | | | PPRDMAALGTSTRIVPAEDAAPGSSPQ |
| | | | | HHAISIKSERVSPGLGCPSGTPQPPPGG |
| | | | | LTSLNEAPRGSGDLQPRDDYAKGYPY |
| | | | | PLGPPRPLAEEQRATTVPVPPRRAQAV |
| | | | | DGWQR (SEQ ID NO: 32) |
| PITX1 | Meleagris | XP_010723330.1 | XM_010725028.3 | MGRKKIQISRILDQRNRQVTFTKRKFG |
| | gallopavo | (SEQ ID | (encodes | LMKKAYELSVLCDCEIALIIFNSTNRL |
| | (Turkey) | NO: 33) | XP_010723330) | FQYASTDMDKVLLKYTEYSEPHESRT |
| | | | | NSDILETLKRKGLGLESHELELEEGLD |
| | | | | PGEKLRQLNEGMDLTVARPRFYSPVP |
| | | | | LPEVPYGSSPPSSSDGALGSASSSPQSQ |
| | | | | GRPPTFKPTAPKPSGRSPGPMPPGIGY |
| | | | | PLFPAGSLNRALATKTPPPLYLGADG |
| | | | | QRRSGSGAARPLYSGLQTLSPVLASGS |
| | | | | AGIPSHSLTGFPFLTPAQAEFGAGEAP |
| | | | | PPPGFLQPGPPAPWQPPRDMAALGAS |
| | | | | SRIIPTEDLAPGSSPQPHTISIKSERVSP |
| | | | | GLGCPSGTPQPSPGSLTSLSEAPRGAA |
| | | | | DLQPRDDYTKGYPYPLPPPRPLAEEQ |
| | | | | RATATIPVPARRVQAEDAWQR (SEQ |
| | | | | ID NO: 33) |
| PITX1 | Sus scrofa | NP_001231299.1 | NM_001244370.1 | MDAFKGGMSLERLPEGLRPPPPPPHD |
| | (Porcine) | (SEQ ID | (encodes | MGPAFHLARAADPREPLENSASESSD |
| | | NO: 34) | NP_001231299.1) | TELPEKERSGEPKGPEDSSAGGAGCG |
| | | | | GAEDPAKKKKQRRQRTHFTSQQLQEL |
| | | | | EATFQRNRYPDMSMREEIAVWTNLTE |
| | | | | PRVRVWFKNRRAKWRKRERNQQLDL |
| | | | | CKGGYVPQFSGLVQPYEDVYAAGYS |
| | | | | YNNWAAKSLAPAPLSTKSFTFFNSMS |
| | | | | PLSSQSMFSAPSSISSMTMPSSMGPGA |
| | | | | VPGMPNSGLNNINNLTGSSLNSAMSP |
| | | | | GACPYGTPASPYSVYRDTCNSSLASL |
| | | | | RLKSKQHSSFGYGGLQGPASGLNACQ |
| | | | | YNS (SEQ ID NO: 34) |
| PITX1 | Bos taurus | NP_001192229.1 | NM_001205300.2 | MDAFKGGMSLERLPEGLRPPPPQPHD |
| | (Bovine) | (SEQ ID | (encodes | MGPAFHLARTADPREPLENSASESSDT |
| | | NO: 35) | NP_001192229.1) | ELPEKERGGEPKGPEDSGAGGAGCGG |
| | | | | AEDPAKKKKQRRQRTHFTSQQLQELE |
| | | | | ATFQRNRYPDMSMREEIAVWTNLTEP |
| | | | | RVRVWFKNRRAKWRKRERNQQLDL |
| | | | | CKGGYVPQFSGLVQPYDDVYAAGYS |
| | | | | YNNWAAKSLAPAPLSTKSFTFFNSMS |
| | | | | PLSSQSMFSAPSSISSMTMPSSMGPGA |
| | | | | VPGMPNSGLNNINNLTGSSLNSAMSP |
| | | | | GACPYGTPASPYSVYRDTCNSSLASL |
| | | | | RLKSKQHSSFGYGGLQGPASGLNACQ |
| | | | | YNS (SEQ ID NO: 35) |
| PITX1 | Oreochromis | XP_005456203.1 | XM_005456146.4 | MELNLSKNVISCGESEKERNAEHRSD |
| | niloticus | (SEQ ID | (encodes | DGNADDPKKKKQRRQRTHFTSQQLQ |
| | (Tilapia) | NO: 36) | XP_005456203.1) | ELEATFQRNRYPDMSTREEIAVWTNL |
| | | | | TEARVRVWFKNRRAKWRKRERNQQ |
| | | | | MDLCKNSYLPQFSGLMQPYDDMYPA |
| | | | | YTYNNWTNKGLTPAPLSTKNFTFFNS |
| | | | | MSPLTSQSMFSAPNSISSMTMASGMG |
| | | | | HSAVPGMPTPGLNNISNLNGIGTSGIN |
| | | | | SAMSSSACPYGPPGSPYSVYRDTCNSS |
| | | | | LASLRLKSKQHPTFGYSGLQSPGSSLN |
| | | | | ACQYNS (SEQ ID NO: 36) |

TERT

| | | | | |
|---|---|---|---|---|
| TERT | Gallus | NP_001026178.1 | NM_001031007.1 | MERGAQPGVGVRRLRNVAREEPFAA |
| | gallus | (SEQ ID | (encodes | VLGALRGCYAEATPLEAFVRRLQEGG |
| | (chicken) | NO: 37) | NP 001026178.1) | TGEVEVLRGDDAQCYRTFVSQCVVC |
| | | | | VPRGARAIPRPICFQQLSSQSEVITRIV |
| | | | | QRLCEKKKKNILAYGYSLLDENSCHF |
| | | | | RVLPSSCIYSYLSNTVTETIRISGLWEIL |
| | | | | LSRIGDDVMMYLLEHCALFMLVPPSN |
| | | | | CYQVCGQPIYELISRNVGPSPGFVRRR |
| | | | | YSRFKHNSLLDYVRKRLVFHRHYLSK |
| | | | | SQWWKCRPRRRGRVSSRRKRRSHRIQ |
| | | | | SLRSGYQPSAKVNFQAGRQISTVTAR |
| | | | | LEKQSCSSLCLPARAPSLKRKRDGEQ |
| | | | | VEITAKRVKVMEKEIEEQACSIVPDVN |
| | | | | QSSSQRHGTSWHVAPRAVGLIKEHYI |
| | | | | SERSNSEMSGPSVVRRSHPGKRPVAD |
| | | | | KSSFPQGVQGNKRIKTGAEKRAESNR |
| | | | | RGIEMYINPIHKPNRRGIERRINPTHKP |
| | | | | ELNSVQTEPMEGASSGDRKQENPPAH |
| | | | | LAKQLPNTLSRSTVYFEKKFLLYSRSY |
| | | | | QEYFPKSFILSRLQGCQAGGRRLIETIF |
| | | | | LSQNPLKEQQNQSLPQQKWRKKRLP |
| | | | | KRYWQMREIFQKLVKNHEKCPYLVF |
| | | | | LRKNCPVLLSEACLKKTELTLQAALP |
| | | | | GEAKVHKHTEHGKESTEGTAPNSFLA |
| | | | | PPSVLACGQPERGEQHPAEGSDPLLRE |
| | | | | LLRQHSSHWQVYGFVRECLERVIPAE |
| | | | | LWGSSHNKCRFFKNVKAFISMGKYA |
| | | | | KLSLQQLMWKMRVNDCVWLRLAKG |
| | | | | NHSVPAYEHCYREEILAKFLYWLMDS |
| | | | | YVIELLKSFFYITETMFQKNMLFYYRK |
| | | | | FIWGKLQNIGIRDHFAKVHLRALSSEE |
| | | | | MEVIRQKKYFPIASRLRFIPKMNGLRP |
| | | | | VVRLSRVVEGQKLSKESREKKIQRYN |
| | | | | TQLKNLFSVLNYERTVNTSIIGSSVFG |
| | | | | RDDIYRKWKEFVTKVFESGGEMPHFY |
| | | | | FVKGDVSRAFDTIPHKKLVEVISQVLK |
| | | | | PESQTVYGIRWYAVIMITPTGKARKL |
| | | | | YKRHVSTFEDFIPDMKQFVSKLQERTS |
| | | | | LRNAIVVEQCLTFNENSSTLFTFFLQM |
| | | | | LHNNILEIGHRYYIQCSGIPQGSILSTLL |
| | | | | CSLCYGDMENKLLCGIQKDGVLIRLID |
| | | | | DFLLVTPHLMQARTFLRTIAAGIPEYG |
| | | | | FLINAKKTVVNFPVDDIPGCSKFKHLP |
| | | | | DCRLISWCGLLLDVQTLEVYCDYSSY |
| | | | | AFTSIRSSLSFNSSRIAGKNMKCKLTA |
| | | | | VLKLKCHPLLLDLKINSLQTVLINIYKI |
| | | | | FLLQAYRFHACVLQLPFNQKVRNNPD |
| | | | | FFLRIISDTASCCYFILKAKNPGVSLGS |
| | | | | KDASGMFPFEAAEWLCYHAFIVKLSN |
| | | | | HKVIYKCLLKPLKVYKMHLFGKIPRD |
| | | | | TMELLKTVTEPSLCQDFKTILD (SEQ |
| | | | | ID NO: 37) |
| TERT | Anas | XP_038030978.1 | XM_038175050.1 | MEEGRREGEGRGAAAAPGAAAAAAG |
| | platyrhynchos | (SEQ ID | (encodes | DGGAGGTGSTGGTGGGRPHRNMAGA |
| | (Duck) | NO: 38) | XP_038030978.1) | EPFGAVLGALRGCYAQVAPLETFVRG |
| | | | | LGESGAEEAEVVRDSDAASYRTFVSQ |
| | | | | CVVCVPHGARDIPRPFSLEQLSSQSEVI |
| | | | | SRVMQRLCGKKKKNILTYGYSLLDEN |
| | | | | SSHFQIMPLSNVYSYLPNTATETMRIS |
| | | | | GLWETLLSRIGDDVMMYLLEHCAIFM |
| | | | | LVPPSNCYQVCGQPIYELISQNVESAP |
| | | | | EFVKQRLSKHKRSSLLKYMQKRLTFH |
| | | | | RQYLSKSRQSKRRQRLEANVSSVRNK |
| | | | | TSNNIQSLGSAALEKQSSSNAGLSATA |
| | | | | PSLKRKLAREQLEVTAKRARLEEKER |
| | | | | EEQACNTAPNVNQSIPKRYGTGCVAS |
| | | | | RSVSLTKEKNISQRSNSDMSRPSLVHN |
| | | | | SHRGKKSVADKSSFLQGAESNRHLKP |
| | | | | SIEMQAGSSRKGVETRRPIPRLDWVPI |
| | | | | EPAESSSSGHKKQEGPLAHLAEEVPN |
| | | | | RVLPSTIYIDRKFLYSRRYWGERFPKS |
| | | | | FLLNRLKGSQAGVKRLIETIFLSQNPF |
| | | | | GQKCNQGLPQKKRRKKKLPKRFWRM |
| | | | | RSIFQQLLKNHGKFPYVAFLRQNCPLR |
| | | | | ISDTILGKAKLLSRAPLPGQAEARKQA |
| | | | | EQLGKEPAERVASSRCESGHTNVPSS |
| | | | | VRAPLAASACGEPGGEEQIPAEASDSV |
| | | | | LRELLKEHCSHFQVYLFVRECVERVIP |
| | | | | TELWGSNHNKRRFFKNVKAFISMGK |
| | | | | YAKLSLQVLMWKMRVNDCMWLRLA |
| | | | | KGNHFVPASEHLYREEILAKFLYWLM |
| | | | | DTYVVQLLRSFFYVTETMFQKNMLFY |
| | | | | YRKCIWGKLQDIGIRKHFSKVKLRPLT |
| | | | | AEEMEAIHQKKYLPMASKLRFIPKVT |
| | | | | GLRPIVRMSGVVEAQTLSKESRAKKM |
| | | | | NHYNMQLKNLFSVLNYERTINTSYIG |
| | | | | SSVFGRDDIYKSWKTFVKKVLKSDGE |
| | | | | IPHFYYVKADVSRAFDSIPHNKLVEVI |
| | | | | SQVLKPEKKTVYCIRRYAVVMITGSG |
| | | | | KTRKLYKRHVSTFKDFMPDMKQFVS |
| | | | | RLHESTSLRDAIIVEQSLTFNETSASLF |
| | | | | NFFLQMLNNNILEIERSYYLQCSGIPQ |
| | | | | GSLLSTLLCSLCYGDMENKLFSGVQK |
| | | | | DGVLIRLIDDFLLVTPHLTHARTFLRT |
| | | | | LAMGIPEYGFLINPKKTVVNFSADDIP |
| | | | | ECSEFKQLPNCRLIPWCGLLLDTQTLE |
| | | | | VYCDYSSYSCTSIRSSLSFNSNRTAGK |
| | | | | NMKHKLLAVLKLKCHGLFLDLQINSL |
| | | | | KTVFINVYKIFLLQAYRFHACVIQLPF |
| | | | | NQKVRNNPDFFLRVIAENASCCYSML |
| | | | | KAKNPGFTLGNRGASGMFPSEAAEW |
| | | | | LCYHAFTVKLSNHKVVYKCLLKPLKF |
| | | | | CMMQLFRKIPKDTKALLKTVTEPSICK |
| | | | | DFKSILD (SEQ ID NO: 38) |
| TERT | Meleagris | XP_031408581.1 | XM_031552721.1 | MEASRNIEFSGGAACASAFWGTVTW |
| | gallopavo | (SEQ ID | (encodes | RPGVGSRGGEERHRVLPGHEEQDQK |
| | (Turkey) | NO: 39) | XP_031408581.1) | GSTEQASVRRNVRLKSRPGCLAAVSA |
| | | | | SLGHVLFVEKLLIKKSVLPFSVFVQLS |
| | | | | SQSEVITRIVQRLCEKKKKNILAYGYS |
| | | | | LLDENSCHFRILPSSCIYSYLPNTVTETI |
| | | | | RISGLWEILLSRIGDDVMMYLLEHCA |
| | | | | LFMLVPPSNCYQVCGQPIYELISRNIGP |
| | | | | SPGFVRRRYSRFKHNNLLNYVRKRLV |
| | | | | FHRHYLSKSQWWKCGPRRQGRVSSR |
| | | | | RKRRTHRIQSPRSGYQSSAKVNFQAG |
| | | | | MRISTVTAHLEKQNCSSLCLPARTPSL |
| | | | | KRKRDGEQVETTAKRVKVMEREEQA |
| | | | | CSIVPDVNRSSSRRHGVWHVAPRAVG |
| | | | | LIKERYVSERSYSEMSGPSVVHRSHPG |
| | | | | KRPVADKSSFPRGVQGNKHIKTGAEK |
| | | | | RAESNKRGIEMYINPICKPNRRGIERHI |
| | | | | NPTHKPGLNSVQTEPMESASSGDRKQ |
| | | | | ENPPAHLAKQLPNTFLRSAVYFEKKF |
| | | | | LLYSRSYQEYFPKSFILSRLQGCQAGG |
| | | | | RQLIETIFLSQNPLKEKQNQSLKQQKW |
| | | | | RKKRLPKRYWQMREIFQKLLKNHEK |
| | | | | CPYLVFLRKNCPVLLSEACLKKTELTL |
| | | | | QAALPGEAKVHKHTEHGEETTEGTAP |
| | | | | NSFYTPPSMPLCGQTEREEQHLAEGS |
| | | | | DPLLRELLRQHSSHWQVYGFVRECLE |
| | | | | RVIPAELWGSSHNKCRFFKNVKAFIS |
| | | | | MGKYAKLSLQQLMWKMRVNDCVW |
| | | | | LRLAKGNHSVPAYEHCYREEILAKFL |
| | | | | YWLMDSYVIELLKSFFYITETMFQKN |
| | | | | MLFYYRKFIWGKLQNIGIRNHFAKVH |
| | | | | LRALSSEEMEVIHQKKYFPIASRLRFIP |
| | | | | KINGLRPVVRLSRVVEGQKLSKESRE |
| | | | | KKIQRYNTQLKNLFSVLNYERTVNTSI |
| | | | | IGSSVFGRDDIYRKWKEFVTKVFESG |
| | | | | GEMPHFYFVKGDVSRAFDTIPHKKLV |
| | | | | EVISQVLKPESQTVYGIRWYAVIMITP |
| | | | | TGKARKLYKRHVSTFEDFIPDMKQFV |
| | | | | SKLQERTSLRNAIVVEQCLTFNENSST |
| | | | | LFTFFLQMLHNNILEIGHRYYIQCSGIP |
| | | | | QGSILSTLLCSLCYGDMENKLLCGIQK |
| | | | | DGILIRLIDDFLLVTPHLMQAKTFLRTI |
| | | | | AAGIPEYGFLINAKKTVVNFPVDDIPG |
| | | | | CSKFKQLPDCRLISWCGLLLDMQTLE |
| | | | | VYCDYSSYAFTSIRSSLSFNSSRIAGKN |
| | | | | MKCKLTAVLKLKCHPLFLDLKINSLK |
| | | | | TVLINIYKIFLLQAYRFHACVLQLPFN |
| | | | | QKVRNNPYFFVRIISDTASCCYFILKA |
| | | | | KNPGVCLGCKDASGMFPFEAAEWLC |
| | | | | YHAFIVKLSNHKVIYKCLLKPLKVYK |
| | | | | MHLFGKIPRDTMVLLKTVTEPSLCQD |
| | | | | FKTILD (SEQ ID NO: 39) |
| TERT | Sus scrofa | NP_001231229.2 | NM_001244300.2 | MPRAPRCRAVRSLLRDRYRQVLPLAT |
| | (Porcine) | (SEQ ID | (encodes | FVRRLGPEGRRLVRRGDPAAYRALVA |
| | | NO: 40) | NP_001231229.2) | QCLVCVPWDAQPPPASPSFRQVSCLK |
| | | | | ELVARVVQRLCERGARNVLAFGFALL |
| | | | | DGARGGPPVAFTTSVRSYLPNTVTDT |
| | | | | LRGSGAWGLLLRRVGDDVLTHLLAR |
| | | | | CALYLLVPPSCAYQVCGPPLYDLYTA |
| | | | | AEARPMRHKGQTPTGLGLTRPVCNGE |
| | | | | AGRPQEQRAQGVRRRRGRAGGHPLP |
| | | | | AKRPRHVPEPEQGPEGQASRAHQGRA |
| | | | | PGPSDSDPPVMTPTRAAAKAKSREGE |
| | | | | APGTRHLSPQAGGARGTCPPSWWQP |
| | | | | HLQGKPSPHVCAAETKRFLYCSGSKE |
| | | | | GLRRSFLLCSLPPSLAGAGRLVEVIFL |
| | | | | ASKPGQPGARRVPARYWRMRPLFRE |
| | | | | LLKNHARCPYKALLRAHCPLRAAATL |
| | | | | SGSGGQVCDHKVGPLAPERLAAAAE |
| | | | | GDSASRRLVQLLRQHSSPWQVYRLLR |
| | | | | ACLHRLVPPGLWGSPHNKRRFLKNV |
| | | | | KKLVSLGKHARLSLQELMWKMKVQ |
| | | | | DCIWLRRSPDARHVQAAEHRLREAIL |
| | | | | AKFLRWLMGTYVVELLRSFFYVTETT |
| | | | | FQKNRLFFFRKRIWSRLQSAGIRQHLD |
| | | | | RVRLRELSEAEIRRRREARPAVLTSKL |
| | | | | RFVPKPDGLRPIVNMANVVRARTGPG |
| | | | | DKKVRRLTGQVKTLFAVLNYERARR |
| | | | | PRLLGASVLGVGDIHRAWRAFVLPLR |
| | | | | AQDPAPPLYFVKVDVTGAYDALPQD |
| | | | | RLLEVVANVIRPHESTYCVRQCAVLR |
| | | | | RTARGHVRKSFQTHVSTFADLQPYMR |
| | | | | QFVAHLQATGPLRDAVVIEQSCSLNE |
| | | | | AGSRLLELFLSLLRNHVIRIGGRSYVQ |
| | | | | CQGIPQGSILSTLLCSLCYGDMENRLF |
| | | | | PGIQRDGVLLRLVDDFLLVTPHLTRA |
| | | | | KAFLRTLVRGVPEYGCLANLRKTAVN |
| | | | | FPVEDGARGGPAPLQLPAHCLFPWCG |
| | | | | LLLDTRTLEVHCDYASYARTSIRASLT |
| | | | | FNQGFKPGRNMRRKLLAVLRLKCHGI |
| | | | | LLDLQVNSLPTVLANVYKIFLLQAYR |
| | | | | FHACVLQLPFRQPLARNPSFFLRLVSD |
| | | | | TASCCYSLLKARNAGMSLGARGASGP |
| | | | | FPSEAAEWLCLHAFLLKLVRHRVTYS |
| | | | | CLLGPLRAARERLCQRLPGATLAALE |
| | | | | AAADPALTTDFRTILD (SEQ ID NO: |
| | | | | 40) |
| TERT | Bos taurus | NP_001039707.1 | NM_001046242.1 | MPRAPRCRAVRALLRASYRQVLPLAA |
| | (Bovine) | (SEQ ID | (encodes | FVRRLRPQGHRLVRRGDPAAFRALVA |
| | | NO: 41) | NP_001231229.2) | QCLVCVPWDAQPPPAAPSFRQVSCLK |
| | | | | ELVARVVQRLCERGARNVLAFGFTLL |
| | | | | AGARGGPPVAFTTSVRSYLPNTVTDT |
| | | | | LRGSGAWGLLLHRVGDDVLTHLLSR |
| | | | | CALYLLVPPTCAYQVCGPPLYDLRAA |
| | | | | AAAARRPTRQVGGTRAGFGLPRPASS |
| | | | | NGGHGEAEGLLEARAQGARRRRSSA |
| | | | | RGRLPPAKRPRRGLEPGRDLEGQVAR |
| | | | | SPPRVVTPTRDAAEAKSRKGDVPGPC |
| | | | | RLFPGGERGVGSASWRLSPSEGEPGA |
| | | | | GACAETKRFLYCSGGGEQLRRSFLLC |
| | | | | SLPPSLAGARTLVETIFLDSKPGPPGAP |
| | | | | RRPRRLPARYWQMRPLFRKLLGNHA |
| | | | | RSPYGALLRAHCPLPASAPRAGPDHQ |
| | | | | KCPGVGGCPSERPAAAPEGEANSGRL |
| | | | | VQLLRQHSSPWQVYGLLRACLRRLVP |
| | | | | AGLWGSRHNERRFLRNVKKLLSLGK |
| | | | | HGRLSQQELTWKMKVQDCAWLRASP |
| | | | | GARCVPAAEHRQREAVLGRFLHWLM |
| | | | | GAYVVELLRSFFYVTETTFQKNRLFFF |
| | | | | RKRIWSQLQRLGVRQHLDRVRLRELS |
| | | | | EAEVRQHQEARPALLTSRLRFVPKPG |
| | | | | GLRPIVNVGCVEGAPAPPRDKKVQHL |
| | | | | SSRVKTLFAVLNYERARRPGLLGASV |
| | | | | LGMDDIHRAWRAFVLPLRARGPAPPL |
| | | | | YFVKVDVVGAYDALPQDKLAEVIAN |
| | | | | VLQPQENTYCVRHCAMVRTARGRMR |
| | | | | KSFKRHVSTFSDFQPYLRQLVEHLQA |
| | | | | MGSLRDAVVIEQSCSLNEPGSSLFNLF |
| | | | | LHLVRSHVIRIGGRSYIQCQGIPQGSIL |
| | | | | STLLCSFCYGDMENKLFPGVQQDGVL |
| | | | | LRLVDDFLLVTPHLTRARDFLRTLVR |
| | | | | GVPEYGCQVNLRKTVVNFPVEPGALG |
| | | | | GAAPLQLPAHCLFPWCGLLLDTRTLE |
| | | | | VHGDHSSYARTSIRASLTFTQGFKPGR |
| | | | | NMRRKLLAVLQLKCHGLFLDLQVNS |
| | | | | LQTVFTNVYKIFLLQAYRFHACVLQL |
| | | | | PFSQPVRSSPAFFLQVIADTASRGYAL |
| | | | | LKARNAGASLGARGAAGLFPSEAAQ |
| | | | | WLCLHAFLLKLARHRVTYSRLLGALR |
| | | | | TARARLHRQLPGPTRAALEAAADPAL |
| | | | | TADFKTILD (SEQ ID NO: 41) |
| TERT | Oreochromis | XP_025758119.1 | XM_025902334.1 | MKICSLPELLAFVLNTLKRKRKRNVL |
| | niloticus | (SEQ ID | (encodes | AHGYNFQTLAQEDRDADFLKFQGDV |
| | (Tilapia) | NO: 42) | XP_025758119.1) | TQSAAYIHGSDLWKKVTMRLGTDITQ |
| | | | | YLLESCSVFVAVPPSCVFQVCGPPVY |
| | | | | DRVSMTMASSGFFLQPGVRKHNRTKI |
| | | | | ESCRGSVSLKQKRTVVNPAASKKMK |
| | | | | RRNKGGKKGKRKRETGEEEEVAVCS |
| | | | | RKRRRVASIEHQQAIQPVGSEKEGQV |
| | | | | VPVESAPPAAFKQPVEMPTLEGGPSW |
| | | | | RSGIFPPLPPSQCFIRTLGFLYGGRGMR |
| | | | | GFLLNRRKKTAHGSRRLQGQDLVRIV |
| | | | | FFEGLAYLNGVERKPKKLPQRFFGMV |
| | | | | PLFRQLLQQHRSCSYTKILQRLCPSIEE |
| | | | | SNAGQGELNSLLPQHCAPHRVYLFVR |
| | | | | ECLSSVIPQELWGSDQNRLHFFARVRT |
| | | | | FLRSGKFERLSLAELMWKIKVNDCD |
| | | | | WLKRSKTGCFPPSELAYRTQVLGQFL |
| | | | | AWLLDGYVTGLVRACFYATESIGQK |
| | | | | NAIRFYRQEVWAKLQDLAFRGHLSK |
| | | | | GQMEELTPAQVASLPKGTVISRLRFIP |
| | | | | KTDGMRPITRVIGADAKTRLYRGRVR |
| | | | | DLLDMLRACVRATPSLLGSTVWGMT |
| | | | | DIHKVLCSLAPAQKEKPQPLYFVKVD |
| | | | | VSGAYESLPHDKLIEVIGQALSPVHDE |
| | | | | LFTIRRYAKIWADSHEGLKKAFVRQA |
| | | | | DFLEDNMGSTNMKGFLTSLQRKGKV |
| | | | | HHAILVEQHFCSDLHGREALQFFTQM |
| | | | | LTGSVVQYGKKTYRQCRGIPQGSVVS |
| | | | | SLLCCLCYGHMENLLFKDIPGHKGCL |
| | | | | MRLVDDFLLITPDQHEAQAFLKILLAG |
| | | | | VPQYGLAVNPQKVVLNFQVSGSVAS |
| | | | | CPDIRILPPHCLFPWCGLLLDTHKLDV |
| | | | | YKDYSSYAGLSLRYSLTLGSSHSAGQ |
| | | | | QMKRKLMAILRLKCHALFFDLKTNSL |
| | | | | EAVYKNIYKLVLLHACRFHVCAQSLP |
| | | | | FGQTVSKNPVFFLQLIWEMAQYCNKL |
| | | | | IRRSNKGLILGDKAQTGIVQYEAVELL |
| | | | | FCLCFLLVLSQHRLLYKDLLAHLHKR |
| | | | | KRSLERRLGDLRLARVRQAASPRTPV |
| | | | | DFLAIQT (SEQ ID NO: 42) |

### 6.4. Inducing Myogenic-Specific Differentiation

This disclosure also provides methods for differentiating a cell line or an immortalized cell line having improved myogenic differentiation capacity into a cell type of interest (e.g., a myoblast). In some embodiments, a cell line or an immortalized cell line having improved myogenic differentiation capacity is differentiated into a cell of type of interest (e.g., a myoblast) using a differentiation media. In some embodiments, a differentiation media comprises base media without any additional additives. Non-limiting examples of base media include: DMEM/F-12, MEM, IMDM, and DMEM. In some embodiments, a differentiation media comprises base media including serum (e.g., horse serum, bovine serum, chicken serum, or a combination thereof). For example, differentiation media includes about 0.5% serum, about 1.0% serum, about 2.0% serum, about 3.0% serum, about 4% serum, about 5% serum, about 6% serum, about 7% serum, about 8% serum, about 9% serum, or about 10% serum. In some embodiments, differentiation media includes about 2% serum (e.g., horse serum, bovine serum, chicken serum, or a combination thereof).

In some embodiments, a cell line or an immortalized cell line having improved myogenic differentiation capacity (e.g., as a result of the methods described herein) is exposed to the differentiation media for a period of time. In some embodiments, the period of time is any amount of time needed for the cell line or the immortalized cell line to differentiate to a cell type of interest. Non-limiting examples of a period of time include: about 1 day, about 2 days, about 3 days, about 4 days, about 5 days, about 6 days, about 7 days, about 8 days, about 9 days, about 10 days, about 11 days, about 12 days, about 13 days, or about 14 days. In some embodiments, a cell line or an immortalized cell line having improved myogenic differentiation capacity (e.g., as a result of the methods described herein) is allowed to reach at least 80% (e.g., at least 85%, at least 90%, or at least 95%) confluency before being exposed to differentiation media.

Also provided herein is an in vitro method for producing a cell-based meat product, comprising: (a) exposing (contacting) the cell line or the immortalized cell line to (with) at least a first Activin A inhibitor and at least a first BMP inhibitor; (b) transforming, introducing, or incorporating into the genome of, the cell at least a first myogenic regulatory factor polypeptide (e.g., any of the myogenic regulatory factor polypeptides described herein), thereby producing a recombinant cell line expressing the one or more myogenic regulatory factors; and (c) inducing myogenic specific differentiation. In some embodiments, the in vitro method for producing a cell based meat product includes: (a) exposing (contacting) the cell line or the immortalized cell line to (with) at least one Activin A inhibitor, at least one BMP inhibitor, and at least one WNT activator; (b) transforming, introducing, or incorporating into the genome of, the cell (e.g., the cell of the immortalized cell line) with at least a first myogenic regulatory factor polypeptide (e.g., any of the myogenic regulatory factor polypeptides described herein) producing a recombinant cell line expressing the one or more myogenic regulatory factors; and (c) inducing myogenic specific differentiation.

Non-limiting examples of myogenic differentiation are described in WO2019014652A1 and WO2015066377A1, both of which are herein incorporated by reference in their entireties.

### 6.5. Nucleic Acids/Vectors

Also provided herein are nucleic acid sequences that encode any of the myogenic regulatory factors described herein (e.g., any of the myogenic factors, fragments or variants thereof).

Also provided herein is a nucleic acid construct (i.e., a vector) that includes any of the nucleic acid sequences encoding any of the myogenic regulatory factors described herein. Any of the vectors described herein can be an expression vector. For example, an expression vector can include a promoter sequence operably linked to a first sequence encoding any of the myogenic regulatory factors described herein. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some cases, a vector can include sufficient cis-acting elements that supplement expression where the remaining elements needed for expression can be supplied by the host cell.

In some embodiments, a vector includes a nucleic acid sequence encoding a single myogenic regulatory factor of fragment thereof. In some embodiments, a vector includes nucleic acid sequences encoding two or more, three or more, or four or more myogenic regulatory factors. In such cases, each of the two or more nucleic acid sequences are operably linked to a promoter sequence or another nucleic acid sequence via a self-cleaving polypeptide or IRES. As used herein, the term “operably linked” is well known in the art and refers to genetic components that are combined such that they carry out their normal functions. For example, a nucleic acid sequence is operably linked to a promoter when its transcription is under the control of the promoter. In another example, a nucleic acid sequence can be operably linked to other nucleic acid sequences by a self-cleaving 2A polypeptide or an internal ribosome entry site (IRES). In such cases, the self-cleaving 2A polypeptide allows the second nucleic acid sequence to be under the control of the promoter operably linked to the first nucleic acid sequence. In some cases, the nucleic acid sequences described herein can be operably linked to any other nucleic acid sequence described herein using a self-cleaving 2A polypeptide or IRES. In some cases, the nucleic acid sequences are all included on one vector and operably linked either to a promoter upstream of the nucleic acid sequences or operably linked to the other nucleic acid sequences through a self-cleaving 2A polypeptide or an IRES.

In some embodiments, a single nucleic acid construct encodes MYOD or a fragment thereof. In some embodiments, the single nucleic acid construct encoding MyoD or a fragment thereof comprises a sequence of SEQ ID NO: 43. In some embodiments, the single nucleic acid construct encoding MYOD or a fragment thereof, comprises a sequence that is at least 80% (e.g., at least 85%, 90, 95%, 96%, 97%, 98%, or 99%) identical to SEQ ID NO: 43. In some embodiments, the single nucleic acid construct encoding MYOD or a fragment thereof comprises a sequence of SEQ ID NO: 44 or 46. In some embodiments, the single nucleic acid construct encoding MYOD or a fragment thereof, comprises a sequence that is at least 80% (e.g., at least 85%, 90, 95%, 96%, 97%, 98%, 99% or 100%) identical to nucleotides 2886-4511, or a fragment thereof, of SEQ ID NO: 44. In some embodiments, the single nucleic acid construct encoding MYOD or a fragment thereof, comprises a sequence that is at least 80% (e.g., at least 85%, 90, 95%, 96%, 97%, 98%, 99% or 100%) identical to nucleotides 3750-6192, or a fragment thereof, of SEQ ID NO: 46.

In some embodiments, a single nucleic acid construct encodes MYOD or a fragment thereof, PAX7 or a fragment thereof, and MEF2B or a fragment thereof, and includes self-cleaving 2A polypeptides to operably link the coding sequences. In some embodiments, the single nucleic acid construct encoding MYOD or a fragment thereof, PAX7 or a fragment thereof, and MEF2B or a fragment thereof, comprises a sequence of SEQ ID NO: 45. In some embodiments, the single nucleic acid construct encoding MyoD or a fragment thereof, Pax7 or a fragment thereof, and MEF2b or a fragment thereof, comprises a sequence that is at least 80% (e.g., at least 85%, 90, 95%, 96%, 97%, 98%, 99% or 100%) identical to nucleotides 2108-7408, or a fragment thereof, of SEQ ID NO: 45.

In some embodiments, a single nucleic acid constructs encodes PAX3/PAX7 (or a fragment thereof), PITX1 (or a fragment thereof), and MEF2B (or a fragment thereof), and includes self-cleaving 2A polypeptides to operably link the coding sequences. In some embodiments, a single nucleic acid constructs encodes MYOD (or a fragment thereof), PAX3/PAX7 (or a fragment thereof), PITX1 (or a fragment thereof), and MEF2B (or a fragment thereof), and includes self-cleaving 2A polypeptides to operably link the coding sequences.

Also provided herein are a set of vectors that include two or more vectors. For example, the set of vectors include a first vector comprising a nucleic acid sequence encoding a MYOD polypeptide (or a fragment thereof), a second vector comprising a nucleic acid sequence encoding a PAX7 polypeptide (or a fragment thereof), and a third vector comprising a nucleic acid sequence encoding a MEF2B polypeptide (or a fragment thereof). In another example, the set of vectors include a first vector comprising a nucleic acid sequence encoding a PAX3 or a PAX7 polypeptide (or a fragment thereof), a second vector comprising a nucleic acid sequence encoding a MEF2B polypeptide (or a fragment thereof), and a third vector comprising a nucleic acid sequence encoding a PITX1 polypeptide (or a fragment thereof). In yet another example, the set of vectors include a first vector comprising a nucleic acid sequence encoding a MYOD polypeptide (or a fragment thereof), a second vector comprising a nucleic acid sequence encoding a PAX3 or a PAX7 polypeptide (or a fragment thereof), a third vector comprising a nucleic acid sequence encoding a MEF2B polypeptide (or a fragment thereof), and a fourth vector comprising a nucleic acid sequence encoding a PITX1 polypeptide (or a fragment thereof).

In some embodiments, a vector system is used to integrate a nucleic acid sequence encoding one or more myogenic regulatory factors into the genome of the cell (e.g., a cell of the immortalized cell line). In some embodiments, the vector system is a phiC31 Integrase Vector System. Additional non-limiting examples of vector systems that can be used to integrate a nucleic acid sequence encoding one or more myogenic regulatory factors into the genome of the cells (e.g., the immortalized cell line) include: a sleeping beauty transposon system (as described in U.S. Pat. No. 7,985,739), a piggyBac transposition system (as described in US20090042297), CRISPR/Cas-mediated knockin, and viral vector-mediated integration. In some embodiments, a vector is a viral vector. Non-limiting examples of viral vectors include adenovirus, adeno-associated virus, lentivirus, and retrovirus.

In some embodiments, a nucleic acid sequence that encodes any of the myogenic regulatory factors described herein is operably linked to a promoter. In some embodiments, the promoter is a muscle-specific promoter. In some embodiments, the muscle-specific promoter is selected from the group consisting of: skeletal β-action, myosin light chain 2a, dystrophin, SPc-512, muscle creatine kinase, and synthetic muscle promoters. In some embodiments, the promoter is a constitutively active promoter. In some embodiments, a promoter is selected from the group consisting of: EF1 (e.g., EF1alpha), PGK, CMV, RSV, and β-actin. In some embodiments, the promoter is a PGK promoter. In some embodiments, a vector comprises a promoter operably linked to any of the nucleic acid sequences described herein.

In some embodiments, a nucleic acid sequence that encodes any of the myogenic regulatory factors described herein are mRNA molecules. In such cases, an immortalized cell is transformed with the one or more mRNA molecules. The mRNA molecule is prepared prior to transformation using techniques known in the art.

### 6.6. Methods of Transducing Cells

Methods of introducing nucleic acids and expression vectors into a cell (e.g., an immortalized cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral, retroviral, and lentiviral transduction), lipid nanoparticle (LNP) transfection, and nanoparticle transfection.

### 6.7. Kits and Culture Media

Also provided herein are kits comprising any of the immortalized cells, any of the cells derived from the immortalized cells, any of the nucleic acid sequences encoding any of the one or more myogenic regulatory factors, any of the Activin A/TGF-β inhibitors, any of the BMP inhibitors, any of the WNT activators, or any of the HDAC inhibitors described herein. In some embodiments, the kit further comprises an immortalization agent (e.g., a nucleic acid sequence encoding a TERT polypeptide (or a fragment thereof)). In some embodiments, the kit includes instructions for performing any of the methods described herein.

Also provided herein are any of the culture medias described herein. In some embodiments, the culture media is as described herein, for example, in Section 6.2.1.

### 6.8. Cells

Also provided herein are cell line(s) capable of self-renewal for cultured food production. In some cases, the cell line(s) capable of self-renewal are immortalized cell line(s) including those generated as described herein. These cell lines are then differentiated to cell types of interest (e.g., myogenic cells).

Also provided herein are immortalized cells (e.g., any of the immortalized cells described herein). In some embodiments, the immortalized cells are fibroblasts. In some embodiments, the immortalized cells comprise any of the nucleic acids described herein that encode any of the myogenic regulatory factors described herein. In some embodiments, an immortalized cell is immortalized prior to performing the methods described herein. In some embodiments, the methods provided herein include a step of immortalizing a cell. In some embodiments, a cell is immortalized by transforming the cell with TERT.

Also provided herein are cells comprising any of the nucleic acids described herein that encode for an immortalization agent (e.g., nucleic acid sequence encoding TERT). In some embodiments, a cell includes a nucleic acid sequence that encodes an immortalization agent and a nucleic acid sequence that encodes one or more myogenic regulatory factors.

Also provided herein are cells derived from the cell, cell lines, immortalized cells, or immortalized cell lines. For example, as a result of the application of the methods described herein, a cell line or an immortalized cell line expresses one or more myogenic markers (e.g., Pax7, MyHC1, MyoG, and MyoD). Non-limiting examples of cells derived from the cell line or the immortalized cell lines include myoblasts, myotubes, multinucleated myotubes, satellite cells, skeletal muscle fibers, or any combination thereof.

In some embodiments, the cell line or immortalized cell line is from a livestock, poultry, game or aquatic animal species. In some embodiments, the cell line or immortalized cell line are from a chicken, duck, or turkey. In some embodiments, the cell line or immortalized cell line are from a fish. In some embodiments, the cell line or immortalized cell line are from a livestock species. In some embodiments, the livestock species is porcine or bovine.

In some embodiment, the cell line, late passage cell line, or immortalized cell line is derived from a species selected from Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus.

In some embodiments, the cell line or immortalized cell is isolated from Gallus gallus (chicken). In some embodiments, the cell is isolated from chicken skin. In some embodiments, the cell is isolated from chicken muscle. In some embodiments, the cell is isolated from a chicken (e.g., chicken skin or chicken muscle) and cultured until a monoculture of cells is established (e.g., a monoculture of fibroblasts originating from the isolated chicken cells). In some embodiments, a population of cells are isolated from a chicken (e.g., chicken skin or chicken muscle).

In some embodiments, the cell line, late passage cell line, or immortalized cell line is isolated from Bovine taurus (“cow” or “bovine”). In some embodiments, the cell is isolated from bovine skin. In some embodiments, the cell is isolated from bovine muscle. In some embodiments, the cell is isolated from a cow (e.g., bovine skin or bovine muscle) and cultured until a monoculture of cells is established (e.g., a monoculture of fibroblasts originating from the isolated bovine cells). In some embodiments, a population of cells are isolated from a cow (e.g., bovine skin or bovine muscle).

In some embodiments, the cell line or immortalized cell is selected from the group consisting of: a myoblast, an immortalized myoblast, an immortalized primary myoblast, a muscle satellite cell, and a muscle stem cell. In some embodiments, the immortalized cell is an immortalized myoblast or an immortalized primary myoblast.

In some embodiments, the cell line (e.g., a cell line that is ultimately immortalized) is a fibroblast. For example, the cell is an immortalized fibroblast.

In some embodiments, skeletal muscle satellite cells are isolated from a chicken. In adults these are quiescent mononucleated myogenic cells that act as a reserve population of cells, able to proliferate and/or differentiate upon stimulation and give rise to regenerated muscle and to more satellite cells.

In some embodiments, a cell line or an immortalized cell is not a stem cell (e.g., a muscle stem cell, a muscle satellite cell, or a pluripotent stem cell). In some embodiments, a cell line or an immortalized cell is not a pluripotent stem cell line (e.g., an embryonic stem cell or an induced pluripotent stem cell).

In some embodiments, a cell line or an immortalized cell comprises one or more stem cells (e.g., an adult stem cell (e.g., a mesenchymal stem cell)).

In some embodiments, a cell line is a late-passage cell line. Non-limiting examples of late passage cells include an immortalized myoblast and an immortalized fibroblast. In some embodiments, a late-passage cell includes a senescent cell. Cellular senescence can be measured using cell proliferation assays, observed changes in cellular morphology, and biomarker expression, among other techniques known in the art.

In some embodiments, a late-passage cell line refers to a cell or cell line that has been passaged (e.g., passage refers to the number of times the culture including the cell has been subcultured) at least 40 times (e.g., at least 45 times, at least 50 times, at least 60 times, at least 65 times, at least 70 times, at least 75 times, at least 80 times, at least 85 times, at least 90 times, at least 95 times, at least 100 times, at least 110 times, at least 120 times or at least 130 times).

Also provided herein are cell banks comprising cells, populations of cells, cell lines, or immortalized cell lines (e.g., immortalized fibroblast cells lines) generated according to the methods described herein.

In some embodiments, as a result of the methods provided herein, a cell bank comprises a cell, population of cells, cell line, or an immortalized cell line having increased differentiation capacity. In some embodiments, as a result of the methods provided herein, a cell bank comprises a cell, population of cells, cell line, or an immortalized cell line having increased myogenic differentiation capacity.

In some embodiments, a cell bank provides a cell, population of cells, cell line, or an immortalized cell line for use in inducing myogenic-specific differentiation. In some embodiments, a cell bank comprising a cell, population of cells, cell line, or an immortalized cell line having improved differentiation capacity is differentiated into a cell of type of interest (e.g., a myoblast, myocyte or myotube) using a differentiation media. In some embodiments, a cell bank comprising a cell, population of cells, cell line, or an immortalized cell line having improved differentiation capacity (i.e., as a result of the methods provided herein) is differentiated according to methods described in WO2019014652A1 and/or WO2015066377A1, both of which are herein incorporated by reference in their entireties.

### 6.9. Summary of Experimental Observations

Applicant evaluated the myogenic differentiation capacity of an immortalized fibroblast cell line following exposure to at least a first Activin A inhibitor, at least a first BMP inhibitor, at least a first WNT activator, or a combination thereof. Applicant demonstrated that exposing an immortalized cell line to an Activin A inhibitor, a BMP inhibitor, and a WNT activator led to improved differentiation capacity (e.g., as indicated by expression of myogenic marker Pax7) and improved differentiation to myotubes as demonstrated in part by expression of myogenic differentiation marker MyHC.

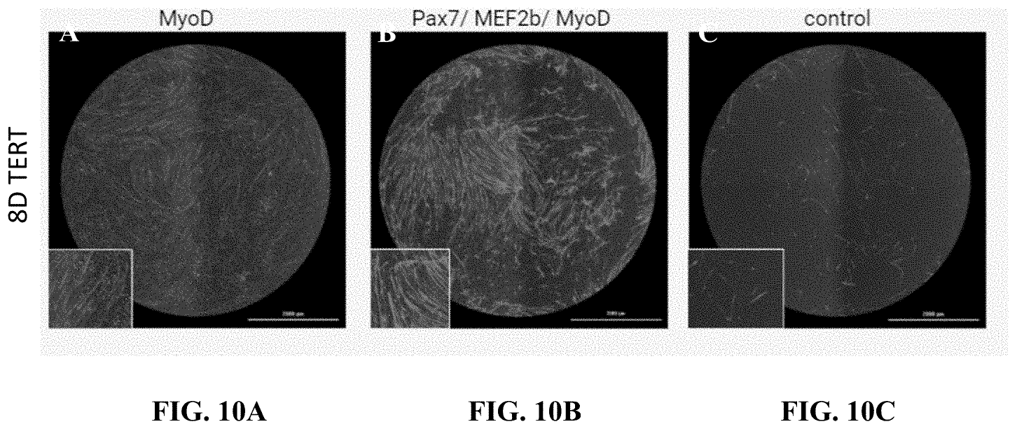

Applicant further tested differentiation capacity of an immortalized cell line following transduction with a polynucleotide encoding at least a first myogenic regulatory factor polypeptide and optionally contacting the transduced cell line with a culture media comprising an Activin A inhibitor, a BMP inhibitor, and a WNT activator (see, e.g., FIG. 10). Applicant demonstrated that transforming with MYOD alone was produce small increases in myogenic differentiation capacity (e.g., as indicated by myotube formation and myogenic marker expression) in a late passage, TERT-immortalized fibroblast cell line. In contrast, Applicant demonstrated that transduction with a polynucleotide encoding 7MM (PAX7, MYOD, and MEF2B) in combination with culture media ME9 produced robust increases in myogenic differentiation capacity (e.g., as indicated by myotube formation and myogenic marker expression) in the late-passage, TERT-immortalized fibroblast cell line.

Overall, this work demonstrated the ability to improve myogenic differentiation capacity of late passage immortalized cell lines that lost myogenic differentiation capacity following extended culture periods. As extended culture periods are required for adapting cell lines into the particular culture formats and cell culture media typically required for generating cell based meat products, this disclosure is especially powerful as it provides (1) a method for ensuring myogenic differentiation capacity is not lost despite the extended culture periods and/or (2) a method for restoring (i.e., improving) the myogenic differentiation capacity if it is reduced or lost during the extended culture periods.

### 7. EXAMPLES

### 7.1. Experimental Procedures/Methods

### 7.1.1. Gene Overexpression

Cells were transfected with plasmid(s) containing gene(s) of interest (e.g., TERT, MyoD, etc) and a plasmid containing an integrase (PhiC31) in order to incorporate the genes of interest into the genome of the cell for stable expression. The integrated plasmids included antibiotic resistance genes that allowed for antibiotic selection of the transfected population (e.g., puromycin). Alternatively, mRNA encoding the gene(s) of interest (MyoD) is transfected directly into the cells to achieve transient gene expression.

### 7.1.2. Assessment of Myogenicity

qRT-PCR (real-time quantitative reverse transcription). Messenger RNA (mRNA) was isolated from cells according to standard methods. Gene expression was assessed with primer/probe sets specifically designed to amplify genes of interest (e.g., myogenic marker, including downstream myogenic markers). mRNA was reverse transcribed to generate cDNA. quantitative PCR (qPCR) was performed on the cDNA to assess expression of myogenic factors relative to a housekeeping gene. Expression of higher levels of Myf6, MyoD, MyoG, MYMK, MyHC1e as compared to controls suggest improved myogenic differentiation capacity. Additionally, high levels of MyHC1e are indicative of cells that can mature to form myotubes.

Using immunohistochemistry. Cells were seeded in a 96-well plate at a low density (5000-10,000 cells/cm2) to allow cells to grow in the presence or absence of different small molecule combinations. After 2 days of media exposure, cells were fixed with 4% paraformaldehyde (PFA) and washed. Cells were permeabilized with 0.05% PBS-T (triton-x), blocked with normal goat serum (Millipore Sigma), and were incubated with antibodies (Developmental Studies Hybridoma Bank) specific for Pax7, a muscle stem cell marker, and subsequently with secondary antibodies (Thermofisher).

Alternatively, after 2-3 days of media exposure, media was changed to myotube differentiation medium (e.g., media comprising 2% horse serum). After 2 or more days of culturing cells in differentiation medium, cells were fixed with PFA followed by incubating fixed cells with antibodies specific for myosin heavy chain (MyHC). Nuclei of cells were counterstained with DAPI (4′,6-diamidino-2-phenylindole) (Thermofisher) to visualize and represent all cells in the imaged area. All immunostained samples were imaged by Cytation 5 (Biotek) microscopy and analyzed via Gen5 software. Pax7 was used as a proxy for myogenic differentiation capacity with increases correlating to improved myogenic differentiation capacity. In addition, increases in total percent area of MyHC indicates improved myogenic differentiation capacity.

### 7.1.3. Media Screening

A media panel was designed to include culture media comprising one or more signaling pathway agonists, antagonists, or a combination thereof. The aim was to activate or inactivate three major pathways in stem cell biology, WNT, ActivinA/TGF, BMP. CHIR99021 (5 μM), Activin A (25 ng/mL), or BMP4 (10 ng/mL) were used to activate WNT, Activin A/TGF, or BMP, respectively. IWR1 (2.5 μM), A-83-01 (5 μM), or LDN193189 (0.4 μM) were used to inhibit WNT, Activin A/TGF, or BMP respectively. A full factorial design was used to generate 27 combinations of media, including a control comprising no small molecules added to the base media (about 20% FBS, FGF2, 2% chicken serum, DMEM/F12).

An immortalized myogenic-origin clone (estimated population doubling level over 100) that had almost no myogenic potential (low percentage of Pax7-positive cells and low level of Pax7 within Pax7 positive cells, and low percentage of myosin heavy chain-positive cells) were plated in a 96 well plate at low density (e.g. 5000 cells/cm2) as triplicates and were cultured in culture media comprising the components as described in FIG. 2 for 2-3 days. Once the cells reached confluency one well was fixed with PFA for staining, and a second well was passaged into a fresh plate with fresh culture media comprising the same components. The third well of the triplicate was plated onto a new plate and was switched to differentiation medium (e.g., culture media comprising 2% horse serum) when cells reached about 80% confluence in their wells. Cells in the were passaged up to 3 passages (i.e., 3 generation) to observe the short term, chronic effect of small molecules.

### 7.2. Example 1: TERT-Immortalized Myoblasts Lose their Myogenic Differentiation Capacity

This experiment was designed to test the hypothesis that TERT-immortalized myoblasts experience reduced differentiation capacity over time in culture. Pax7 expression was used as a marker for myogenic differentiation capacity (i.e., higher Pax7 expression correlates with improved/increased myogenic differentiation capacity).

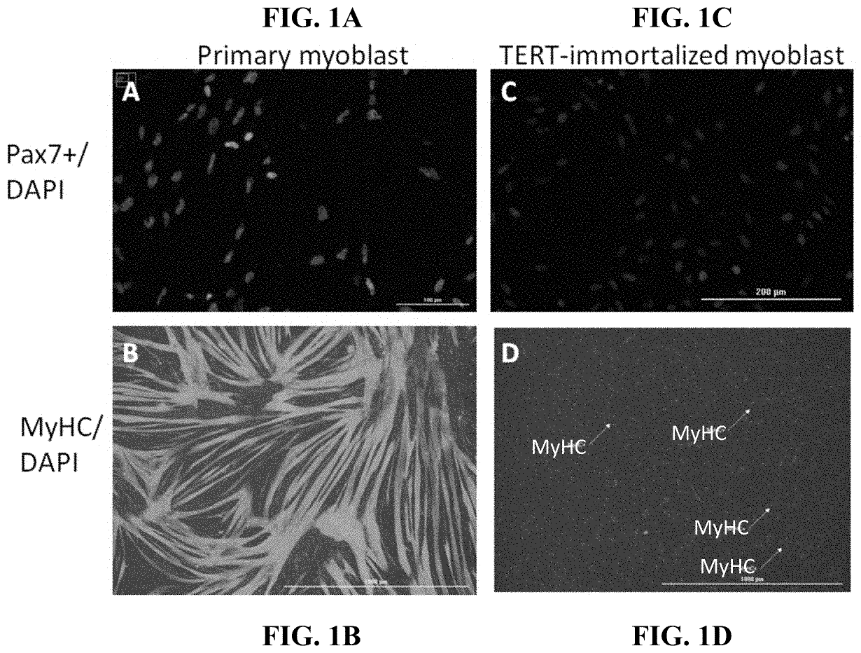

FIGS. 1A-D shows immunofluorescence images comparing Pax 7 expression in primary myoblasts (FIG. 1A) with Pax7 expression in TERT-immortalized myoblasts (FIG. 1C) and MyHC expression in primary myoblasts (FIG. 1B) with MyHC expression in TERT-immortalized myoblasts (FIG. 1D). TERT-immortalized myoblasts were generated by transforming myoblasts with a nucleic acid encoding a telomerase reverse transcriptase (TERT) polypeptide, the catalytic subunit of the telomerase. A monoclonal population of TERT-immortalized myoblasts was used in these experiments. FIG. 1A shows elevated levels of Pax7 expression in primary chicken myoblasts with more than 99% of cells are expressing Pax7. FIG. 1B shows primary chicken myoblasts form robust myotubes as indicated by expression of tubes staining positive for myosin heavy chain (MyHC) (elongated cells). In contrast, TERT-immortalized myoblasts cultured for an estimated PDL greater than 100 had no Pax7 expression (FIG. 1C) and no myotube formation (FIG. 1D; only small faint green signal).

This data suggested that TERT-immortalized myoblasts experience reduced (decreased) myogenic differentiation capacity, in particular following extended culture periods.

### 7.3. Example 2: Full Factorial Media Panel Screen Identifies Components that Enhance Expression of Myogenic Progenitor Markers

This experiment was designed to screen for culture media that enhances expression of myogenic progenitor marker Pax7. Here, Pax7 is used a proxy for improved myogenic differentiation capacity. The immortalized cell line used in this experiment contains few, if any Pax7-positive cells. Therefore, an increase in Pax7-positive cells following contacting with a particular culture media indicates that media is capable of improving myogenic differentiation capacity of an immortalized cell line.

In particular, this full factorial experiment was designed to consist of three factors, each comprising discrete possible values and whose experimental unites take on all possible combinations of these values across all such factors. The study monitored the effect of each factor on the response variable (i.e., Pax7 expression) as well as noting the effects of interactions between factors on the response variable (i.e., Pax7 expression). Without wishing to be bound by theory, the three factors studied represented three pathways crucial in stem cell biology. WNT signaling has been implicated in the control over various types of stem cells. WNT proteins act to maintain the undifferentiated state of stem cells, while other growth factors such as FGF (fibroblast growth factor) and EGF instruct the cells to proliferate. Activin A is a member of the transforming growth factor-β (TGF-β) superfamily, which participates in regulation of several biological processes, including cell differentiation and cell proliferation. Activin A is known to participate in regulation of stem cell maintenance via SMAD-dependent activation transcription markers. Activin A inhibits cell growth and proliferation and activates cell differentiation. BMPs (bone morphogenic proteins) like Activin A, are also members of the TGF-β family. To maintain homeostasis in adults, the BMP signal participates in tissue remodeling and regeneration.

As shown in FIG. 2, 27 media combinations of these three factors were added to base media and contacted with the immortalized cell line. The variables for the factors are presented by (“+”=activation or “−”=inhibition). That is, indicating either activation or inhibition of the WNT, Activin A, and/or BMP signaling pathways by use of agonists and antagonists respectively.

In these experiments, an immortalized myogenic-origin clone having an estimated population doubling level (PDL) over 100 that had a baseline of 0.42% percent of Pax7 positive cells (i.e., % of Pax7-positive cells prior to being contacted with any of the medias in FIG. 2) was then subjected to the 27 different media combinations and cultured for 3 consecutive generations. In each condition, the percentage of Pax7 positive cells was noted at the first generation (passage 1, P1) and third generation (passage 3, P3).

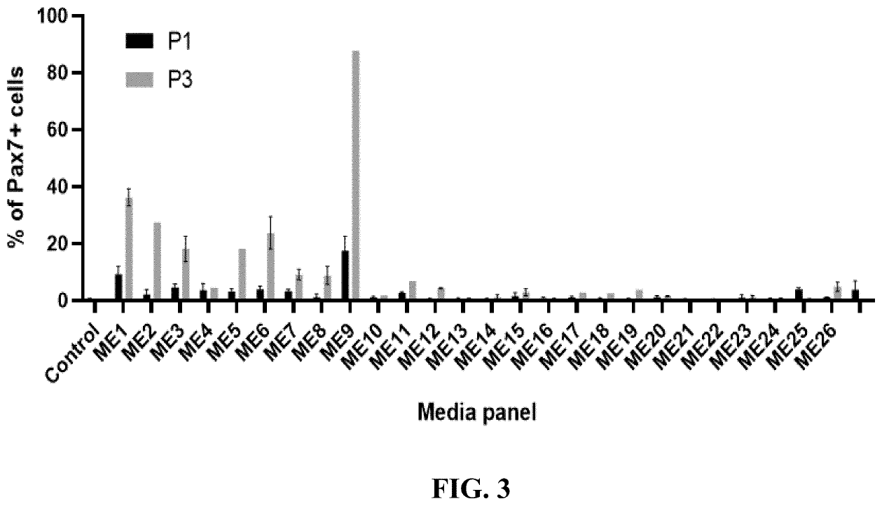

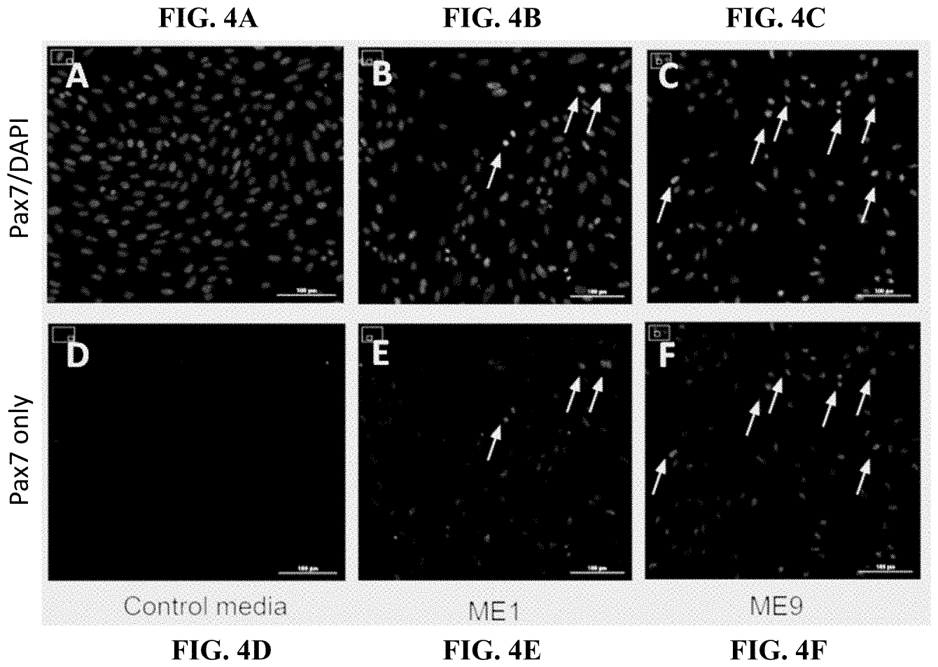

The results in FIG. 3 and FIG. 4A-F show that several culture medias were able to increase myogenic potential as indicated by the enhanced generation of Pax7 positive cells in the population.

ME9 was identified as the most potent inducer of Pax7 expression. ME9 included a WNT activator, a Activin A inhibitor, and a BMP inhibitor. Additional combinations (MEL 3, 6, 7, 11, 24) also induced Pax7 expression. See FIG. 3 and FIGS. 4A-F.

FIG. 4A-F shows representative images of MyHC staining of cell populations with increased numbers of cells expressing myogenic progenitor marker Pax 7 after ME1 and ME9 treatments. All images were taken with a fluorescence microscope at 10× magnification power.

This data showed that ME-9 (WNT activation, Activin A inhibition, and BMP inhibition) has the greatest potential (among the media combinations tested) to improve myogenic potential (i.e., as indicated by the enhanced generation of progenitor marker Pax7 positive cells) and to increase differentiation capacity of late-passage immortalized cells.

### 7.4. Example 3: Full Factorial Media Panel Screen to Identify Factors that Enhance Percent of Cell Area that Express Myogenic Differentiation Marker, Myosin Heavy Chain

The factorial medial panel illustrated in Example 2 was used to determine what combination of available factors enhanced the expression of the myogenic differentiation marker, myosin heavy chain (MyHC).

The same immortalized myogenic-origin clone as used in the Example 2 was also used here. The immortalized myogenic-origin clone had less than 0.1% of MyHC positive cell area expression prior to being subjected to the 27 different media combinations described in FIG. 2. The exposure to the culture media occurred as the cells underwent proliferation. Upon the cells reaching confluence, the media was changed to 2% horse serum, differentiation media for at least 72 hours. The cells were then fixed and stained with MyHC antibodies and examined by fluorescence microscopy.

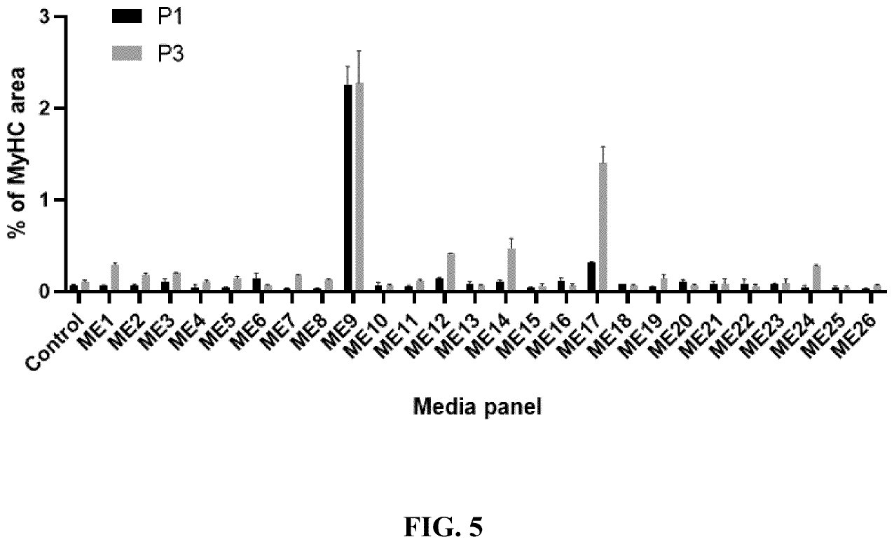

FIG. 5 shows the results of the full factorial media screen identifying components that increase percent of cell area that express myogenic differentiation marker, myosin heavy chain. Cells contacted with culture media comprising ME9 or ME17 during proliferation showed increased differentiation capacity (i.e., increases in cell areas that are MyHC positive) at both P1 and P3. Cells exposed to ME17 experienced increased differentiation capacity over time with greater MyHC positive cell area at P3 compared to P1.

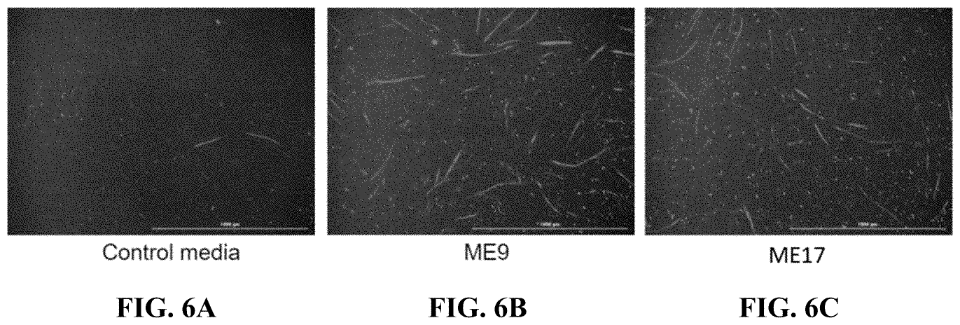

FIG. 6A-C shows representative images of myotube formation in cells exposed to ME9 and ME17. Myotubes are indicated as elongated tendrils staining positive for MyHC. Cells were imaged via florescence microscope at 10× magnification power. Cells were counterstained with DAPI for nuclei and with myosin heavy chain antibody conjugated with Alexa488.

This data further showed that ME-9 (WNT activation, Activin A inhibition, and BMP inhibition) has the greatest potential (among the media combinations tested) to improve differentiation capacity and/or reverse loss of myogenic differentiation potential of late-passage immortalized cells.

### 7.5. Example 4: 7A Primary Cells Transfected with ggMyoD can Form Myotubes and Induce Expression of Downstream Myogenic Factors

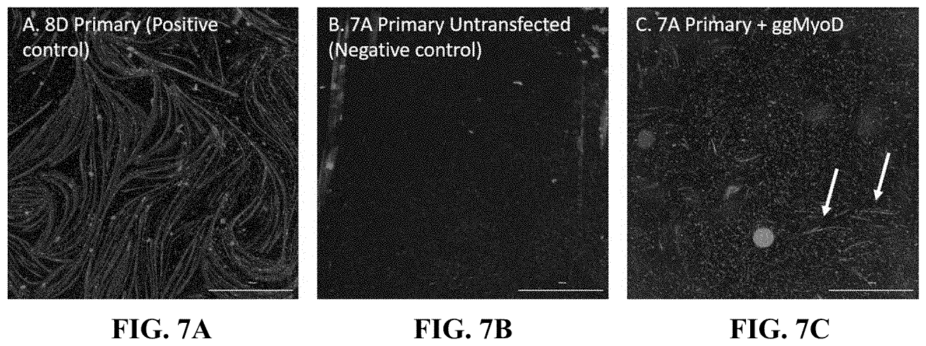

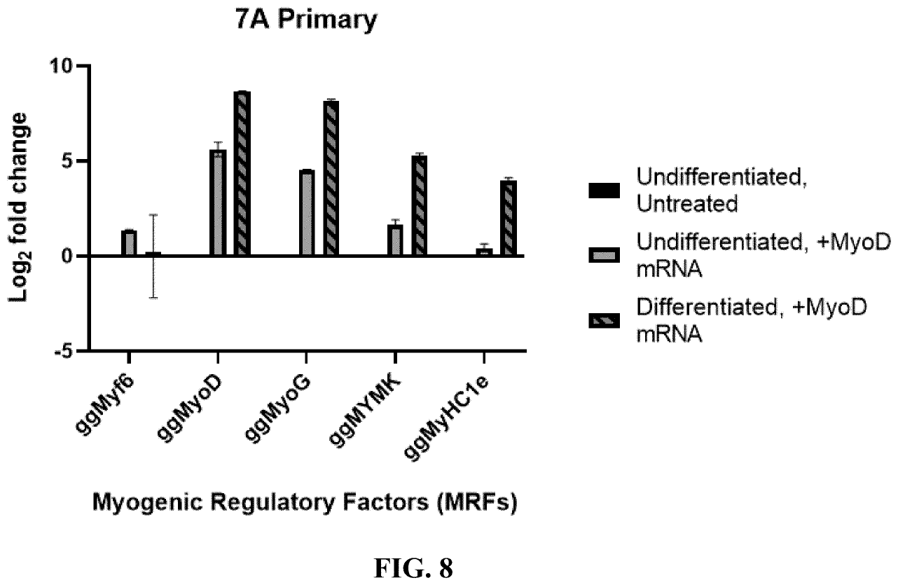

This experiment was designed to test whether primary fibroblast cell transduced with ggMyoD had increased myotube formation and increased expression of downstream myogenic factors as compared to controls. For these experiments, a PhiC31 vector containing a PGK promoter driving expression of a nucleic acid sequence encoding MYOD (from Gallus gallus; ggMYOD) was transfected into 7A primary chicken fibroblasts (see FIG. 7A-7C). These experiments also used direct transfection of mRNA encoding ggMyoD into 7A primary chicken fibroblasts using Lipofectamine RNAiMax (see FIG. 8). In each case, following transduction, cells were differentiated (e.g., by switching to differentiation media (i.e., DMEM/F-12, 2% horse serum)) prior to being assessed for myotube formation and myogenic marker expression.

FIG. 7A is a positive control showing that 8D primary myoblasts form multinucleated myotubes. Myotubes were stained with an APC-conjugated myosin heavy chain antibody. FIG. 7B is a negative control showing that un-transfected 7A primary chicken fibroblasts do not form myotubes. FIG. 7C shows that 7A primary chicken fibroblasts engineered to express ggMYOD start to form myotubes 7 days post-differentiation. Arrows indicate myotubes.

FIG. 8 shows that expression of ggMyoD (transfection of a mRNA encoding ggMyoD) in 7A primary fibroblasts induced expression of endogenous myogenic factors including, ggMyoD, ggMyoG and ggMYMK in both undifferentiated and differentiated cells as compared to untreated control cells. Cells cultured in differentiation media showed a further increase in expression of endogenous myogenic factors and as well as terminal differentiation marker ggMyHC1e. This data showed that transducing primary chicken fibroblasts with mRNA encoding ggMyoD is sufficient to induce expression of endogenous myogenic marks (see FIG. 8).

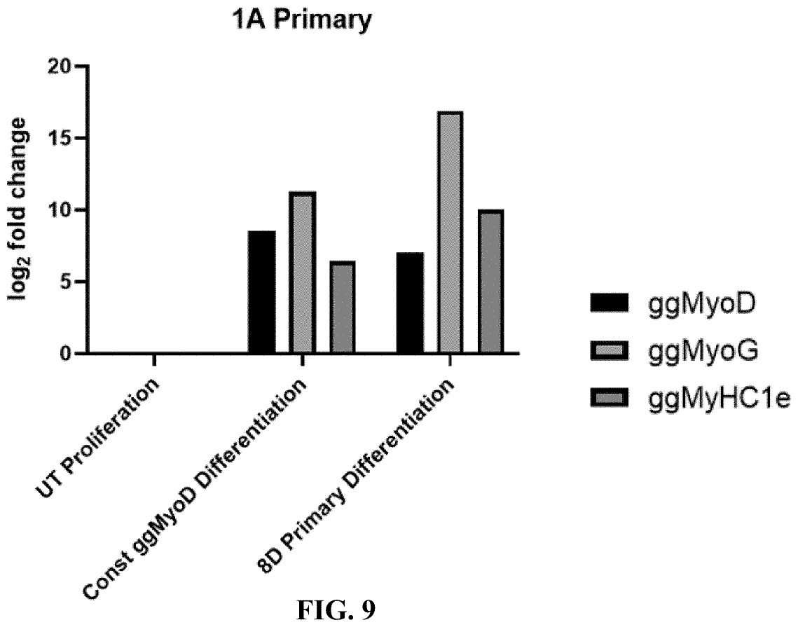

In a similar experiment, a vector containing a PGK promoter driving expression of a nucleic acid encoding ggMyoD, including a Puromycin selection cassette, was transfected into 1A primary chicken fibroblasts using Lipofectamine 3000 (see FIG. 9). Following transfection, cells were selected in 0.5 ug/ml puromycin for 4 days. FIG. 9 shows that incorporating a polynucleotide encoding ggMyoD into the genome of a primary chicken fibroblast induces expression of endogenous myogenic factors (MyoG and MyHC1). Following selection, the cells were differentiated according to the methods described herein. Control 8D primary myoblasts differentiated and expressed high levels of MyoD, MyoG and MYHC1e. Similarly, ggMyoD overexpression in primary chicken fibroblasts induced high levels of ggMyoG and ggMyHC1e in the differentiated cells.

Overall, this data showed that engineering chicken primary fibroblast cells to express ggMYOD can be used as a method to improve myotube formation and increase expression of downstream myogenic factors, thereby improving myogenic differentiation capacity.

### 7.6. Example 5: Overexpression of Myogenic Factors PAX7, MYOD, and MEF2B Enhanced Myogenicity in Immortal Myoblasts

This experiment was designed to assess myogenicity of immortalized fibroblasts following transduction with a vector including a nucleic acid sequence encoding ggMYOD or nucleic acid sequences encoding PAX7, MYOD, and MEF2B (“7MM”). In each instance, the immortalized fibroblasts, including controls, were cultured in ME9 media containing (an Activin A inhibitor, a BMP inhibitor, and a WNT activator). Each vector included a neomycin selection cassette to enable selection. For this experiment, 8D myoblasts previously immortalized using TERT (“8D TERT”) were transduced and then selected.

Parental 8D TERT myoblasts (control), 8D TERT myoblasts transduced with ggMyoD, and 8D TERT myoblasts transduced with 7MM were each seeded in a 96-well plate in ME9. Once the cells reached confluence, media was replaced with differentiation media (2% horse serum) and after three days the presence of myotubes was assessed by myosin heavy chain staining.

FIG. 10A-C shows that parental 8D TERT myoblasts (control) had poor myogenicity even in the presence of ME9. In contrast, overexpression of ggMyoD (FIG. 10A) or PAX7/MEF2B/MYOD (FIG. 10B) increased myogenic differentiation capacity (as indicated by myotube formation) when combined with ME9. In FIGS. 10A-C MyHC staining was used to aid identification of myotubes.

This data showed that culturing cell lines transformed with a polynucleotide encoding at least a first myogenic regulatory factor polypeptide (MYOD) or a combination of myogenic regulatory factor polypeptides (PAX7, MYOD, and MEF2B (“7MM”) in culture medium comprising an Activin A inhibitor, a BMP inhibitor, and a WNT activator increases myogenic differentiation and/or reverses the loss of myogenicity in immortalized, aged 8D myoblasts.

### 7.7. Example 6: ggMyoD mRNA Induced Expression of Downstream Myogenic Factors in an Earlier Passage TERT-Immortalized 7A Chicken Fibroblasts

This experiment was designed to assess expression of endogenous downstream myogenic factors (ggMyoG, ggMYMK, and ggMyHC1e) following transfection with mRNA encoding ggMYOD in early passage (PDL ˜40) immortalized cells. For this experiment, a mRNA encoding ggMyoD was transfected into TERT-immortalized 7A chicken fibroblasts (“7A TERT”) using Lipofectamine RNAiMax. TERT-immortalized 7A fibroblasts were cultured for PDL ˜40 prior to transfection. Following transfection, fibroblasts were exposed either to differentiation media (i.e., DMEM/F-12, 2% horse serum) or proliferation media (i.e., any of the proliferation medias described herein; referred to as “undifferentiated” sample (e.g., see culture medias described in FIG. 2)) prior to being assessed for myogenic marker expression using qPCR.

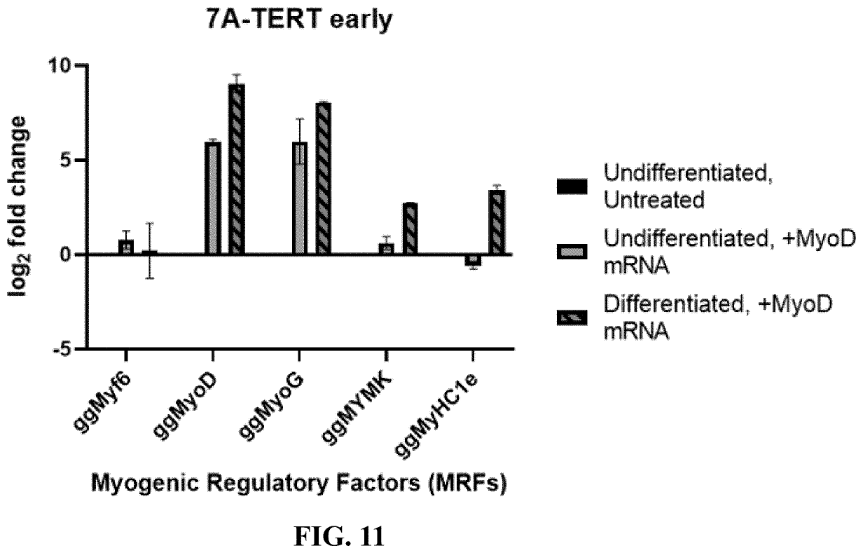

FIG. 11 shows that expression of ggMyoD in 7A TERT fibroblasts (PDL˜40) induced expression of endogenous myogenic factors, ggMyoD and ggMyoG, in both differentiated and undifferentiated cells compared to untreated cells. Cells cultured in differentiation media showed a further increase in ggMyoD and ggMyoG expression as well as an increase in terminal differentiation markers ggMYMK and ggMyHC1e, suggesting that these cells can fully differentiate into myotubes.

### 7.8. Example 7: TERT-Immortalized Late Passage 7A Chicken Fibroblasts Stably Transfected with ggMYOD do not Express Downstream Myogenic Factors

This experiment was designed to assess expression of endogenous downstream myogenic factors (ggMyoG, ggMYMK, and ggMyHC1e) following transfection with a vector encoding ggMYOD in late passage (PDL >100) immortalized fibroblast cells compared to primary fibroblasts. TERT-immortalized 7A fibroblasts were cultured for PDL >100 prior to transfection. For these experiments, a vector including a PGK promoter driving expression of a nucleic acid sequence encoding ggMYOD was transfected into TERT-immortalized 7A chicken fibroblasts (“7A TERT”) or primary fibroblasts using Lipofectamine 3000. Each vector included a puromycin selection cassette to enable selection. Following transfection and selection (0.5 mg/mL puromycin for 4 days), fibroblasts were exposed either to differentiation media (i.e., DMEM/F-12, 2% horse serum) or proliferation media (i.e., ME9; referred to as “undifferentiated” sample (see also culture medias described in FIG. 2)) prior to being assessed for myogenic marker expression using qPCR.

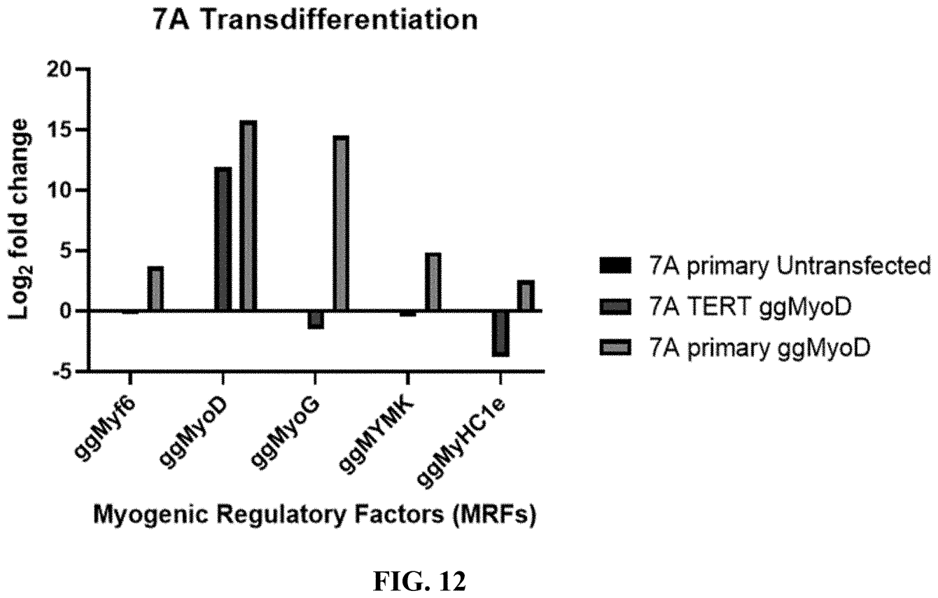

FIG. 12 shows that late passage 7A chicken fibroblasts immortalized by overexpressing ggTERT (PDL >100) and stably transfected with ggMYOD did not express endogenous downstream myogenic factors. In contrast, 7A chicken primary cells stably transfected with ggMYOD did express endogenous downstream myogenic factors.

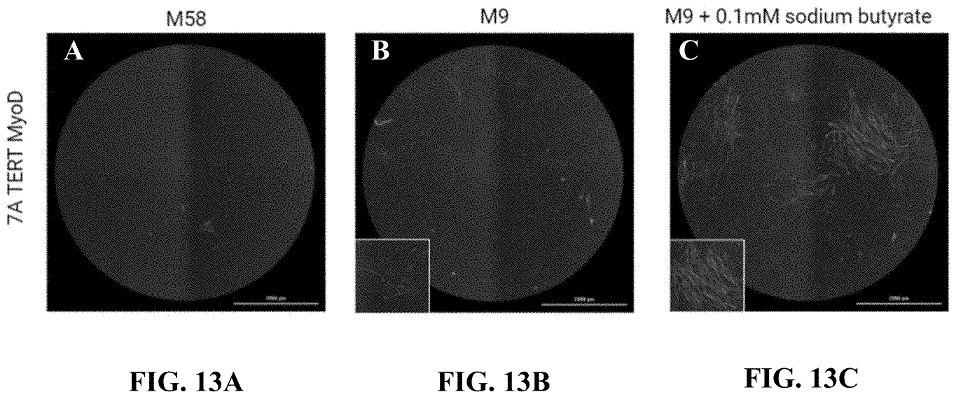

To assess whether the late passage immortalized 7A chicken fibroblasts could be induced to form myotubes, the late passage immortalized 7A chicken fibroblasts stably transfected with ggMYOD (as described above; referred to as 7A TERT ggMYOD) were cultured in media: ME58, ME9, and ME9+0.1 mM sodium butyrate. The results are shown in FIG. 13A-C. In FIGS. 13A-C MyHC staining was used to aid identification of myotubes.

FIG. 13A shows that 7A TERT ggMyoD cells cultured in media 58 (ME58; ME58 is DMEM/F12, about 20% FBS, and about 5% chicken serum) did not improve the differentiation capacity. FIG. 13B shows an improvement in differentiation capacity for the 7A TERT ggMyoD cells when cultured in ME9 as compared to ME58 (see, FIG. 2). Surprisingly, as shown in FIG. 13C, 7A TERT ggMyoD cells cultured in ME9 in the presence of sodium butyrate showed dramatic improvement in differentiation capacity.

Similar to primary fibroblasts, an early generation of a TERT-immortalized chicken fibroblast line improved myogenic differentiation capacity using the methods described herein (FIG. 11). However, unexpectedly, MyoD-overexpression in an old TERT-immortalized chicken fibroblast line was not sufficient to improve myogenic differentiation capacity (FIG. 12). When these TERT-immortalized MyoD expressing fibroblasts were exposed to ME9 there was a small increase in myogenic differentiation capacity (FIG. 13). When exposed to sodium butyrate the TERT-immortalized MyoD expressing fibroblasts experienced a significant increase in myogenic differentiation capacity (as measured by myotube formation) as compared to cells not exposed to sodium butyrate (FIG. 13).

This data showed that ME9+sodium butyrate can be used to further improve differentiation capacity of an immortalized cell line.

### 7.9. Example 8: Transduction of TERT-Immortalized 7A Fibroblasts with PAX7/MEF2B/MYOD (7MM) Enhances Transdifferentiation into Myoblasts

This experiment was designed to assess differentiation capacity in TERT-immortalized 7A chicken fibroblasts following transduction with ggMYOD or PAX7/MEF2B/MYOD (“7MM”). Differentiation capacity was assessed using myotube formation.

For this experiment, the TERT-immortalized 7A chicken fibroblasts (“7A TERT” fibroblasts) were transfected with a vector including a nucleic acid sequence encoding ggMYOD or 7MM and a neomycin selection cassette. Following transfection and selection (using 0.8 mg/mL neomycin), fibroblasts were exposed either to ME58 or M9. Once the fibroblasts reached confluence, media was replaced with differentiation medium (2% horse serum). After three days in differentiation media, myotube formation was assessed using myosin heavy chain staining (green) to stain the myotubes. Once the cells reached confluence, fibroblasts were cultured in differentiation medium (i.e., DMEM/F-12 containing 2% horse serum). Non-transfected primary myoblasts (“8D primary”) were used as a positive control.

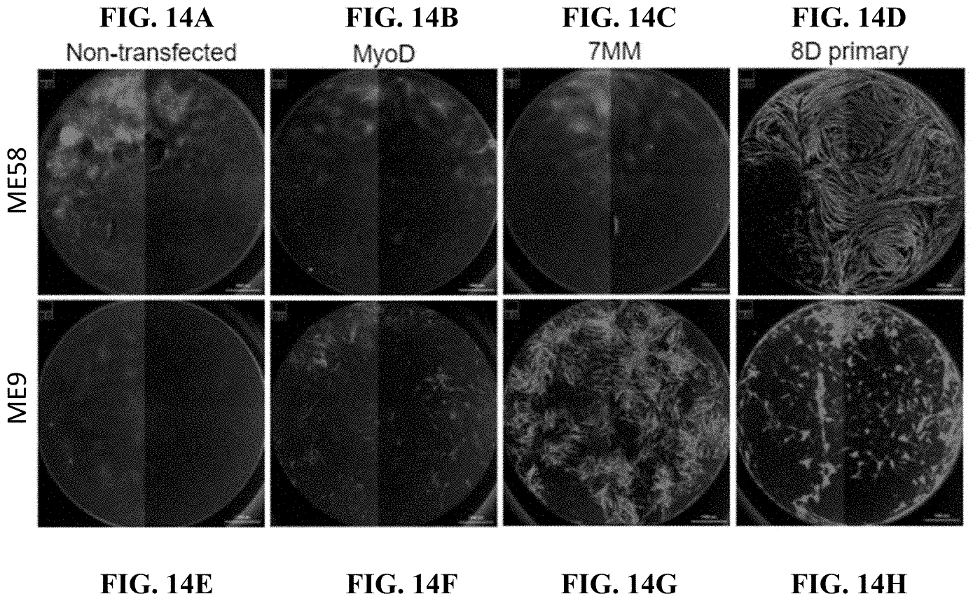

FIGS. 14A-H shows 7A TERT fibroblasts non-transfected (“non-transfected”) control did not form myotubes in either ME58 (FIG. 14A) or ME9 media (FIG. 14E). In FIGS. 14A-H MyHC staining was used to aid identification of myotubes. As a positive control, 8D primary myoblasts formed myotubes in both ME58 (FIG. 14D) and ME9 (FIG. 14H), as expected. 7A TERT fibroblasts stably transfected with ggMYOD showed some myotube formation in ME9 (FIG. 14F) but not in M58 (FIG. 14B). 7A TERT fibroblasts stably transfected with 7MM showed robust myotube formation in ME9 (FIG. 14G) but no myotube formation in ME58 (FIG. 14C).

This data suggests that transduction with MyoD alone was insufficient to induce robust myotube formation in a late passage, TERT-immortalized fibroblast cell line. In contrast, transduction with 7MM in combination with ME9 was extremely effective in inducing myotube formation in the late-passage, TERT-immortalized fibroblast cell line, and therefore, can be used to improve differentiation capacity of an immortalized cell line.

### 7.10. Example 9: Transduction of TERT-Immortalized 8G Bovine Fibroblasts with MYOD Enhances Transdifferentiation into Myoblasts

This experiment was designed to assess differentiation capacity in TERT-immortalized 8G bovine fibroblasts following transduction with btMYOD. Differentiation capacity was assessed using myotube formation.

For this experiment, the TERT-immortalized 8G bovine fibroblasts (“8G TERT” fibroblasts) were transfected with a vector including a nucleic acid sequence encoding btMYOD and a neomycin selection cassette. Following transfection and selection (using 0.8 mg/mL neomycin), fibroblasts were exposed to ME9. Once the fibroblasts reached confluence, media was replaced with differentiation medium (2% horse serum). After three days in differentiation media, myotube formation was assessed using myosin heavy chain staining (green) to stain the myotubes. Non-transfected 8G TERT fibroblasts were used as a control.

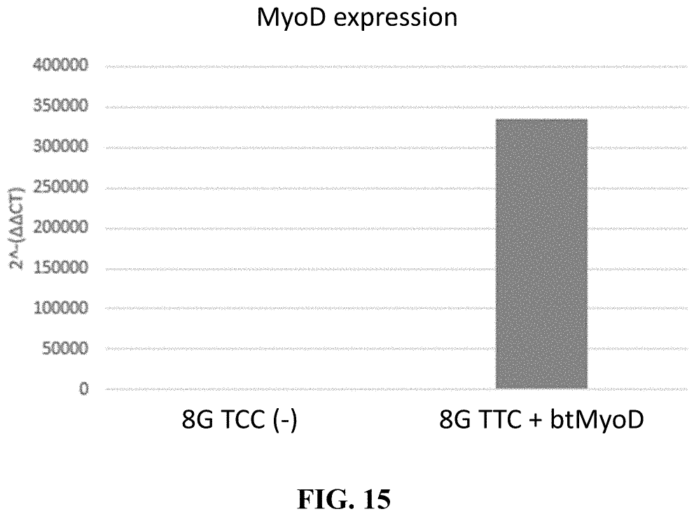

FIG. 15 shows RNA expression of MyoD in 8G TERT fibroblasts non-transfected (“8G TCC”) controls and 8G TERT fibroblasts transfected with btMYOD (“8G TCC+MyoD”). RNA was extracted according to the methods described herein and qRT-PCR was used to assess expression levels. FIG. 15 shows that only 8G TERT fibroblasts transfected with btMYOD showed expression of btMYOD RNA.

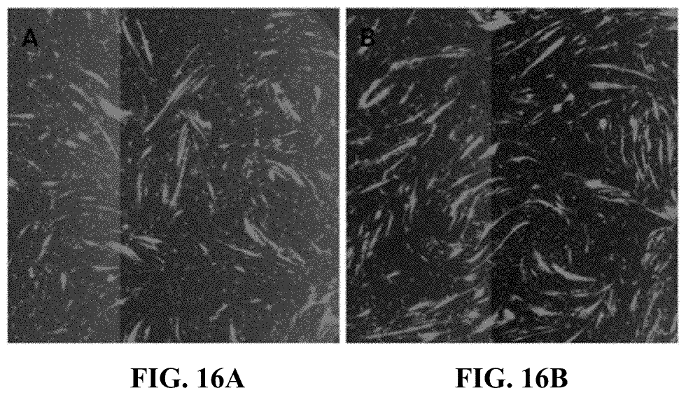

FIGS. 16A-16B shows 8G TERT fibroblasts transfect with btMYOD formed myotubes (as indicated using myosin heavy chain) in ME9 media. In FIGS. 15A-B MyHC staining was used to aid identification of myotubes. FIG. 16A shows transfected 8G TERT fibroblasts grown in differentiation media. FIG. 16B shows transfected 8G TERT fibroblasts grown in proliferation media.

This data suggests that transduction of MyoD induced robust myotube formation in a late passage, TERT-immortalized bovine fibroblast cell line, and therefore, can be used to improve myogenic differentiation capacity of an immortalized cell line.

### 8. ADDITIONAL EMBODIMENTS

Embodiment 1. A method for improving differentiation capacity of a cell line, comprising: (a) isolating a monoculture of cells from skin or muscle to form a cell line; (b) immortalizing the cell line; (c) exposing the immortalized cell line to at least one Activin A inhibitor and at least one BMP inhibitor, one or more myogenic regulatory factors, or any combination thereof.

Embodiment 2. The method of embodiment 1, wherein the immortalizing step comprises transforming the cell line with telomerase reverse transcriptase (TERT).

Embodiment 3. A method for improving differentiation capacity of an immortalized cell line comprising: exposing the cell line to at least one Activin A inhibitor and at least one BMP inhibitor.

Embodiment 4. The method of embodiment 1, further comprising transforming the immortalized cell line with one or more myogenic regulatory factors producing a recombinant cell line expressing the one or more myogenic regulatory factors.

Embodiment 5. A method for improving differentiation capacity of an immortalized cell line comprising: transforming the fibroblast cell line with one or more myogenic regulatory factors producing a recombinant cell line expressing the one or more myogenic regulatory factors.

Embodiment 6. The method of embodiment 5, further comprising exposing the immortalized cell line to at least one Activin A inhibitor and at least one BMP inhibitor.

Embodiment 7. The method of any one of embodiments 1-6, wherein the one or more myogenic regulatory factors are selected from: MYOD, MYOG, MEF2B, PAX7, PAX3, and PITX1, or any combination thereof.

Embodiment 8. The method of embodiment 7, wherein the one or more myogenic regulatory factor comprises MYOD or a fragment thereof.

Embodiment 9. The method of embodiment 7, wherein the one or more myogenic regulatory factor comprises PAX7 or a fragment thereof.

Embodiment 10. The method of embodiment 7, wherein the one or more myogenic regulatory factors comprise: PAX7 (or a fragment thereof), MEF2B (or a fragment thereof), and MYOD (or a fragment thereof), or any combination thereof.

Embodiment 11. The method of any one of embodiments 2-10, wherein the one or more myogenic regulatory factors are constitutively expressed in the immortalized cell line.

Embodiment 12. The method of any one of embodiments 2-11, wherein exposing the immortalized cell line to the one or more myogenic regulatory factors comprises introducing a nucleic acid construct into the immortalized cell, wherein the nucleic acid construct comprises one or more nucleic acid sequences encoding the one or more myogenic regulatory factors.

Embodiment 13. The method of embodiment 12, wherein introducing the nucleic acid sequences encoding the one or more myogenic regulatory factors into the immortalized cell line comprises establishing an immortalized cell line that stably expresses the one or more myogenic regulatory factors.

Embodiment 14. The method of embodiment 12, wherein introducing the nucleic acid sequence encoding the one or more myogenic regulatory factors into the immortalized cell line comprises incorporating the nucleic acid sequence into the genome of the immortalized cell line.

Embodiment 15. The method of any one of embodiments 1-11, wherein the one or more myogenic regulatory factors are introduced into the immortalized cell using mRNA encoding the one or more myogenic regulatory factors.

Embodiment 16. The method of any of embodiments 1-15, further comprising exposing the cell line or immortalized cell line to at least one WNT activator.

Embodiment 17. The method of embodiment 16, wherein the WNT activator is CHIR99021 or WNT1a.

Embodiment 18. The method of any of embodiments 1-17, wherein the Activin A inhibitor is A-83-01 or Follistatin, and the BMP inhibitor is LDN193189 or Noggin.

Embodiment 19. The method of embodiment 18, wherein the WNT activator is WNT1a, the Activin A inhibitor is Follistatin, and the BMP inhibitor is Noggin.

Embodiment 20. The method of any one of embodiments 1-19, further comprising exposing the cell line or immortalized cell line to a reagent for epigenetic modulation.

Embodiment 21. The method of embodiment 20, wherein the epigenetic modulator is a histone deacetylase inhibitor.

Embodiment 22. The method of embodiment 21, wherein the histone deacetylase inhibitor is sodium butyrate.

Embodiment 23. The method of any one of embodiments 1-22, wherein the cell line or immortalized cell line are from a livestock, poultry, game, or aquatic animal species.

Embodiment 24. The method of any one of embodiments 1-22, wherein the cell line or immortalized cell line are from a chicken, duck, or turkey.

Embodiment 25. The method of any one of embodiments 1-22, wherein the cell line or immortalized cell line are from a fish.

Embodiment 26. The method of any one of embodiments 1-22, wherein the cell line or immortalized cell line are from a livestock species.

Embodiment 27. The method of embodiment 26, wherein the livestock species is porcine or bovine.

Embodiment 28. The method of any one of embodiments 1-27, wherein the cell line or immortalized cell line is a fibroblast cell line.

Embodiment 29. The method of any one of embodiments 1-28, wherein the cell line or immortalized cells are not stem cells.

Embodiment 30. The method of any one of embodiments 1-29, wherein prior to exposing the cell line or immortalized cell line to the methods of any one of embodiments 1-29, the cell line or immortalized cell line comprises a population doubling level (PDL) of at least 60.

Embodiment 31. The method of embodiment 30, wherein prior to exposing the cell line or immortalized cell line to the methods of any one of embodiments 1-29, the cell line or immortalized cell line comprises less than 5% Pax7+ cells and/or less than 0.2% MyHC1+ cells.

Embodiment 32. The method of any one of embodiments 1-31, wherein increased differentiation capacity comprises increased Pax7 expression, increased MyHC1 expression, and/or increased myotube formation.

Embodiment 33. The method of embodiment 32, wherein the cell line or immortalized cell line exhibits increased Pax7 expression, increased MyHC1 expression, and/or increased myotube formation, as compared to a cell line or immortalized cell line that are not exposed to the at least one Activin A inhibitor, at least one BMP inhibitor, at least one WNT activator, one or more myogenic regulatory factors, epigenetic modulator, or any combination thereof.

Embodiment 34. The method of any one of embodiments 2-33, wherein the immortalized cell line exhibits an increased differentiation capacity after at least 60 passages compared with an immortalized cell line not exposed to the at least one Activin A inhibitor, at least one BMP inhibitor, at least one WNT activator, one or more myogenic regulatory factors, epigenetic modulator, or any combination thereof.

Embodiment 35. The method of any one of embodiments 1-34, further comprising the step of adapting the cell line for suspension culture.

Embodiment 36. The method of embodiment 35, wherein adapting the cell line for suspension culture comprises a period of time of about 30 days or about 10 or more passages.

Embodiment 37. The method of any one of embodiments 2-36, further comprising inducing myogenic-specific differentiation.

Embodiment 38. The method of embodiment 34, wherein inducing myogenic-specific differentiation comprises generating myocytes, myoblasts, myotubes, multinucleated myotubes, satellite cells, skeletal muscle fibers, or any combination thereof.

Embodiment 39. An immortalized fibroblast cell line produced by any of the methods of embodiments 1-36.

Embodiment 40. A population of immortalized cells produced by any of the methods of embodiments 1-36.

Embodiment 41. The population of immortalized cells of embodiment 40, wherein the population of immortalized cells exhibits increased Pax7 expression, increased MyHC1 expression, and/or increased myotube formation, as compared to a population of immortalized cells that are not exposed to the at least one Activin A inhibitor, at least one BMP inhibitor, at least one WNT activator, one or more myogenic regulatory factors, epigenetic modulator, or any combination thereof.

Embodiment 42. A population of myocytes, myoblasts, myotubes, multinucleated myotubes, satellite cells, skeletal muscle fibers, or any combination thereof produced by any of the methods of embodiments 1-38.

Embodiment 43. An in vitro method for producing cultured muscle tissue (cell based meat product), comprising: exposing the immortalized cell line to at least one Activin A inhibitor and at least one BMP inhibitor; transforming the immortalized cell line with one or more myogenic regulatory factors producing a recombinant cell line expressing the one or more myogenic regulatory factors; and inducing myogenic specific differentiation.

Embodiment 44. The method of embodiment 43, wherein the one or more myogenic regulatory factors are selected from: MYOD, MYOG, MEF2B, PAX7, PAX3, and PITX1.

Embodiment 45. The method of embodiment 44, wherein the one or more myogenic regulatory factor comprises MYOD or a fragment thereof.

Embodiment 46. The method of embodiment 44, wherein the one or more myogenic regulatory factors comprise: PAX7 (or a fragment thereof), MEF2B (or a fragment thereof), and MYOD (or a fragment thereof).

Embodiment 47. The method of any one of embodiments 44-46, further comprising exposing the cell line or immortalized cell line to at least one WNT activator.

Embodiment 48. The method of embodiment 47, wherein the WNT activator is CHIR99021 or WNT1a.

Embodiment 49. The method of any one of embodiments 43-48, wherein the Activin A inhibitor is A-83-01 or Follistatin, and the BMP inhibitor is LDN193189 or Noggin.

Embodiment 50. The method of embodiment 49, wherein the WNT activator is WNT1a, the Activin A inhibitor is Follistatin, and the BMP inhibitor is Noggin.

Embodiment 51. The method of any one of embodiments 43-50, further comprising exposing the cell line or immortalized cell line to a reagent for epigenetic modulation.

Embodiment 52. The method of embodiment 51, wherein the epigenetic modulator is a histone deacetylase inhibitor.

Embodiment 53. The method of embodiment 52, wherein the histone deacetylase inhibitor is sodium butyrate.

Embodiment 54. The method of any one of embodiments 43-53, wherein the cell line or immortalized cell line are from a livestock, poultry, game, or aquatic animal species.

Embodiment 55. The method of any one of embodiments 43-53, wherein the cell line or immortalized cell line are from a chicken, duck, or turkey.

Embodiment 56. The method of any one of embodiments 43-53, wherein the cell line or immortalized cell line are from a fish.

Embodiment 57. The method of any one of embodiments 43-53, wherein the cell line or immortalized cell line are from a livestock species.

Embodiment 58. The method of embodiment 57, wherein the livestock species is porcine or bovine.

Embodiment 59. The method of any one of embodiments 43-58, wherein the cell line or immortalized cell line is a fibroblast cell line.

Embodiment 60. The method of any one of embodiments 43-59, wherein inducing myogenic-specific differentiation comprises generating myocytes, myoblasts, myotubes, multinucleated myotubes, satellite cells, skeletal muscle fibers, or any combination thereof.

Embodiment 61. A population of myocytes, myoblasts, myotubes, multinucleated myotubes, satellite cells, skeletal muscle fibers, or any combination thereof produced by any of the methods of embodiments 43-60.

Embodiment 62. A kit comprising: at least one Activin A inhibitor; at least one BMP inhibitor; and at least one WNT activator.

Embodiment 63. The kit of embodiment 62, further comprising one or more myogenic regulators.

Embodiment 64. The kit of embodiment 62 or 63, further comprising an epigenetic modulator.

Embodiment 65. The kit of any one of embodiments 62-64, further comprising the immortalized fibroblast cell line of embodiment 39 or any of the populations of cells of embodiments 40-42, or embodiment 61.

Embodiment 66. The kit of any one of embodiments 62-65, further comprising instructions for performing any of the methods of embodiments 1-38 or embodiments 43-60.

Embodiment 67. A kit for improving myogenic differentiation capacity of a cell line or an immortalized cell line comprising: at least a first Activin A inhibitor; at least a first BMP inhibitor, at least a first WNT activator, or a combination thereof.

Embodiment 68. The kit of embodiment 67, wherein the kit further comprises a first myogenic regulatory polypeptide, a second myogenic regulatory polypeptide, a third myogenic regulatory polypeptide, or a combination thereof.

Embodiment 69. The kit of embodiment 68, wherein the first myogenic regulatory polypeptide, the second myogenic regulatory polypeptide, and/or the third myogenic regulatory polypeptide is selected from MYOD, MYOG, MEF2B, PAX7, PAX3, and PITX1.

Embodiment 70. The kit of any one of embodiments 67-69, further comprising a histone deacetylase inhibitor.

Embodiment 71. The kit of embodiment 70, wherein the histone deacetylase inhibitor is sodium butyrate.

Embodiment 72. The kit of any one of embodiments 67-71, further comprising any of the immortalized fibroblast cell lines described herein.

Embodiment 73. The kit of any one of embodiments 67-72, further comprising instructions to perform any of the methods described herein.

Embodiment 74. A cell culture media for improving myogenic differentiation capacity of a cell line or an immortalized cell line, the cell culture media comprising: at least a first Activin A inhibitor; at least a first BMP inhibitor, at least a first WNT activator, or a combination thereof.

Embodiment 75. The cell culture media of embodiment 74, further comprising a histone deacetylase inhibitor.

Embodiment 76. The cell culture media of embodiment 75, wherein the histone deacetylase inhibitor is sodium butyrate.

Embodiment 77. A method for improving myogenic differentiation capacity of a late passage cell line or immortalized cell line, the method comprising:
(a) transforming introducing into, or incorporating into the genome of, a cell of the late passage cell line with a polynucleotide encoding at least a first myogenic regulatory factor polypeptide, thereby producing a recombinant immortalized cell line expressing the at least first myogenic regulatory factor polypeptide; and
(b) inducing myogenic specific differentiation comprises inducing formation of myocytes and myotubes,
thereby improving the cell line's myogenic differentiation capacity as compared to a late passage cell line control or an immortalized cell line control.

Embodiment 78. The method of embodiment 77, further comprising contacting the late passage cell line or immortalized cell line with a cell culture media comprising:
(i) at least a first Activin A inhibitor,
(ii) at least a first BMP inhibitor, or
(iii) at least a first WNT activator,
or a combination thereof.

Embodiment 79. The method of embodiment 77 or 78, wherein the late passage cell line has lost myogenic differentiation capacity

Embodiment 80. The method of any one of embodiments 77-79, wherein the at least first myogenic regulatory factor is selected from: MYOD, MYOG, MEF2B, PAX7, PAX3, and PITX1

Embodiment 81. The method of any one of embodiments 77-80, wherein the polynucleotide comprising the first myogenic regulatory factor polypeptide further comprises a nucleic acid sequence encoding a second myogenic regulatory factor polypeptide, a nucleic acid sequence encoding a third myogenic regulatory factor polypeptide, or a combination thereof.

Embodiment 82. The method of embodiment 81, wherein the first myogenic regulatory factor polypeptide is a PAX7 polypeptide or a fragment thereof, the second myogenic regulatory factor polypeptide is a MEF2B polypeptide or a fragment thereof, and the third myogenic regulatory factor polypeptide is a MYOD polypeptide or a fragment thereof).

Embodiment 83. The method of any one of embodiments 78-82, wherein the Activin A inhibitor is selected from: A-83-01, E-616542, SB431542, TGFβRI-IN-3, R-268712, Follistatin, and Follistatin-like-3

Embodiment 84. The method of any one of embodiments 78-83, wherein the BMP inhibitor is selected from: LDN193189, Noggin, Chrodin, and Gremlin.

Embodiment 85. The method of any one of embodiments 78-84, wherein the WNT activator is selected from: CHIR99021, BIO, AZD1080, WNT1a, WNT3a, WNT4, and WNT7.

Embodiment 86. The method of any one of embodiments 78-85, further comprising contacting the cell line with a culture media comprising a histone deacetylase inhibitor.

Embodiment 87. The method of embodiment 86, wherein the histone deacetylase inhibitor is sodium butyrate.

Embodiment 88. The method of any one of embodiments 77-87, wherein the cell line are from a species selected from: poultry, livestock, game, or aquatic animal species

Embodiment 89. The method of any one of embodiments 77-88, wherein the cell line or the late passage cell line is a fibroblast cell line

Embodiment 90. The method of any one of embodiments 77-89, wherein inducing myogenic-specific differentiation comprises contacting the cell line or immortalized cell line with a differentiation medium

Embodiment 91. An in vitro method for producing a cell-based meat product, comprising: forming the myocytes, myoblasts, myotubes, or a combination thereof, from any one of embodiments 77-90.

Embodiment 92. A method for improving myogenic differentiation capacity of a cell line, comprising:
(a) isolating a population of cells from skin or muscle tissue to form a cell line;
(b) immortalizing the cell line;
(c) contacting the cell line with a culture media comprising:
   (i) at least a first Activin A inhibitor,
   (ii) at least a first BMP inhibitor, or
   (iii) at least a first WNT activator, or
   a combination thereof; and
(d) inducing myogenic specific differentiation, comprising inducing formation of myocytes, myoblasts, myotubes, or a combination thereof,
thereby improving the cell line's myogenic differentiation capacity as compared to a control.

Embodiment 93. The method of embodiment 92, wherein the immortalizing step comprises introducing into, or incorporating into the genome of, a cell of the cell line a polynucleotide encoding a telomerase reverse transcriptase (TERT) polypeptide, thereby generating an immortalized cell line.

Embodiment 94. A method for improving myogenic differentiation capacity of a late passage cell line comprising:
(a) contacting the late passage cell line with a culture media comprising:
   (i) at least a first Activin A inhibitor,
   (ii) at least a first BMP inhibitor, or
   (iii) at least a first WNT activator, or a combination thereof,
(b) inducing myogenic specific differentiation, comprising inducing formation of myocytes, myoblasts, myotubes, or a combination thereof,
thereby improving the late passage cell line's myogenic differentiation capacity as compared to a late passage control.

Embodiment 95. A method for restoring myogenic differentiation capacity of a late passage cell line comprising:
(a) contacting the late passage cell line with a culture media comprising:
   (i) at least a first Activin A inhibitor,
   (ii) at least a first BMP inhibitor, or
   (iii) at least a first WNT activator, or a combination thereof; and
(b) inducing myogenic specific differentiation, comprising inducing formation of myocytes, myoblasts, myotubes, or a combination thereof,
wherein restoring the late passage cell line myogenic differentiation capacity as compared to a late passage control.

Embodiment 96. The method of any one of embodiments 92-95, further comprising introducing into, or incorporating into the genome of, a cell of the cell line, a cell of the immortalized cell line, or a cell of the late passage cell line a polynucleotide encoding at least a first myogenic regulatory factor polypeptide, thereby producing a recombinant cell line expressing the at least first myogenic regulatory factor polypeptide.

Embodiment 97. The method of embodiment 96, wherein the at least first myogenic regulatory factor is selected from: MYOD, MYOG, MEF2B, PAX7, PAX3, and PITX1.

Embodiment 98. The method of embodiment 96 or 97, wherein the polynucleotide comprising the first myogenic regulatory factor polypeptide further comprises a nucleic acid sequence encoding a second myogenic regulatory factor polypeptide, a nucleic acid sequence encoding a third myogenic regulatory factor polypeptide, or a combination thereof, wherein the first, the second, and the third myogenic regulatory factor polypeptides are selected from MYOD, MYOG, MEF2B, PAX7, PAX3, and PITX1.

Embodiment 99. The method of embodiment 98, wherein the first myogenic regulatory factor polypeptide is a PAX7 polypeptide or a fragment thereof, the second myogenic regulatory factor polypeptide is a MEF2B polypeptide or a fragment thereof, and the third myogenic regulatory factor polypeptide is a MYOD polypeptide or a fragment thereof).

Embodiment 100. The method of any one of embodiments 92-99, wherein the Activin A inhibitor is selected from: A-83-01, E-616542, SB431542, TGFβRI-IN-3, R-268712, Follistatin, and Follistatin-like-3.

Embodiment 101. The method of embodiment 100, wherein the Activin A inhibitor is A-83-01.

Embodiment 102. The method of any one of embodiments 92-101, wherein the BMP inhibitor is selected from: LDN193189, Dorsomorphin, Noggin, Chrodin, and Gremlin.

Embodiment 103. The method of embodiment 102, wherein the BMP inhibitor is LDN193189.

Embodiment 104. The method of any one of embodiments 92-103, wherein the WNT activator is selected from: CHIR99021, BIO, AZD1080, WNT1a, WNT3a, WNT4, and WNT7.

Embodiment 105. The method of embodiment 104, wherein the WNT activator is CHIR99021.

Embodiment 106. The method of any one of embodiments 92-105, further comprising contacting the cell line or immortalized cell line with a culture media comprising a histone deacetylase inhibitor.

Embodiment 107. The method of embodiment 106, wherein the histone deacetylase inhibitor is sodium butyrate.

Embodiment 108. The method of any one of embodiments 92-107, wherein the cell line, the immortalized cell line, or the late passage cell line are from a species selected from: poultry, livestock, game, or aquatic animal species.

Embodiment 109. The method of embodiment 108, wherein the species is Gallus gallus.

Embodiment 110. The method of embodiment 108, wherein the species is Bovine taurus.

Embodiment 111. The method of any one of embodiments 92-110, wherein the cell line, the immortalized cell line, or the late passage cell line is a fibroblast cell line.

Embodiment 112. The method of any one of embodiments 92-111, wherein the cell line, immortalized cell line, or the late passage cell line are not embryonic or induced pluripotent stem cells.

Embodiment 113. The method of any one of embodiments 94-112, wherein the late passage cell line has exceeded 60 population doublings.

Embodiment 114. The method of any one of embodiments 94-113, wherein the control late passage cell line has lost myogenic differentiation capacity at or above 60 population doublings.

Embodiment 115. The method of any one of embodiments 92-114, wherein prior to exposing the cell line, immortalized cell line, or the late passage cell line to the methods of any one of embodiments 1-24, the cell line, the immortalized cell line, or the late passage cell line comprises a population doubling level (PDL) of at least 60.

Embodiment 116. The method of any one of embodiments 92-115, wherein the method results in the cell line, the immortalized cell line, or the late passage cell line exhibiting increased Pax7 expression, increased MyHC1 expression, and/or increased myotube formation, as compared to a cell line or immortalized cell line that are not exposed to the at least first Activin A inhibitor, at least first BMP inhibitor, at least first WNT activator, one or more myogenic regulatory factors, epigenetic modulator, or a combination thereof.

Embodiment 117. The method of any one of embodiments 92-116, wherein the method results in the cell line, the immortalized cell line, or the late passage cell line exhibiting an improved myogenic differentiation capacity after at least 60 passages compared with a cell line, an immortalized cell line, or a late passage cell line not exposed to the at least first Activin A inhibitor, at least first BMP inhibitor, at least first WNT activator, one or more myogenic regulatory factors, epigenetic modulator, or a combination thereof.

Embodiment 118. The method of any one of embodiments 92-117, further comprising the step of adapting the cell line for suspension culture.

Embodiment 119. The method of any one of embodiments 92-118, wherein inducing myogenic-specific differentiation comprises contacting the cell line or immortalized cell line with a differentiation medium.

Embodiment 120. A population of cells produced by any of the methods of embodiments 92-119.

Embodiment 121. A population of myocytes, myoblasts, myotubes, multinucleated myotubes, satellite cells, skeletal muscle fibers, or any combination thereof produced by the methods of any one of embodiments 92-119.

Embodiment 122. An in vitro method for producing a cell-based meat product, comprising:

forming the myocytes, myoblasts, myotubes, or a combination thereof, of embodiments 92-119, into a cell based meat product.

### 9. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

### SEQUENCE APPENDIX

| SEQ ID | |
|---|---|
| NO: | Sequence |
| SEQ ID | ATGGACTTACTGGGCCCCATGGAAATGACGGAGGGCTCCCTCTGCTCC |
| NO: 43 | TTCACGGCCGCCGATGACTTCTATGACGACCCGTGCTTCAACACGTCG |
| (MyoD | GACATGCACTTCTTCGAGGACCTGGACCCCCGGCTGGTGCACGTGGGC |
| mRNA | GGGCTGCTGAAAGCCGAGGAGCACCCGCACCACCACGGGCACCACCA |
| sequence) | CGGGAACCCACACGAGGAGGAGCACGTGCGGGCGCCCAGTGGGCACC |
| | ACCAGGCCGGCCGCTGCCTGCTGTGGGCGTGCAAGGCCTGCAAGAGGA |
| | AGACCACCAACGCTGACCGCCGCAAAGCCGCCACCATGAGGGAACGG |
| | CGGCGGCTCAGCAAGGTCAACGAGGCCTTTGAGACCCTCAAGCGCTGC |
| | ACTTCCACCAACCCCAACCAGCGCCTGCCCAAGGTGGAGATCCTGCGC |
| | AACGCCATCCGCTACATCGAGAGCCTGCAGGCCCTGCTGCGTGAGCAG |
| | GAGGATGCATACTACCCAGTGCTGGAGCACTACAGCGGGGAGTCAGAT |
| | GCCTCCAGCCCTCGCTCCAACTGCTCCGACGGCATGATGGAGTACAGC |
| | GGGCCGCCCTGTAGCTCTCGCAGGAGAAACAGCTACGACAGCAGCTAC |
| | TACACGGAATCACCAAATGACCCAAAGCATGGGAAGAGTTCTGTTGTT |
| | TCCAGCCTCGACTGCCTCTCAAGCATTGTGGAGAGGATTTCCACAGAC |
| | AACTCCACATGTCCCATACTGCCTCCAGCTGAAGCTGTAGCTGAAGGG |
| | AGTCCCTGTTCCCCCCAGGAAGGAGGAAACCTGAGTGACAGTGGAGCC |
| | CAGATTCCTTCCCCCACCAACTGCACCCCTCTTCCCCAGGAAAGCAGC |
| | AGCAGCAGCAGCAGCAATCCAATCTACCAAGTGCTATAAAAAAAAAA |
| | AAAAAAAAAAAAAAAAAAA |
| SEQ ID | GCCCCTGCAGCCGAATTATATTATTTTTGCCAAATAATTTTTAACAAAA |
| NO: 44 | GCTCTGAAGTCTTCTTCATTTAAATTCTTAGATGATACTTCATCTGGAA |
| (FC551- | AATTGTCCCAATTAGTAGCATCACGCTGTGAGTAAGTTCTAAACCATTT |
| ggMyoD) | TTTTATTGTTGTATTATCTCTAATCTTACTACTCGATGAGTTTTCGGTAT |
| | TATCTCTATTTTTAACTTGGAGCAGGTTCCATTCATTGTTTTTTTCATCA |
| | TAGTGAATAAAATCAACTGCTTTAACACTTGTGCCTGAACACCATATCC |
| | ATCCGGCGTAATACGACTCACTATAGGGAGAGCGGCCGCGTCGACATG |
| | CCCGCCGTGACCGTCGAGAACCCGCTGACGCTGCCCCGCGTATCCGCA |
| | CCCGCCGACGCCGTCGCACGTCCCGTGCTCACCGTGACCACCGCGCCC |
| | AGCGGTTTCGAGGGCGAGGGCTTCCCGGTGCGCCGCGCGTTCGCCGGG |
| | ATCAACTACCGCCACCTCGACCCGTTCATCATGATGGACCAGATGGGT |
| | GAGGTGGAGTACGCGCCCGGGGAGCCCAAGGGCACGCCCTGGCACCC |
| | GCACCGCGGCTTCGAGACCGTGACCTACATCGTCGACGCGGCCGCCAG |
| | ATCTTCCGGATGGCTCGAGTTTTTCAGCAAGATATGATAAGATACATTG |
| | ATGAGTTTGGACAAACCACAACTAGAATGCAGTGAAAAAAATGCTTTA |
| | TTTGTGAAATTTGTGATGCTATTGCTTTATTTGTAACCATTATAAGCTG |
| | CAATAAACAAGTTTAGTTAACGCATGATACAAAGGCATTAAAGCAGCG |
| | TATCCACATAGCGTAAAAGGAGCAACATAGTTAAGAATACCAGTCAAT |
| | CTTTCACAAATTTTGTAATCCAGAGGTTGATTTCAGGCACCGGGCTTGC |
| | GGGTCATGCACCAGGTGCGCGGTCCTTCGGGCACCTCGACGTCGGCGG |
| | TGACGGTGAAGCCGAGCCGCTCGTAGAAGGGGAGGTTGCGGGGCGCG |
| | GAGGTCTCCAGGAAGGCGGGCACCCCGGCGCGCTCGGCCGCCTCCACT |
| | CCGGGGAGCACGACGGCGCTGCCCAGACCCTTGCCCTGGTGGTCGGGC |
| | GAGACGCCGACGGTGGCCAGGAACCACGCGGGCTCCTTGGGCCGGTG |
| | CGGCGCCAGGAGGCCTTCCATCTGTTGCTGCGCGGCCAGCCGGGAACC |
| | GCTCAACTCGGCCATGCGCGGGCCGATCTCGGCGAACACCGCCCCCGC |
| | TTCGACGCTCTCCGGCGTGGTCCAGACCGCCACCGCGGCGCCGTCGTC |
| | CGCGACCCACACCTTGCCGATGTCGAGCCCGACGCGCGTGAGGAAGAG |
| | TTCTTGCAGCTCGGTGACCCGCTCGATGTGGCGGTCCGGATCGACGGT |
| | GTGGCGCGTGGCGGGGTAGTCGGCGAACGCGGCGGCGAGGGTGCGTA |
| | CGGCCCTGGGGACGTCGTCGCGGGTGGCGAGGCGCACCGTGGGCTTGT |
| | ACTCGGTCATAGGGCCGGGATTCTCCTCCACGTCACCGCATGTTAGAA |
| | GACTTCCTCTGCCCTCGCGAGATCCGGTGGAGCCGGGTCCGGCGGTGC |
| | CGTCCACGGCAGAATTGGACGACTGAGCGCGGGATCTGGCGAAGGCG |
| | ATGGGGGTCTTGAAGGCGTGCTGGTACTCCACGATGCCCAGCTCGGTG |
| | TTGCTGTGCAGCTCCTCCACGCGGCGGAAGGCGAACATGGGGCCCCCG |
| | TTCTGCAGGATGCTGGGGTGGATGGCGCTCTTGAAGTGCATGTGGCTG |
| | TCCACCACGAAGCTGTAGTAGCCGCCGTCGCGCAGGCTGAAGGTGCGG |
| | GCGAAGCTGCCCACCAGCACGTTATCGCCCATGGGGTGCAGGTGCTCC |
| | ACGGTGGCGTTGCTGCGGATGATCTTGTCGGTGAAGATCACGCTGTCC |
| | TCGGGGAAGCCGGTGCCCACCACCTTGAAGTCGCCGATCACGCGGCCG |
| | GCCTCGTAGCGGTAGCTGAAGCTCACGTGCAGCACGCCGCCGTCCTCG |
| | TACTTCTCGATGCGGGTGTTGGTGTAGCCGCCGTTGTTGATGGCGTGCA |
| | GGAAGGGGTTCTCGTAGCCGCTGGGGTAGGTGCCGAAGTGGTAGAAG |
| | CCGTAGCCCATCACGTGGCTCAGCAGGTAGGGGCTGAAGGTCAGGGCG |
| | CCTTTGGTGCTCTTCATCTTGTTGGTCATGCGGCCCTGCTTGGGGGTGC |
| | CCTCTCCGCCGCCCACCAGCTCGAACTCCACGCCGTTCAGGGTGCCGG |
| | TGATGCGGCACTCGATCTCCATGGCGGGCAGGCCGCTCTCGTCGCTCTC |
| | CATGGTGGCGTCTAGCGTAGGCGCCGGTCACAGCTTGGATCTGTAACG |
| | GCGCAGAACAGAAAACGAAACAAAGACGTAGAGTTGAGCAAGCAGGG |
| | TCAGGCAAAGCGTGGAGAGCCGGCTGAGTCTAGGTAGGCTCCAAGGG |
| | AGCGCCGGACAAAGGCCCGGTCTCGACCTGAGCTTTAAACTTACCTAG |
| | ACGGCGGACGCAGTTCAGGAGGCACCACAGGCGGGAGGCGGCAGAAC |
| | GCGACTCAACCGGCGTGGATGGCGGCCTCAGGTAGGGCGGCGGGCGC |
| | GTGAAGGAGAGATGCGAGCCCCTCGAAGCTTCAGCTGTGTTCTGGCGG |
| | CAAACCCGTTGCGAAAAAGAACGTTCACGGCGACTACTGCACTTATAT |
| | ACGGTTCTCCCCCACCCTCGGGAAAAAGGCGGAGCCAGTACACGACAT |
| | CACTTTCCCAGTTTACCCCGCGCCACCTTCTCTAGGCACCCGTTCAATT |
| | GCCGACCCCTCCCCCCAACTTCTCGGGGACTGTGGGCGATGTGCGCTCT |
| | GCCCACTGACGGGCACCGGAGCGATCGCAGATCCTTATCTTTCTAGAA |
| | ATTCTACCGGGTAGGGGAGGCGCTTTTCCCAAGGCAGTCTGGAGCATG |
| | CGCTTTAGCAGCCCCGCTGGCACTTGGCGCTACACAAGTGGCCTCTGG |
| | CCTCGCACACATTCCACATCCACCGGTAGCGCCAACCGGCTCCGTTCTT |
| | TGGTGGCCCCTTCGCGCCACCTTCTACTCCTCCCCTAGTCAGGAAGTTC |
| | CCCCCCGCCCCGCAGCTCGCGTCGTGCAGGACGTGACAAATGGAAGTA |
| | GCACGTCTCACTAGTCTCGTGCAGATGGACAGCACCGCTGAGCAATGG |
| | AAGCGGGTAGGCCTTTGGGGCAGCGGCCAATAGCAGCTTTGCTCCTTC |
| | GCTTTCTGGGCTCAGCAGCTGGGAAGGGTGGGTCCGGGGGCGGGCTCA |
| | GGGGCGGGCTCAGGGGCGGGGCGGGCGCCCGAAGGTCCTCCGGAGGC |
| | CCGGCATTCTGCACGCTTCAAAAGCGCACGTCTGCCGCGCTGTTCTCCT |
| | CTTCCTCATCTCCGGGCCTTTCGACCTGGATCCGCCACCATGGACTTAC |
| | TGGGCCCCATGGAAATGACGGAGGGCTCCCTCTGCTCCTTCACGGCCG |
| | CCGATGACTTCTATGACGACCCGTGCTTCAACACGTCGGACATGCACTT |
| | CTTCGAGGACCTGGACCCCCGGCTGGTGCACGTGGGCGGGCTGCTGAA |
| | AGCCGAGGAGCACCCGCACCACCACGGGCACCACCACGGGAACCCAC |
| | ACGAGGAGGAGCACGTGCGGGCGCCCAGTGGGCACCACCAGGCCGGC |
| | CGCTGCCTGCTGTGGGCGTGCAAGGCCTGCAAGAGGAAGACCACCAAC |
| | GCTGACCGCCGCAAAGCCGCCACCATGAGGGAACGGCGGCGGCTCAG |
| | CAAGGTCAACGAGGCCTTTGAGACCCTCAAGCGCTGCACTTCCACCAA |
| | CCCCAACCAGCGCCTGCCCAAGGTGGAGATCCTGCGCAACGCCATCCG |
| | CTACATCGAGAGCCTGCAGGCCCTGCTGCGTGAGCAGGAGGATGCATA |
| | CTACCCAGTGCTGGAGCACTACAGCGGGGAGTCAGATGCCTCCAGCCC |
| | TCGCTCCAACTGCTCCGACGGCATGATGGAGTACAGCGGGCCGCCCTG |
| | TAGCTCTCGCAGGAGAAACAGCTACGACAGCAGCTACTACACGGAATC |
| | ACCAAATGACCCAAAGCATGGGAAGAGTTCTGTTGTTTCCAGCCTCGA |
| | CTGCCTCTCAAGCATTGTGGAGAGGATTTCCACAGACAACTCCACATG |
| | TCCCATACTGCCTCCAGCTGAAGCTGTAGCTGAAGGGAGTCCCTGTTCC |
| | CCCCAGGAAGGAGGAAACCTGAGTGACAGTGGAGCCCAGATTCCTTCC |
| | CCCACCAACTGCACCCCTCTTCCCCAGGAAAGCAGCAGCAGCAGCAGC |
| | AGCAATCCAATCTACCAAGTGCTATAAGCTAGCATCGATCAGACATGA |
| | TAAGATACATTGATGAGTTTGGACAAACCACAACTAGAATGCAGTGAA |
| | AAAAATGCTTTATTTGTGAAATTTGTGATGCTATTGCTTTATTTGTAAC |
| | CATTATAAGCTGCAATAAACAAGTTAACAACAACAATTGCATTCATTT |
| | TATGTTTCAGGTTCAGGGGGAGGTGTGGGAGGTTTTTTGTTTAAACCTC |
| | CTGTGTGAAATTATTATCCGCTCATAATTCCACACATTATACGAGCCGG |
| | AAGCATAAAGTGTAAAGCCTGGGGTGCCTAATGAGTGAGCTAACTCAC |
| | ATTAATTGCGTTGCGCTCACTGCCAATTGCTTTCCAGTCGGGAAACCTG |
| | TCGTGCCAGCTGCATTAATGAATCGGCCAACGCGCGGGGAGAGGCGGT |
| | TTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAA |
| | TACGGTTATCCACAGAATCAGGGGATAACGCAGGAAAGAACATGTGA |
| | GCAAAAGGCCAGCAAAAGGCCAGGAACCGTAAAAAGGCCGCGTTGCT |
| | GGCGTTTTTCCATAGGCTCCGCCCCCCTGACGAGCATCACAAAAATCG |
| | ACGCTCAAGTCAGAGGTGGCGAAACCCGACAGGACTATAAAGATACC |
| | AGGCGTTTCCCCCTGGAAGCTCCCTCGTGCGCTCTCCTGTTCCGACCCT |
| | GCCGCTTACCGGATACCTGTCCGCCTTTCTCCCTTCGGGAAGCGTGGCG |
| | CTTTCTCATAGCTCACGCTGTAGGTATCTCAGTTCGGTGTAGGTCGTTC |
| | GCTCCAAGCTGGGCTGTGTGCACGAACCCCCCGTTCAGCCCGACCGCT |
| | GCGCCTTATCCGGTAACTATCGTCTTGAGTCCAACCCGGTAAGACACG |
| | ACTTATCGCCACTGGCAGCAGCCACTGGTAACAGGATTAGCAGAGCGA |
| | GGTATGTAGGCGGTGCTACAGAGTTCTTGAAGTGGTGGCCTAACTACG |
| | GCTACACTAGAAGAACAGTATTTGGTATCTGCGCTCTGCTGAAGCCAG |
| | TTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAG |
| | AAAAAAAGGATCTCAAGAAGATCCTTTGATCTTTTCTACGGGGTCTGA |
| | CGCTCAGTGGAACGAAAACTCACGTTAAGGGATTTTGGTCATGAGATT |
| | ATCAAAAAGGATCTTCACCTAGATCCTTTTAAATTAAAAATGAAGTTTT |
| | AAATCAATCTAAAGTATATATGAGTAAACTTGGTCTGACAGTTACCAA |
| | TGCTTAATCAGTGAGGCACCTATCTCAGCGATCTGTCTATTTCGTTCAT |
| | CCATAGTTGCCTGACTCCCCGTCGTGTAGATAACTACGATACGGGAGG |
| | GCTTACCATCTGGCCCCAGTGCTGCAATGATACCGCGAGACCCACGCT |
| | CACCGGCTCCAGATTTATCAGCAATAAACCAGCCAGCCGGAAGGGCCG |
| | AGCGCAGAAGTGGTCCTGCAACTTTATCCGCCTCCATCCAGTCTATTAA |
| | TTGTTGCCGGGAAGCTAGAGTAAGTAGTTCGCCAGTTAATAGTTTGCG |
| | CAACGTTGTTGCCATTGCTACAGGCATCGTGGTGTCACGCTCGTCGTTT |
| | GGTATGGCTTCATTCAGCTCCGGTTCCCAACGATCAAGGCGAGTTACA |
| | TGATCCCCCATGTTGTGCAAAAAAGCGGTTAGCTCCTTCGGTCCTCCGA |
| | TCGTTGTCAGAAGTAAGTTGGCCGCAGTGTTATCACTCATGGTTATGGC |
| | AGCACTGCATAATTCTCTTACTGTCATGCCATCCGTAAGATGCTTTTCT |
| | GTGACTGGTGAGTACTCAACCAAGTCATTCTGAGAATAGTGTATGCGG |
| | CGACCGAGTTGCTCTTGCCCGGCGTCAATACGGGATAATACCGCGCCA |
| | CATAGCAGAACTTTAAAAGTGCTCATCATTGGAAAACGTTCTTCGGGG |
| | CGAAAACTCTCAAGGATCTTACCGCTGTTGAGATCCAGTTCGATGTAA |
| | CCCACTCGTGCACCCAACTGATCTTCAGCATCTTTTACTTTCACCAGCG |
| | TTTCTGGGTGAGCAAAAACAGGAAGGCAAAATGCCGCAAAAAAGGGA |
| | ATAAGGGCGACACGGAAATGTTGAATACTCATACTCTTCCTTTTTCAAT |
| | ATTATTGAAGCATTTATCAGGGTTATTGTCTCATGAGCGGATACATATT |
| | TGAATGTATTTAGAAAAATAAACAAATAGGGGTTCCGCGCACATTTCC |
| | CCGAAAAGTGCCACCTGACGTCTAAGAAACCATTATTATCATGACATT |
| | AACCTATAAAAATAGGCGTATCACGAGGCC |
| SEQ ID | TGGCACGACAGGTTTCCCGACTGGAAAGCGGGCAGTGAGCGCAACGC |
| NO: 45 | AATTAATGTGAGTTAGCTCACTCATTAGGCACCCCAGGCTTTACACTTT |
| (pFS2- | ATGCTTCCGGCTCGTATGTTGTGTGGAATTGTGAGCGGATAACAATTTC |
| MCS- | ACACAGGAAACAGCTATGACCATGATTACGCCAAGCTTAAGCTTGCAT |
| mmPGK- | GCCTGCAGGTCGACCTGCAGCGATCGAACTTGTTTATTGCAGCTTATAA |
| Neo_- | TGGTTACAAATAAAGCAATAGCATCACAAATTTCACAAATAAAGCATT |
| EF1_Pax7_ | TTTTTCACTGCATTCTAGTTGTGGTTTGTCCAAACTCATCAATGTATCTT |
| MEF2b_ | ATCATGTCTGGATCCCTTCTGAGGGCAGAGGAAGTCTTAATTCAGAAG |
| MyoD) | AACTCGTCAAGAAGGCGATAGAAGGCGATGCGCTGCGAATCGGGAGC |
| | GGCGATACCGTAAAGCACGAGGAAGCGGTCAGCCCATTCGCCGCCAA |
| | GCTCTTCAGCAATATCACGGGTAGCCAACGCTATGTCCTGATAGCGGT |
| | CCGCCACACCCAGCCGGCCACAGTCGATGAATCCAGAAAAGCGGCCAT |
| | TTTCCACCATGATATTCGGCAAGCAGGCATCGCCATGGGTCACGACGA |
| | GATCCTCGCCGTCGGGCATGCTCGCCTTGAGCCTGGCGAACAGTTCGG |
| | CTGGCGCGAGCCCCTGATGCTCTTCGTCCAGATCATCCTGATCGACAA |
| | GACCGGCTTCCATCCGAGTACGTGCTCGCTCGATGCGATGTTTCGCTTG |
| | GTGGTCGAATGGGCAGGTAGCCGGATCAAGCGTATGCAGCCGCCGCAT |
| | TGCATCAGCCATGATGGATACTTTCTCGGCAGGAGCAAGGTGAGATGA |
| | CAGGAGATCCTGCCCCGGCACTTCGCCCAATAGCAGCCAGTCCCTTCC |
| | CGCTTCAGTGACAACGTCGAGCACAGCTGCGCAAGGAACGCCCGTCGT |
| | GGCCAGCCACGATAGCCGCGCTGCCTCGTCTTGCAGTTCATTCAGGGC |
| | ACCGGACAGGTCGGTCTTGACAAAAAGAACCGGGCGCCCCTGCGCTGA |
| | CAGCCGGAACACGGCGGCATCAGAGCAGCCGATTGTCTGTTGTGCCCA |
| | GTCATAGCCGAATAGCCTCTCCACCCAAGCGGCCGGAGAACCTGCGTG |
| | CAATCCATCTTGTTCAATCATGCCCATGGTGGCGGCACGTGAGGTAGG |
| | TCGAAAGGCCCGGAGATGAGGAAGAGGAGAACAGCGCGGCAGACGTG |
| | CGCTTTTGAAGCGTGCAGAATGCCGGGCCTCCGGAGGACCTTCGGGCG |
| | CCCGCCCCGCCCCTGAGCCCGCCCCTGAGCCCGCCCCCGGACCCACCC |
| | TTCCCAGCTGCTGAGCCCAGAAAGCGAAGGAGCAAAGCTGCTATTGGC |
| | CGCTGCCCCAAAGGCCTACCCGCTTCCATTGCTCAGCGGTGCTGTCCAT |
| | CTGCACGAGACTAGTGAGACGTGCTACTTCCATTTGTCACGTCCTGCAC |
| | GACGCGAGCTGCGGGGCGGGGGGGAACTTCCTGACTAGGGGAGGAGT |
| | AGAAGGTGGCGCGAAGGGGCCACCAAAGAACGGAGCCGGTTGGCGCT |
| | ACCGGTGGATGTGGAATGTGTGCGAGGCCAGAGGCCACTTGTGTAGCG |
| | CCAAGTGCCAGCGGGGCTGCTAAAGCGCATGCTCCAGACTGCCTTGGG |
| | AAAAGCGCCTCCCCTACCCGGTAGAATTGGTGGCGATGCCGAGCTCGG |
| | TACCAAGCTTAAGTTTAAACGCTAGCAGTAGAATTCGGATCCTACCGT |
| | TCGTATAGCATACATTATACGAAGTTATGTCGACGATGTAGGTCACGG |
| | TCTCGAAGCCGCGGTGCGGGTGCCAGGGCGTGCCCTTGGGCTCCCCGG |
| | GCGCGTACTCCACCTCACCCATCTGGTCCATCATGATGAACGGTCGAG |
| | GTGGCGGTAGTTGATCCCGGCGAACGCGCGGCGCACCGGGAAGCCCTC |
| | GCCCTCGAAACCGCTGGGCGCGGTGGTCACGGTGAGCACGGGACGTGC |
| | GACGGCGTCGGCGGGTGCGGATACGCGGGGCAGCGTCAGCGGGTTCTC |
| | GACGGTCACGGCGGGCATGTCGACGGCGCGCCGATATCGTTAACTCTA |
| | GAGCCATGAGTACACCCTGCTGGAATCACGAGGGCTTTCACTGCTGCT |
| | GCCTCGTGGCTGCTCGCCGCCCCGGGTTCAGAAGCGAAAGGCTGGGGC |
| | TGCCAGCACCTTCGGTGGCTTTCGTCAGCACCCGTCAGCCGGGAACAT |
| | GTGGCGTGGGGGCACTGCGCGCAGTTTGGAGCTCTCAAGCAGAAGCCA |
| | CGAATCCTTGCCGCTGAAGCGATTCTCTCTGGCCGCATCTGAAACGCC |
| | AGGCCAGAGGAGGTCTGTGCAAGTAACTACTCCCGGAGCTCGCTGACC |
| | GCCTCCCCTCGGGCCGCAGCCGCGGGTCAGAGGGGCAGAGCTCGGGG |
| | ACTCTACGGCTGCCGTGCTGCGCGGGAGGCGGCGGAGCGGCAACGCC |
| | GTCCCGCCCTGCAGTGCTCCGGGCGCTGCCGTCCGGCCTGGGGGCGGC |
| | GAGCCCCTCCCGGCAGTCGCTCGTGACTCCCGCCCGATCCGGCCGCCG |
| | CGGGGCTCTGCTCCTCAGCCCCGCCCCTCCCCGCCGCTCCTCAGCGGAG |
| | CGCCGCGTGCCGCGCTGCCGGGCGGGGCGTTGCGCGGGGGCCGCCGG |
| | GAGCGGTGACGCGTCGCCGAGGCCCCGCCCCGTCGCAGAGTATATAAG |
| | GGGCGGCGCTCACGCCGTCGTTCTTTTCCGCTCCGGGTTTGCCGACGCC |
| | GTGCGGGTGAGGCGCGTGCGGCGGGGCAGGCCGGGCCCGGGGGTGGG |
| | GGGTGCGGGGTGCGGGGATGCTCGGGGGGAGCCTCGTTGCACCGTGA |
| | GCGCGGGGCCGGGGCGCGGCCGCGGAGAGGCGCGTGGGAACGGCGGA |
| | GTGGGGAGGGGGAAGCGAGATGAGGGACGGGTGGGAGAGAGCGGGG |
| | CGAGCGCCGGGGTAAAGCCGACGTGTCCCGCCTGAGGACCCACGCGG |
| | GGCAGCGCGGCGATGAGCACGCGGGGCCCCACCGCCGCCTTGGCTTCG |
| | CGCGGAGCCGCCCGGCGCTGGGGCGCGGCGTACGGTGCTCGGCCTCGG |
| | GCGGGTGGCGGCCGGGGACCCCGGGGCCGCGCCGTCGGTGCCGCGGT |
| | GGGGGAGGGGAGGCGAGGCCGGAGCACGAGAAAAAAAATGGCCGCC |
| | CGCTCTTCCTGCCGCCGCCAGCTCACAAAATGGAGGACGGCGGCCCGG |
| | GCGGGGTGAGTCACACACAAAGGAGAGGGGCGGCCCCGCCCTCCGCG |
| | CCGCCCGTGGCCGCGCCGCCGCCCCGCGCCCCCACCCCAGCCCCTGCC |
| | CGGCCCGGCCGCCCTGCGGGGAGGGGGCGACGGCGGAGGCGCGCTGT |
| | AGGGGACCCCGGGCCACCGCCCGCCCCTCCGCTCCCCTCCCCCCTCCTC |
| | CCCCCCCTGGCGGGGCGGGGCCGGGGCTGCACGGGCCCTCTGCCCCCT |
| | CGGCCGGCGCCCGGCGGGCGGCGCTGCTGCGGGACGGGGGCGGCCGC |
| | AGTAACCGGGATTCGTTCTGGAACTTTCAGGTGTCGTTTCTCTTTGGCC |
| | GGAAGAAAGAAGCTAAAGACCATCCGAATCGCGACGCCACCATGGCA |
| | GCGCTCCCCGGGACGGTACCGCGGATGATGCGCCCGGCGCCGGGGCA |
| | GAACTACCCGCGCACCGGCTTCCCTTTGGAAGTGTCCACCCCGCTGGG |
| | CCAGGGCCGGGTGAACCAACTCGGAGGGGTTTTCATCAACGGGCGCCC |
| | ACTGCCCAACCACATCCGCCATAAGATCGTGGAGATGGCTCACCACGG |
| | CATCCGGCCCTGCGTGATCTCCAGGCAGCTGAGGGTCTCCCACGGCTG |
| | CGTTTCCAAAATCCTCTGCAGGTACCAAGAGACGGGCTCCATCCGGCC |
| | CGGGGCCATCGGGGGCAGCAAGCCCAGGCAGGTTGCGACTCCCGACG |
| | TGGAGAAGAAAATCGAGGAATACAAGAGGGAGAACCCTGGGATGTTC |
| | AGCTGGGAGATCCGGGACAGGCTGCTGAAGGACGGACACTGCGACCG |
| | CAGCACTGTGCCCTCAGTGAGTTCGATTAGCCGTGTGCTACGCATCAA |
| | ATTCGGGAAGAAAGAGGAAGAGGAGGACTGCGACAAGAAGGAGGAA |
| | GACGGGGAGAAGAAGGCCAAGCACAGCATAGATGGCATCCTGGGCGA |
| | CAAAGGGAACAGGCTGGATGAAGGCTCCGATGTCGAATCAGAACCAG |
| | ACCTGCCTTTGAAGAGGAAGCAGCGCCGCAGCCGGACCACTTTCACTG |
| | CCGAGCAGCTGGAGGAGCTGGAGAAGGCCTTTGAGAGGACCCACTAC |
| | CCGGACATCTACACCAGGGAGGAGCTGGCACAGAGAACCAAGCTCAC |
| | CGAGGCCCGTGTTCAGGTGTGGTTCAGCAACCGACGAGCAAGATGGCG |
| | CAAGCAGGCGGGTGCAAACCAACTCGCAGCATTCAACCATCTGCTGCC |
| | AGGGGGATTCCCACCCACGGGAATGCCAACTCTGCCCCCGTACCAGCT |
| | GCCAGACTCCACCTACCCAACCACCACCATTTCCCAAGATGGAGGCAG |
| | CACCGTGCACAGACCCCAGCCCTTGCCACCATCCACCATGCACCAGGG |
| | AGGGCTCGCTGCCGCCGCTGCAGCCGACTCCAGCTCTGCCTATGGGGC |
| | CCGACACAGCTTCTCCAGCTACTCAGACAGCTTCATGAATGCTGCAGC |
| | TCCTGCCAACCACATGAATCCTGTTAGCAATGGCCTCTCTCCGCAGAA |
| | GCAGGGTGCCCAAAACAAGATGCAGTGCTCCAGGTGGAACCTCACCAT |
| | AGCCTTGAACAATCAGGTGATGAGCATCCTGAGCAACCCCAGCGGGGT |
| | TCCTCCGCAGCCCCAGGCTGACTTCTCCATCTCTCCTCTTCACGGTGGC |
| | CTGGACACCACCAACTCCATCTCTGCCAGCTGCAGCCAGCGGAGTGAC |
| | TCCATCAAGTCCGTGGACAGCCTCCCGACCTCGCAGTCCTACTGTCCTC |
| | CCACCTACAGCACCACCAGTTACAGCGTGGACCCGGTGGCTGGCTACC |
| | AGTATGGGCAGTATGGACAAACTGCTGTTGATTATTTGACCAAGAACG |
| | TGAGCCTGTCCACGCAGCGCAGGATGAAGCTGGGAGAACATTCGGCCG |
| | TTCTGGGGCTCCTACCAGTAGAGACAGGCCAAGCTTACGCCACAAACT |
| | TCTCTCTGCTAAAGCAAGCAGGTGATGTTGAAGAAAACCCCGGGCCTG |
| | GCCGGAAAAAAATCCAGATCAGCCGAATATTGGATCAGCGGAACCGG |
| | CAGGTGACCTTCACCAAGCGGAAATTCGGGCTGATGAAGAAGGCGTAT |
| | GAGCTGAGCGTGCTGTGCGACTGCGAGATCGCCCTCATCATCTTCAAC |
| | AGCACCAACCGCCTCTTCCAGTACGCCAGCACCGACATGGACAAAGTG |
| | CTGCTCAAGTACACGGAGTACAGCGAACCCCACGAGAGCCGCACCAA |
| | CTCCGACATCCTCGAGACGCTGAAGCGCAAAGGGTTGGGGCTGGAGA |
| | GCCACGAGCTGGAGCTGGAAGAGGGGCTGGATCCTGGTGAGAAGCTG |
| | CGGCAGATGAATGAGGGCATGGATCTGACGGTGGCACGGCCCCGCTTT |
| | TACAGCCCGGTGCCGCTGCCCGAGGTCCCCTATGGCAGCTCCCCGTCG |
| | AGCAGCAGTGATGGAGCCCTGGGCAGCGCCAGCAGCTCCCCGCAGAG |
| | CCAGGGCCGCCCGCCCGCCTTCAAACCCACAGCGCCGAAGCCATCGGG |
| | ACGCTCACCAGGACCGATGCCCCCAGGTATTGGCTACCCCCTGTTCCCC |
| | GCTGGCAGCCTGAACCGAGCCTTGGCAACCAAGACCCCACCACCGCTG |
| | TACCTGGGAGCCGATGGGCAGCGCCGGAGCGGAGGCAGCACTGCACG |
| | GCCGCTGTACTCTGGTCTGCAGACCTTGAACCCAGTGCTCGCCTCAGGC |
| | AGCGCCAGCATTCCCAGCCACAGCCTCACTGGTTTCCCCTTCCTCGCTC |
| | CGGCACAAGCAGCAGAGTTCGGGGCTGGCGAGGCCCCACCACCTCCTG |
| | GCTTCCTGCAGCCTGGCCCTCCAGCTCCATGGCAGCCCCCACGGGACA |
| | TGGCAGCACTAGGGGCCAGCAGCAGGATCGTCCCCACCGAGGACCTG |
| | GCCCCCGGCAGCTCCCCGCAGCCCCACGCCATCAGCATCAAGTCAGAG |
| | CGGGTGTCCCCAGGGCTCGGCTGCCCCTCAGGTGCCCCACAGCCCTCT |
| | CAGGGCAGCCTGACCTCCCTGAGCGAAGCCCCCCAAGGCGCTGCAGAC |
| | CTCCAGCCACGGGATGACTACTCCAAGGGCTACCCCTACCCCCCGCCC |
| | CCGCCCCGGCCGCTGGCGGAGGAGCAGAGGGCCACTGCCGCCGTCCCG |
| | GTCCCCGCGCGGCGGGCGCAGGCTGAGGATGCGTGGCAGAGAGAGGG |
| | CAGGGGAAGTCTTCTAACATGCGGGGACGTGGAGGAAAATCCCGGCC |
| | CAGACTTACTGGGCCCCATGGAAATGACGGAGGGCTCCCTCTGCTCCT |
| | TCACGGCCGCCGATGACTTCTATGACGACCCGTGCTTCAACACGTCGG |
| | ACATGCACTTCTTCGAGGACCTGGACCCCCGGCTGGTGCACGTGGGCG |
| | GGCTGCTGAAAGCCGAGGAGCACCCGCACCACCACGGGCACCACCAC |
| | GGGAACCCACACGAGGAGGAGCACGTGCGGGCGCCCAGTGGGCACCA |
| | CCAGGCCGGCCGCTGCCTGCTGTGGGCGTGCAAGGCCTGCAAGAGGAA |
| | GACCACCAACGCTGACCGCCGCAAAGCCGCCACCATGAGGGAACGGC |
| | GGCGGCTCAGCAAGGTCAACGAGGCCTTTGAGACCCTCAAGCGCTGCA |
| | CTTCCACCAACCCCAACCAGCGCCTGCCCAAGGTGGAGATCCTGCGCA |
| | ACGCCATCCGCTACATCGAGAGCCTGCAGGCCCTGCTGCGTGAGCAGG |
| | AGGATGCATACTACCCAGTGCTGGAGCACTACAGCGGGGAGTCAGATG |
| | CCTCCAGCCCTCGCTCCAACTGCTCCGACGGCATGATGGAGTACAGCG |
| | GGCCGCCCTGTAGCTCTCGCAGGAGAAACAGCTACGACAGCAGCTACT |
| | ACACGGAATCACCAAATGACCCAAAGCATGGGAAGAGTTCTGTTGTTT |
| | CCAGCCTCGACTGCCTCTCAAGCATTGTGGAGAGGATTTCCACAGACA |
| | ACTCCACATGTCCCATACTGCCTCCAGCTGAAGCTGTAGCTGAAGGGA |
| | GTCCCTGTTCCCCCCAGGAAGGAGGAAACCTGAGTGACAGTGGAGCCC |
| | AGATTCCTTCCCCCACCAACTGCACCCCTCTTCCCCAGGAAAGCAGCA |
| | GCAGCAGCAGCAGCAATCCAATCTACCAAGTGCTATAATCGAGGTTAA |
| | CGCGGCCGCCACGTGCTCGAGGAGTTGCCAGCCATCTGTTGTTTGCCCC |
| | TCCCCCGTGCCTTCCTTGACCCTGGAAGGTGCCACTCCCACTGTCCTTT |
| | CCTAATAAAATGAGGAAATTGCATCATAACTTCGTATAGCATACATTA |
| | TACGAACGGTAGGATCCCCGGGTACCGAGCTCGAATTCACTGGCCGTC |
| | GTTTTACAACGTCGTGACTGGGAAAACCCTGGCGTTACCCAACTTAAT |
| | CGCCTTGCAGCACATCCCCCTTTCGCCAGCTGGCGTAATAGCGAAGAG |
| | GCCCGCACCGATCGCCCTTCCCAACAGTTGCGCAGCCTGAATGGCGAA |
| | TGGCGCCTGATGCGGTATTTTCTCCTTACGCATCTGTGCGGTATTTCAC |
| | ACCGCATATGGTGCACTCTCAGTACAATCTGCTCTGATGCCGCATAGTT |
| | AAGCCAGCCCCGACACCCGCCAACACCCGCTGACGCGCCCTGACGGGC |
| | TTGTCTGCTCCCGGCATCCGCTTACAGACAAGCTGTGACCGTCTCCGGG |
| | AGCTGCATGTGTCAGAGGTTTTCACCGTCATCACCGAAACGCGCGAGA |
| | CGAAAGGGCCTCGTGATACGCCTATTTTTATAGGTTAATGTCATGATAA |
| | TAATGGTTTCTTAGACGTCAGGTGGCACTTTTCGGGGAAATGTGCGCG |
| | GAACCCCTATTTGTTTATTTTTCTAAATACATTCAAATATGTATCCGCTC |
| | ATGAGACAATAACCCTGATAAATGCTTCAATAATATTGAAAAAGGAAG |
| | AGTATGAGTATTCAACATTTCCGTGTCGCCCTTATTCCCTTTTTTGCGGC |
| | ATTTTGCCTTCCTGTTTTTGCTCACCCAGAAACGCTGGTGAAAGTAAAA |
| | GATGCTGAAGATCAGTTGGGTGCACGAGTGGGTTACATCGAACTGGAT |
| | CTCAACAGCGGTAAGATCCTTGAGAGTTTTCGCCCCGAAGAACGTTTT |
| | CCAATGATGAGCACTTTTAAAGTTCTGCTATGTGGCGCGGTATTATCCC |
| | GTATTGACGCCGGGCAAGAGCAACTCGGTCGCCGCATACACTATTCTC |
| | AGAATGACTTGGTTGAGTACTCACCAGTCACAGAAAAGCATCTTACGG |
| | ATGGCATGACAGTAAGAGAATTATGCAGTGCTGCCATAACCATGAGTG |
| | ATAACACTGCGGCCAACTTACTTCTGACAACGATCGGAGGACCGAAGG |
| | AGCTAACCGCTTTTTTGCACAACATGGGGGATCATGTAACTCGCCTTGA |
| | TCGTTGGGAACCGGAGCTGAATGAAGCCATACCAAACGACGAGCGTG |
| | ACACCACGATGCCTGTAGCAATGGCAACAACGTTGCGCAAACTATTAA |
| | CTGGCGAACTACTTACTCTAGCTTCCCGGCAACAATTAATAGACTGGA |
| | TGGAGGCGGATAAAGTTGCAGGACCACTTCTGCGCTCGGCCCTTCCGG |
| | CTGGCTGGTTTATTGCTGATAAATCTGGAGCCGGTGAGCGTGGCTCTCG |
| | CGGTATCATTGCAGCACTGGGGCCAGATGGTAAGCCCTCCCGTATCGT |
| | AGTTATCTACACGACGGGGAGTCAGGCAACTATGGATGAACGAAATA |
| | GACAGATCGCTGAGATAGGTGCCTCACTGATTAAGCATTGGTAACTGT |
| | CAGACCAAGTTTACTCATATATACTTTAGATTGATTTAAAACTTCATTT |
| | TTAATTTAAAAGGATCTAGGTGAAGATCCTTTTTGATAATCTCATGACC |
| | AAAATCCCTTAACGTGAGTTTTCGTTCCACTGAGCGTCAGACCCCGTAG |
| | AAAAGATCAAAGGATCTTCTTGAGATCCTTTTTTTCTGCGCGTAATCTG |
| | CTGCTTGCAAACAAAAAAACCACCGCTACCAGCGGTGGTTTGTTTGCC |
| | GGATCAAGAGCTACCAACTCTTTTTCCGAAGGTAACTGGCTTCAGCAG |
| | AGCGCAGATACCAAATACTGTTCTTCTAGTGTAGCCGTAGTTAGGCCA |
| | CCACTTCAAGAACTCTGTAGCACCGCCTACATACCTCGCTCTGCTAATC |
| | CTGTTACCAGTGGCTGCTGCCAGTGGCGATAAGTCGTGTCTTACCGGGT |
| | TGGACTCAAGACGATAGTTACCGGATAAGGCGCAGCGGTCGGGCTGA |
| | ACGGGGGGTTCGTGCACACAGCCCAGCTTGGAGCGAACGACCTACACC |
| | GAACTGAGATACCTACAGCGTGAGCTATGAGAAAGCGCCACGCTTCCC |
| | GAAGGGAGAAAGGCGGACAGGTATCCGGTAAGCGGCAGGGTCGGAAC |
| | AGGAGAGCGCACGAGGGAGCTTCCAGGGGGAAACGCCTGGTATCTTT |
| | ATAGTCCTGTCGGGTTTCGCCACCTCTGACTTGAGCGTCGATTTTTGTG |
| | ATGCTCGTCAGGGGGGCGGAGCCTATGGAAAAACGCCAGCAACGCGG |
| | CCTTTTTACGGTTCCTGGCCTTTTGCTGGCCTTTTGCTCACATGTTCTTT |
| | CCTGCGTTATCCCCTGATTCTGTGGATAACCGTATTACCGCCTTTGAGT |
| | GAGCTGATACCGCTCGCCGCAGCCGAACGACCGAGCGCAGCGAGTCA |
| | GTGAGCGAGGAAGCGGAAGAGCGCCCAATACGCAAACCGCCTCTCCC |
| | CGCGCGTTGGCCGATTCATTAATGCAGC |
| SEQ ID | ATGAGTATTCAACATTTCCGTGTCGCCCTTATTCCCTTTTTTGCGGCATT |
| NO: 46 | TTGCCTTCCTGTTTTTGCTCACCCAGAAACGCTGGTGAAAGTAAAAGAT |
| (pFS2- | GCTGAAGATCAGTTGGGTGCACGAGTGGGTTACATCGAACTGGATCTC |
| ggEF1- | AACAGCGGTAAGATCCTTGAGAGTTTTCGCCCCGAAGAACGTTTTCCA |
| ggMyoD- | ATGATGAGCACTTTTAAAGTTCTGCTATGTGGCGCGGTATTATCCCGTA |
| mPGK- | TTGACGCCGGGCAAGAGCAACTCGGTCGCCGCATACACTATTCTCAGA |
| Puro) | ATGACTTGGTTGAGTACTCACCAGTCACAGAAAAGCATCTTACGGATG |
| | GCATGACAGTAAGAGAATTATGCAGTGCTGCCATAACCATGAGTGATA |
| | ACACTGCGGCCAACTTACTTCTGACAACGATCGGAGGACCGAAGGAGC |
| | TAACCGCTTTTTTGCACAACATGGGGGATCATGTAACTCGCCTTGATCG |
| | TTGGGAACCGGAGCTGAATGAAGCCATACCAAACGACGAGCGTGACA |
| | CCACGATGCCTGTAGCAATGGCAACAACGTTGCGCAAACTATTAACTG |
| | GCGAACTACTTACTCTAGCTTCCCGGCAACAATTAATAGACTGGATGG |
| | AGGCGGATAAAGTTGCAGGACCACTTCTGCGCTCGGCCCTTCCGGCTG |
| | GCTGGTTTATTGCTGATAAATCTGGAGCCGGTGAGCGTGGCTCTCGCG |
| | GTATCATTGCAGCACTGGGGCCAGATGGTAAGCCCTCCCGTATCGTAG |
| | TTATCTACACGACGGGGAGTCAGGCAACTATGGATGAACGAAATAGAC |
| | AGATCGCTGAGATAGGTGCCTCACTGATTAAGCATTGGTAACTGTCAG |
| | ACCAAGTTTACTCATATATACTTTAGATTGATTTAAAACTTCATTTTTA |
| | ATTTAAAAGGATCTAGGTGAAGATCCTTTTTGATAATCTCATGACCAA |
| | AATCCCTTAACGTGAGTTTTCGTTCCACTGAGCGTCAGACCCCGTAGAA |
| | AAGATCAAAGGATCTTCTTGAGATCCTTTTTTTCTGCGCGTAATCTGCT |
| | GCTTGCAAACAAAAAAACCACCGCTACCAGCGGTGGTTTGTTTGCCGG |
| | ATCAAGAGCTACCAACTCTTTTTCCGAAGGTAACTGGCTTCAGCAGAG |
| | CGCAGATACCAAATACTGTTCTTCTAGTGTAGCCGTAGTTAGGCCACC |
| | ACTTCAAGAACTCTGTAGCACCGCCTACATACCTCGCTCTGCTAATCCT |
| | GTTACCAGTGGCTGCTGCCAGTGGCGATAAGTCGTGTCTTACCGGGTT |
| | GGACTCAAGACGATAGTTACCGGATAAGGCGCAGCGGTCGGGCTGAA |
| | CGGGGGGTTCGTGCACACAGCCCAGCTTGGAGCGAACGACCTACACCG |
| | AACTGAGATACCTACAGCGTGAGCTATGAGAAAGCGCCACGCTTCCCG |
| | AAGGGAGAAAGGCGGACAGGTATCCGGTAAGCGGCAGGGTCGGAACA |
| | GGAGAGCGCACGAGGGAGCTTCCAGGGGGAAACGCCTGGTATCTTTAT |
| | AGTCCTGTCGGGTTTCGCCACCTCTGACTTGAGCGTCGATTTTTGTGAT |
| | GCTCGTCAGGGGGGCGGAGCCTATGGAAAAACGCCAGCAACGCGGCC |
| | TTTTTACGGTTCCTGGCCTTTTGCTGGCCTTTTGCTCACATGTTCTTTCC |
| | TGCGTTATCCCCTGATTCTGTGGATAACCGTATTACCGCCTTTGAGTGA |
| | GCTGATACCGCTCGCCGCAGCCGAACGACCGAGCGCAGCGAGTCAGTG |
| | AGCGAGGAAGCGGAAGAGCGCCCAATACGCAAACCGCCTCTCCCCGC |
| | GCGTTGGCCGATTCATTAATGCAGCTGGCACGACAGGTTTCCCGACTG |
| | GAAAGCGGGCAGTGAGCGCAACGCAATTAATGTGAGTTAGCTCACTCA |
| | TTAGGCACCCCAGGCTTTACACTTTATGCTTCCGGCTCGTATGTTGTGT |
| | GGAATTGTGAGCGGATAACAATTTCACACAGGAAACAGCTATGACCAT |
| | GATTACGCCAAGCTTGCATGCCTGCAGGTCGACCTGCAGCGATCGAAC |
| | TTGTTTATTGCAGCTTATAATGGTTACAAATAAAGCAATAGCATCACA |
| | AATTTCACAAATAAAGCATTTTTTTCACTGCATTCTAGTTGTGGTTTGT |
| | CCAAACTCATCAATGTATCTTATCATGTCTGGATCCCTTCTGAGGGCAG |
| | AGGAAGTCTTAATGGCACCGGGCTTGCGGGTCATGCACCAGGTGCGCG |
| | GTCCTTCGGGCACCTCGACGTCGGCGGTGACGGTGAAGCCGAGCCGCT |
| | CGTAGAAGGGGAGGTTGCGGGGCGCGGAGGTCTCCAGGAAGGCGGGC |
| | ACCCCGGCGCGCTCGGCCGCCTCCACTCCGGGGAGCACGACGGCGCTG |
| | CCCAGACCCTTGCCCTGGTGGTCGGGCGAGACGCCGACGGTGGCCAGG |
| | AACCACGCGGGCTCCTTGGGCCGGTGCGGCGCCAGGAGGCCTTCCATC |
| | TGTTGCTGCGCGGCCAGCCGGGAACCGCTCAACTCGGCCATGCGCGGG |
| | CCGATCTCGGCGAACACCGCCCCCGCTTCGACGCTCTCCGGCGTGGTC |
| | CAGACCGCCACCGCGGCGCCGTCGTCCGCGACCCACACCTTGCCGATG |
| | TCGAGCCCGACGCGCGTGAGGAAGAGTTCTTGCAGCTCGGTGACCCGC |
| | TCGATGTGGCGGTCCGGATCGACGGTGTGGCGCGTGGCGGGGTAGTCG |
| | GCGAACGCGGCGGCGAGGGTGCGTACGGCCCTGGGGACGTCGTCGCG |
| | GGTGGCGAGGCGCACCGTGGGCTTGTACTCGGTCATAGCCATGGTGGC |
| | GACGAGGTCGAAAGGCCCGGAGATGAGGAAGAGGAGAACAGCGCGG |
| | CAGACGTGCGCTTTTGAAGCGTGCAGAATGCCGGGCCTCCGGAGGACC |
| | TTCGGGCGCCCGCCCCGCCCCTGAGCCCGCCCCTGAGCCCGCCCCCGG |
| | ACCCACCCTTCCCAGCTGCTGAGCCCAGAAAGCGAAGGAGCAAAGCTG |
| | CTATTGGCCGCTGCCCCAAAGGCCTACCCGCTTCCATTGCTCAGCGGTG |
| | CTGTCCATCTGCACGAGACTAGTGAGACGTGCTACTTCCATTTGTCACG |
| | TCCTGCACGACGCGAGCTGCGGGGCGGGGGGGAACTTCCTGACTAGGG |
| | GAGGAGTAGAAGGTGGCGCGAAGGGGCCACCAAAGAACGGAGCCGGT |
| | TGGCGCTACCGGTGGATGTGGAATGTGTGCGAGGCCAGAGGCCACTTG |
| | TGTAGCGCCAAGTGCCAGCGGGGCTGCTAAAGCGCATGCTCCAGACTG |
| | CCTTGGGAAAAGCGCCTCCCCTACCCGGTAGAATTGGTGGCGATGCCG |
| | AGCTCGGTACCAAGCTTAAGTTTAAACGCTAGCAGTAGAATTCGGATC |
| | CTACCGTTCGTATAGCATACATTATACGAAGTTATGTCGACGATGTAG |
| | GTCACGGTCTCGAAGCCGCGGTGCGGGTGCCAGGGCGTGCCCTTGGGC |
| | TCCCCGGGCGCGTACTCCACCTCACCCATCTGGTCCATCATGATGAACG |
| | GTCGAGGTGGCGGTAGTTGATCCCGGCGAACGCGCGGCGCACCGGGA |
| | AGCCCTCGCCCTCGAAACCGCTGGGCGCGGTGGTCACGGTGAGCACGG |
| | GACGTGCGACGGCGTCGGCGGGTGCGGATACGCGGGGCAGCGTCAGC |
| | GGGTTCTCGACGGTCACGGCGGGCATGTCGACGGCGCGCCGATATCGC |
| | CATGAGTACACCCTGCTGGAATCACGAGGGCTTTCACTGCTGCTGCCTC |
| | GTGGCTGCTCGCCGCCCCGGGTTCAGAAGCGAAAGGCTGGGGCTGCCA |
| | GCACCTTCGGTGGCTTTCGTCAGCACCCGTCAGCCGGGAACATGTGGC |
| | GTGGGGGCACTGCGCGCAGTTTGGAGCTCTCAAGCAGAAGCCACGAAT |
| | CCTTGCCGCTGAAGCGATTCTCTCTGGCCGCATCTGAAACGCCAGGCC |
| | AGAGGAGGTCTGTGCAAGTAACTACTCCCGGAGCTCGCTGACCGCCTC |
| | CCCTCGGGCCGCAGCCGCGGGTCAGAGGGGCAGAGCTCGGGGACTCT |
| | ACGGCTGCCGTGCTGCGCGGGAGGCGGCGGAGCGGCAACGCCGTCCC |
| | GCCCTGCAGTGCTCCGGGCGCTGCCGTCCGGCCTGGGGGCGGCGAGCC |
| | CCTCCCGGCAGTCGCTCGTGACTCCCGCCCGATCCGGCCGCCGCGGGG |
| | CTCTGCTCCTCAGCCCCGCCCCTCCCCGCCGCTCCTCAGCGGAGCGCCG |
| | CGTGCCGCGCTGCCGGGCGGGGCGTTGCGCGGGGGCCGCCGGGAGCG |
| | GTGACGCGTCGCCGAGGCCCCGCCCCGTCGCAGAGTATATAAGGGGCG |
| | GCGCTCACGCCGTCGTTCTTTTCCGCTCCGGGTTTGCCGACGCCGTGCG |
| | GGTGAGGCGCGTGCGGCGGGGCAGGCCGGGCCCGGGGGTGGGGGGTG |
| | CGGGGTGCGGGGATGCTCGGGGGGAGCCTCGTTGCACCGTGAGCGCG |
| | GGGCCGGGGCGCGGCCGCGGAGAGGCGCGTGGGAACGGCGGAGTGGG |
| | GAGGGGGAAGCGAGATGAGGGACGGGTGGGAGAGAGCGGGGCGAGC |
| | GCCGGGGTAAAGCCGACGTGTCCCGCCTGAGGACCCACGCGGGGCAG |
| | CGCGGCGATGAGCACGCGGGGCCCCACCGCCGCCTTGGCTTCGCGCGG |
| | AGCCGCCCGGCGCTGGGGCGCGGCGTACGGTGCTCGGCCTCGGGCGGG |
| | TGGCGGCCGGGGACCCCGGGGCCGCGCCGTCGGTGCCGCGGTGGGGG |
| | AGGGGAGGCGAGGCCGGAGCACGAGAAAAAAAATGGCCGCCCGCTCT |
| | TCCTGCCGCCGCCAGCTCACAAAATGGAGGACGGCGGCCCGGGCGGG |
| | GTGAGTCACACACAAAGGAGAGGGGCGGCCCCGCCCTCCGCGCCGCC |
| | CGTGGCCGCGCCGCCGCCCCGCGCCCCCACCCCAGCCCCTGCCCGGCC |
| | CGGCCGCCCTGCGGGGAGGGGGCGACGGCGGAGGCGCGCTGTAGGGG |
| | ACCCCGGGCCACCGCCCGCCCCTCCGCTCCCCTCCCCCCTCCTCCCCCC |
| | CCTGGCGGGGCGGGGCCGGGGCTGCACGGGCCCTCTGCCCCCTCGGCC |
| | GGCGCCCGGCGGGCGGCGCTGCTGCGGGACGGGGGCGGCCGCAGTAA |
| | CCGGGATTCGTTCTGGAACTTTCAGGTGTCGTTTCTCTTTGGCCGGAAG |
| | AAAGAAGCTAAAGACCATCCGAATCGCGACGCCACCATGTACCCATAC |
| | GATGTTCCAGATTACGCTGACTTACTGGGCCCCATGGAAATGACGGAG |
| | GGCTCCCTCTGCTCCTTCACGGCCGCCGATGACTTCTATGACGACCCGT |
| | GCTTCAACACGTCGGACATGCACTTCTTCGAGGACCTGGACCCCCGGC |
| | TGGTGCACGTGGGCGGGCTGCTGAAAGCCGAGGAGCACCCGCACCAC |
| | CACGGGCACCACCACGGGAACCCACACGAGGAGGAGCACGTGCGGGC |
| | GCCCAGTGGGCACCACCAGGCCGGCCGCTGCCTGCTGTGGGCGTGCAA |
| | GGCCTGCAAGAGGAAGACCACCAACGCTGACCGCCGCAAAGCCGCCA |
| | CCATGAGGGAACGGCGGCGGCTCAGCAAGGTCAACGAGGCCTTTGAG |
| | ACCCTCAAGCGCTGCACTTCCACCAACCCCAACCAGCGCCTGCCCAAG |
| | GTGGAGATCCTGCGCAACGCCATCCGCTACATCGAGAGCCTGCAGGCC |
| | CTGCTGCGTGAGCAGGAGGATGCATACTACCCAGTGCTGGAGCACTAC |
| | AGCGGGGAGTCAGATGCCTCCAGCCCTCGCTCCAACTGCTCCGACGGC |
| | ATGATGGAGTACAGCGGGCCGCCCTGTAGCTCTCGCAGGAGAAACAGC |
| | TACGACAGCAGCTACTACACGGAATCACCAAATGACCCAAAGCATGG |
| | GAAGAGTTCTGTTGTTTCCAGCCTCGACTGCCTCTCAAGCATTGTGGAG |
| | AGGATTTCCACAGACAACTCCACATGTCCCATACTGCCTCCAGCTGAA |
| | GCTGTAGCTGAAGGGAGTCCCTGTTCCCCCCAGGAAGGAGGAAACCTG |
| | AGTGACAGTGGAGCCCAGATTCCTTCCCCCACCAACTGCACCCCTCTTC |
| | CCCAGGAAAGCAGCAGCAGCAGCAGCAGCAATCCAATCTACCAAGTG |
| | CTATAACTCGAGGAGTTGCCAGCCATCTGTTGTTTGCCCCTCCCCCGTG |
| | CCTTCCTTGACCCTGGAAGGTGCCACTCCCACTGTCCTTTCCTAATAAA |
| | ATGAGGAAATTGCATCATAACTTCGTATAGCATACATTATACGAACGG |
| | TAGGATCCCCGGGTACCGAGCTCGAATTCACTGGCCGTCGTTTTACAA |
| | CGTCGTGACTGGGAAAACCCTGGCGTTACCCAACTTAATCGCCTTGCA |
| | GCACATCCCCCTTTCGCCAGCTGGCGTAATAGCGAAGAGGCCCGCACC |
| | GATCGCCCTTCCCAACAGTTGCGCAGCCTGAATGGCGAATGGCGCCTG |
| | ATGCGGTATTTTCTCCTTACGCATCTGTGCGGTATTTCACACCGCATAT |
| | GGTGCACTCTCAGTACAATCTGCTCTGATGCCGCATAGTTAAGCCAGC |
| | CCCGACACCCGCCAACACCCGCTGACGCGCCCTGACGGGCTTGTCTGC |
| | TCCCGGCATCCGCTTACAGACAAGCTGTGACCGTCTCCGGGAGCTGCA |
| | TGTGTCAGAGGTTTTCACCGTCATCACCGAAACGCGCGAGACGAAAGG |
| | GCCTCGTGATACGCCTATTTTTATAGGTTAATGTCATGATAATAATGGT |
| | TTCTTAGACGTCAGGTGGCACTTTTCGGGGAAATGTGCGCGGAACCCC |
| | TATTTGTTTATTTTTCTAAATACATTCAAATATGTATCCGCTCATGAGA |
| | CAATAACCCTGATAAATGCTTCAATAATATTGAAAAAGGAAGAGT |

What is claimed is:

1. A method for improving myogenic differentiation capacity of a cell line, the method comprising:
(a) contacting the cell line with a culture media comprising:
(i) at least a first Activin A inhibitor,
(ii) at least a first BMP inhibitor, or
(iii) at least a first WNT activator, or a combination thereof,
(b) inducing myogenic specific differentiation, comprising inducing formation of myocytes, myoblasts, myotubes, or a combination thereof,
thereby improving the cell line's myogenic differentiation capacity as compared to a control.

2. The method of claim 1, further comprising, prior to step (a), isolating a population of cells from skin or muscle tissue to form a cell line.

3. The method of claim 2, further comprising immortalizing the cell line, wherein the immortalizing step comprises introducing into, or incorporating into the genome of, a cell of the cell line a polynucleotide encoding a telomerase reverse transcriptase (TERT) polypeptide, thereby generating an immortalized cell line.

4. The method of claim 1, wherein the cell line is a late passage cell line.

5. The method of claim 4, wherein the late passage cell line has lost myogenic differentiation capacity.

6. The method of claim 1, further comprising introducing into, or incorporating into the genome of, a cell of the cell line a polynucleotide encoding at least a first myogenic regulatory factor polypeptide, wherein the at least first myogenic regulatory factor is selected from: MYOD, MYOG, MEF2B, PAX7, PAX3, and PITX1.

7. The method of claim 6, wherein the polynucleotide comprising the first myogenic regulatory factor polypeptide further comprises a nucleic acid sequence encoding a second myogenic regulatory factor polypeptide, a nucleic acid sequence encoding a third myogenic regulatory factor polypeptide, or a combination thereof,
wherein the first myogenic regulatory factor polypeptide is a PAX7 polypeptide or a fragment thereof, the second myogenic regulatory factor polypeptide is a MEF2B polypeptide or a fragment thereof, and the third myogenic regulatory factor polypeptide is a MYOD polypeptide or a fragment thereof).

8. The method of claim 1, wherein the Activin A inhibitor is selected from: A-83-01, E-616542, SB431542, TGFβRI-IN-3, R-268712, Follistatin, and Follistatin-like-3.

9. The method of claim 1, wherein the BMP inhibitor is selected from: LDN193189, Noggin, Chrodin, and Gremlin.

10. The method of claim 1, wherein the WNT activator is selected from: CHIR99021, BIO, AZD1080, WNT1a, WNT3a, WNT4, and WNT7.

11. The method of claim 1, further comprising contacting the cell line with a culture media comprising a histone deacetylase inhibitor.

12. The method of claim 1, wherein the cell line is derived from a species selected from: poultry, livestock, game, or aquatic animal species.

13. The method of claim 1, wherein the cell line is a fibroblast cell line.

14. The method of claim 1, wherein the cell line is not a pluripotent stem cell line.

15. The method of claim 1, wherein inducing myogenic-specific differentiation comprises contacting the cell line with a differentiation medium.

16. A method for restoring myogenic differentiation capacity of a late passage cell line, the method comprising:
(a) contacting the late passage cell line with a culture media comprising:
(i) at least a first Activin A inhibitor,
(ii) at least a first BMP inhibitor, or
(iii) at least a first WNT activator, or a combination thereof; and
(b) inducing myogenic specific differentiation, comprising inducing formation of myocytes, myoblasts, myotubes, or a combination thereof,
thereby restoring the late passage cell line's myogenic differentiation capacity as compared to a late passage control cell line.

17. The method of claim 16, wherein the late passage cell line has exceeded 60 population doublings.

18. The method of claim 17, wherein the late passage control cell line has lost myogenic differentiation capacity at or above 60 population doublings.

19. A population of myocytes, myoblasts, myotubes, multinucleated myotubes, satellite cells, skeletal muscle fibers, or any combination thereof produced by the method of claim 1.

20. An in vitro method for producing a cell-based meat product, the method comprising:
forming the myocytes, myoblasts, myotubes, or a combination thereof, of claim 1 into a cell based meat product.
